SEQUENCE LISTING (12 sequences)

SEQ ID NO: 1 - protein, 107 aa, Artificial Sequence
  Synthetic construct - humanized antibody sequence
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIK

SEQ ID NO: 2 - protein, 119 aa, Artificial Sequence
  Synthetic construct - humanized antibody sequence
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 3 - DNA, 357 bp, Artificial Sequence
  Synthetic construct - polynucleotide encoding humanized antibody sequence
gaagtgcagctggtggagtctgggggaggcttagtgaagcctggagggtccctgaaactctcctgtgcagcctctggattcaagtttagtagatatgccatgtcttgggttcgccaggctccggggaagaggctggagtgggtcgcaaccattagtagtggtggtagttacatctactatccagacagtgtgaagggtcgattcaccatctccagagacaatgtcaagaacaccctgtatctgcaaatgagcagtctgaggtctgaggacacggccatgtattactgtgcaagacgagattacgacctggactactttgactcctggggccaaggcacccttgtcaccgtctcctca

SEQ ID NO: 4 - DNA, 321 bp, Artificial Sequence
  Synthetic construct - polynucleotide encoding humanized antibody sequence
gacatccagatgacccagtctccatcctccctgtctgcatcggtgggagacagagtcactatcacttgcaaggcgagtcgggacattagaagctatttaacctggtaccagcagaaaccagggaaagctcctaagaccctgatctattatgcaacaagcttggcagatggggtcccgtcgagattcagtggcagtggatctgggcaagattattctctcaccatcagcagcctggagtctgacgatacagcaacttactactgtctacaacatggtgagagtccattcacgctcggctcggggaccaagctggaaatcaaa

SEQ ID NO: 5 - protein, 449 aa, Artificial Sequence
  Synthetic construct - humanized antibody sequence
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Synthetic construct - humanized antibody sequence
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 10 aa, Artificial Sequence
  Synthetic construct - humanized antibody sequence
GFKFSRYAMS

SEQ ID NO: 8 - protein, 17 aa, Artificial Sequence
  Synthetic construct - humanized antibody sequence
TISSGGSYIYYPDSVKG

SEQ ID NO: 9 - protein, 10 aa, Artificial Sequence
  Synthetic construct - humanized antibody sequence
RDYDLDYFDS

SEQ ID NO: 10 - protein, 11 aa, Artificial Sequence
  Synthetic construct - humanized antibody sequence
KASRDIRSYLT

SEQ ID NO: 11 - protein, 7 aa, Artificial Sequence
  Synthetic construct - humanized antibody sequence
YATSLAD

SEQ ID NO: 12 - protein, 7 aa, Artificial Sequence
  Synthetic construct - humanized antibody sequence
LQHGESP

(19) United States
(12) Patent Application Publication
(10) Pub. No.: US 2024/0053360 A1
(43) Pub. Date: Feb. 15, 2024

(54) METHODS OF SELECTIVELY TARGETING CD6 HIGH CELLS AND DECREASING ACTIVITY OF TEFF CELLS

(71) Applicant: Equillium, Inc., La Jolla, CA (US)

(72) Inventors: Stephen CONNELLY, Rancho Santa Fe, CA (US); Jeanette AMPUDIA, San Diego, CA (US); Nhu (Dalena) Ngo CHU, Santee, CA (US); Cherie T. NG, San Diego, CA (US)

(21) Appl. No.: 18/265,121

(22) PCT Filed: Dec. 3, 2021
(86) PCT No.: PCT/US2021/061904

§ 371 Date: Jun. 2, 2023

Related U.S. Application Data
(60) Provisional application No. 63/121,567, filed on Dec. 4, 2020.

(51) Int. Cl. G01N 33/68; G01N 33/569; G01N 33/564

(52) U.S. Cl. CPC G01N 33/6872; G01N 33/56972; G01N 33/564; G01N 2333/70596; G01N 2500/04; G01N 2500/10; G01N 2800/245

(57) ABSTRACT

Provided are methods and compositions comprising anti-CD6 antibodies, such as itolizumab, which selectively target CD6^{high} T cells, for example, to reduce levels of cell surface CD6 on such cells, decrease the overall levels and pathogenic activity of CD6^{high} T cells or the ratio of CD6^{high}:CD6^{low} T cells in a subject or ex vivo, decrease the overall levels and pathogenic activity of T_{eff} cells or the ratio of T_{eff}:T_{reg} cells in a subject or ex vivo, and/or increase generation of T_{reg} cells in a subject or ex vivo, and thereby modulate a pathogenic immune response in the subject, among other aspects.

### CROSS-REFERENCE TO RELATED APPLICATIONS

This application claims the benefit under 35 U.S.C. § 119(e) of U.S. Provisional Application No. 63/121,567, filed Dec. 4, 2020, which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is EQIL_010_01WO_ST25.txt. The text file is about 10.8 KB, was created on Dec. 2, 2021, and is being submitted electronically via EFS-Web.

### BACKGROUND

### Technical Field

Embodiments of the present disclosure relate generally to methods and compositions comprising anti-CD6 antibodies, such as itolizumab, which selectively target CD6^{high} T cells, for example, to reduce levels of cell surface CD6 on such cells, decrease the overall levels and pathogenic activity of CD6^{high} T cells or the ratio of CD6^{high}:CD6^{low} T cells in a subject or ex vivo, decrease the overall levels and pathogenic activity of T_{eff} cells or the ratio of T_{eff}:T_{reg} cells in a subject or ex vivo, and/or increase generation of T_{reg} cells in a subject or ex vivo, and thereby modulate a pathogenic immune response in a subject, among other aspects.

### Description of the Related Art

T cells or T-lymphocytes belong to a group of white blood cells known as lymphocytes, and play a central role in adaptive immunity. They can be distinguished from other lymphocyte types, such as B cells, by the presence of a special receptor on their cell surface called T cell receptors (TCR). There are two main groups—gamma delta T cells (γδT cells) and alpha beta T cells (αβ T cells); the latter includes CD4 and CD8 T cells. CD4 T cells include the T helper (Th) subsets such as Th1, Th2, Th9, Th17, Th22, Tfh, and Tph which each have distinct cytokine profiles and roles in immune responses. CD8 T cells include cytotoxic T cells (Tc cells or CTLs). T cells are also defined by activation status which includes naïve, effector and memory (central, effector and stem memory) subsets. Other related cells include natural killer T cells (NKT cells) and innate lymphoid cells (ILCs). ILCs are TCR negative lymphocytes but have subsets, ILC1s, ILC2s and ILC3s, that are analogous to different Th subsets in terms of the cytokines they secrete. All T cells (or ILCs) which have been activated (no longer naïve) are collectively referred to as effector T cells (T_{eff}).

Regulatory T cells (T_{reg} cells), also known as suppressor T cells, are a specialized subpopulation of T cells that act to suppress immune responses of other T cells. For example, T_{reg} cells play a major role in suppressing T cell-mediated immunity during an immune reaction and in suppressing auto-reactive T cells that escaped the process of negative selection in the thymus. As such, T_{reg} cells provide an important “self-check” to prevent excessive immunogenic reactions and are thus crucial for the maintenance of immune system homeostasis and tolerance to self-antigens. An increase in number and activity of T_{eff} cells leading to an increased ratio of T_{eff}:T_{reg} cells underscores autoimmune and inflammatory disease.

Two major classes of T_{reg} cells are the naturally-occurring T_{reg} cells and the induced T_{reg} cells. The most widely used markers for naturally-occurring T_{reg} cells are cluster of differentiation 4 (CD4), cluster of differentiation 25 (CD25), forkhead/winged-helix transcription factor box P3 (FoxP3), transcription factor Helios, cytotoxic T-lymphocyte-associated antigen 4 (CTLA-4), glucocorticoid-induced tumor necrosis factor receptor family-related gene (GITR), lymphocyte activation gene-3 (LAG-3), and cluster of differentiation 127 (CD127). Naturally occurring T_{reg} cells (also known as CD4+CD25+FoxP3+T_{reg} cells) arise in the thymus. Induced T_{reg} cells (including Tr1 cells, Th3 cells, and HLA-G+T_{reg} cells) originate in the periphery during a normal immune response in response to interactions with antigen presenting cells and cytokines.

Induced T_{reg} cells share many of the attributes of naturally-occurring T_{reg} cells but can differ in critical cell surface and nuclear biomarkers and functional attributes. For instance, Tr1 and Th3 cells have been described that produce IL-10 and TGF-β, respectively. The ability to isolate, enrich, and expand these cell subsets has led to novel therapeutic approaches in treating immunological diseases.

The identification of naturally-occurring T_{reg} cells as an important component of self-tolerance has opened a major area of investigation in immunology and the basic processes that control immune tolerance. Regulatory T cells have a unique and robust therapeutic profile. The cells require specific T cell receptor (TCR)-mediated activation to develop regulatory activity, but their effector function appears to be nonspecific, regulating local inflammatory responses through a combination of cell-cell contact and suppressive cytokine production. Numerous studies have demonstrated the potent influence of naturally-occurring T_{reg} cell in suppressing pathologic immune responses in autoimmune diseases, transplantation, and graft-vs-host diseases. However, a major obstacle to the study and application of naturally-occurring T_{reg} cells in the human setting has been the lack of a single specific cell surface biomarker to define and separate T_{reg} cells from other subsets of T cells such as, e.g., Th cells, Tc cells, as well as to distinguish between different subpopulations of T_{reg} cells.

A number of different methods are employed in research to identify, isolate, enrich, or otherwise exploit T_{reg} cells. The markers CD4, CD25, FoxP3, transcription factor Helios, CTLA-4, GITR, LAG-3, and CD127 are used in concert to identify T_{reg} cells, however, none of the above-listed markers can be used alone to identify T_{reg} cells as none are strictly T_{reg} cell-specific. For example, high expression of CD25 and CD4 surface markers (CD4+CD25+ cells) was originally used to identify naturally-occurring T_{reg} cells. However, CD4 is also expressed on Th cells, and a subpopulation of TM cells. CD25 is also expressed on non-regulatory T cells in the setting of immune activation such as during an immune response to a pathogen. Thus, as defined by CD4 and CD25 expression, T_{reg} cells comprise about 5-10% of mature Th cells. The additional measurement of cellular expression of Foxp3 allowed a more specific analysis of naturally-occurring T_{reg} cells (CD4+CD25+FoxP3+ cells). However, Foxp3 is also transiently expressed in activated TEM cells and it is now well documented that most human CD4+ and CD8+ T cells transiently express Foxp3 upon activation, including CD4+CD25 low/˜T cells, Th cells, Tc cells, and memory T cells. Furthermore, FoxP3 is a nuclear marker that requires cell membrane permeabilization prior to staining. As such, use of this biomarker precludes subsequent processing steps such as separating, isolating, enriching, or expanding viable cells.

There exists a need for improved methods of selectively targeting subsets of T cells and methods of selectively inhibiting the population of T_{eff} cells as well as methods of modulating an immune reaction in an individual. Additionally, there is a need to develop compositions comprising a population of immunosuppressive regulatory T-cells for modulating an immune reaction in an individual.

### BRIEF SUMMARY

Embodiments of the present disclosure include methods for determining an optimal dosage of itolizumab in a human subject having an autoimmune, immuno-inflammatory, or inflammatory disease, graft versus host disease (GVHD), or organ transplant rejection, comprising:
(a) determining a baseline of cell surface CD6 levels in a tissue sample from the subject, wherein the tissue sample comprises T-lymphocytes, and optionally defining a target level of cell surface CD6 in the subject;
(b) administering to the subject a series of two or three or more dosages of itolizumab, optionally increasing dosages;
(c) monitoring cell surface CD6 levels in a tissue sample from the subject between the series of dosages, wherein the tissue sample comprises T-lymphocytes;
(d) identifying the lowest dosage from the series of dosages as being the optimal dosage if cell surface CD6 levels in step (c) are about or less than about 5, 10, 15, 20, 25, 30, 40, 45, or 50 percent of the baseline from (a), or are within about or less than about 5, 10, 15, or 20 percent of the optional target level from (a), and if no further reductions in cell surface CD6 levels are observed between the series of dosages.

Certain embodiments comprise determining cell surface CD6 levels on CD4 cells and/or CD8 cells in the tissue sample from the subject. In some embodiments, the tissue sample is a blood sample. Particular embodiments comprise defining the target level of cell surface CD6 in the subject based on clinical parameters or symptoms of the disease.

Also included are dosing regimens for treatment of an autoimmune, immuno-inflammatory, or inflammatory disease, graft versus host disease (GVHD), or organ transplant rejection in a human subject in need thereof, comprising:
(a) determining a baseline of cell surface CD6 levels in a tissue sample from the subject, wherein the tissue sample comprises T-lymphocytes, and optionally defining a target level of cell surface CD6 in the subject;
(b) administering to the subject a dosage of itolizumab, which reduces cell surface CD6 levels in T-lymphocytes in the subject to about or less than about 5, 10, 15, 20, 25 percent of the baseline from (a);
(c) monitoring cell surface CD6 levels in a tissue sample from the subject, wherein the tissue sample comprises T-lymphocytes; and
(d) administering a further dosage of itolizumab to the subject before or if the cell surface CD6 levels in (c) return to about or more than about 25, 30, 35, 40, 45, 50, 55, 60, 65, 70, or 75 percent of the baseline from (a), or rise to about or above the target level from (a).

In some embodiments, the dosing regimen maintains cell surface CD6 levels in T-lymphocytes (optionally CD4 and/or CD8 cells) from the subject at about or lower than about 40, 45, 50, 55, 60, 65, 70, or 75 percent of the baseline from (a), or within about 5, 10, 15, or 20 percent of the optional target level from (a), optionally defining the target level of cell surface CD6 in the subject based on clinical parameters or symptoms of the disease.

Some embodiments relate to dosing regimens for treatment of an autoimmune, immuno-inflammatory, or inflammatory disease, graft versus host disease (GVHD), or organ transplant rejection in a human subject in need thereof, comprising:
(a) determining a baseline level of CD6^{high} T-lymphocytes in a blood sample from the subject, and optionally defining a target level of CD6^{high} T-lymphocytes;
(b) administering to the subject a dosage of itolizumab, which reduces the level of CD6^{high} T-lymphocytes in the subject to about or less than about 5, 10, 15, 20, 25, 30, or 40 percent of the baseline from (a);
(c) monitoring levels of CD6^{high} T-lymphocytes in a blood sample from the subject; and
(d) administering a further dosage of itolizumab to the subject before the levels of CD6^{high} T-lymphocytes from (c) return to about or more than about 25, 30, 35, 40, 45, 50, 55, 60, 65, 70, or 75 percent of the baseline level from (a), or rise to about or above the target level from (a), optionally defining the target level of CD6^{high} T-lymphocytes in the subject based on clinical parameters or symptoms of the disease.

In some embodiments, the dosing regimen maintains levels of CD6^{high} T-lymphocytes in the subject at about or less than about 45, 50, 55, 60, 65, 70, or 75 percent of the baseline level from (a), optionally wherein the T-lymphocytes are CD4 cells and/or CD8 cells. In some embodiments, the dosing regimen decreases the ratio of CD6^{high}:CD6^{low} T-lymphocytes in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally wherein the T-lymphocytes are CD4 cells and/or CD8 cells. In some embodiments, the dosing regimen decreases the ratio of T_{eff}:T_{reg} cells in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally CD4 cells and/or CD8 cells, optionally wherein the T_{eff} cells are Th17 cells.

Certain embodiments include methods of preventing or ameliorating graft versus host disease (GVHD) in a human transplant patient comprising:
(a) incubating a transplant tissue with an anti-CD6 antibody, optionally itolizumab, for a time sufficient to reduce cell surface levels of CD6 on CD6^{high} T-lymphocytes in the transplant tissue; and
(b) transplanting the transplant tissue into the patient.

Some embodiments comprise determining cell surface levels of CD6 on T-lymphocytes in the transplant tissue before and after step (a), and performing step (b) if cell surface levels of CD6 after step (a) are reduced by about or at least about 10, 20, 30, 40, 50, 60, 70, 80, 90, 100, 200, 300, 400, 500 percent or more relative to before step (a).

In some embodiments, the transplant tissue comprises umbilical cord blood cells, bone marrow cells, peripheral blood cells, mobilized peripheral blood cells, mesenchymal stem cells, hematopoietic stem cells, cells differentiated from stem cells/progenitor cells, engineered cells (optionally chimeric antigen receptor (CAR) cells), or any combinations of said cells. In some embodiments, the transplant tissue is autologous to the human transplant patient. In some embodiments, the transplant tissue is allogeneic to the human transplant patient.

Also included are methods for treatment or amelioration of an autoimmune, immuno-inflammatory, or inflammatory disease in a human patient in need thereof, comprising:
(a) incubating a transplant tissue with an anti-CD6 antibody, optionally itolizumab, for a time sufficient to reduce cell surface levels of CD6 on CD6^{high} T-lymphocytes in the transplant tissue, thereby generating transplant tissue enriched for CD6^{low} T-lymphocytes;
(b) treating the transplant tissue from (a) for a time sufficient to generate T_{reg} lymphocytes from the CD6^{low} T-lymphocytes; and
(c) transplanting the transplant tissue from (c) into the patient.

Certain embodiments comprise determining cell surface levels of CD6 on T-lymphocytes in the transplant tissue before and after step (a), and performing step (b) if cell surface levels of CD6 after step (a) are reduced by about or at least about 10, 20, 30, 40, 50, 60, 70, 80, 90, 100, 200, 300, 400, 500 percent or more relative to before step (a). In some embodiments, step (b) comprises incubating the transplant tissue enriched for CD6^{low} T-lymphocytes with a combination of cytokines, growth factors, and transcription factors for a time sufficient to generate T_{reg} lymphocytes.

In some embodiments, the transplant tissue comprises umbilical cord blood cells, bone marrow cells, peripheral blood cells, mobilized peripheral blood cells, mesenchymal stem cells, hematopoietic stem cells, cells differentiated from stem cells/progenitor cells, engineered cells (optionally chimeric antigen receptor (CAR) cells), or any combinations of said cells. In some embodiments, the transplant tissue is autologous to the human patient. In some embodiments, the transplant tissue is allogeneic to the human patient.

Also included are methods for treatment of an autoimmune, immuno-inflammatory, or inflammatory disease, graft versus host disease (GVHD), or organ transplant rejection in a human subject in need thereof, comprising:
(a) administering itolizumab to the subject; and
(b) determining levels of cell surface CD6 on T-lymphocytes in a tissue sample from the subject, determining levels of CD6^{high} and optionally CD6^{low} T-lymphocytes in a tissue sample from the subject, and/or determining levels of T_{eff} and T_{reg} cells in the subject, wherein the tissue sample comprises T-lymphocytes;
wherein the administration of itolizumab reduces any one or more of (i) levels of cell surface CD6 on T-lymphocytes, optionally CD4 and/or CD8 cells; (ii) levels of CD6^{high} T-lymphocytes in the subject; (iii) the ratio of CD6^{high}:CD6^{low} T-lymphocytes in the subject; and/or (iv) the ratio of T_{eff}:T_{reg} cells in the subject, and thereby reduces a pathogenic immune response in the subject.

In some embodiments, the administration of itolizumab:
decreases cell surface CD6 levels on T-lymphocytes in the subject by about or at least about 10, 20, 30, 40, 50, 60, 70, 80, 90, 100, 200, 300, 400, 500 percent or more relative to a control or standard or baseline, optionally wherein the T-lymphocytes are CD4 cells and/or CD8 cells;
decreases levels of CD6^{high} T-lymphocytes in the subject by about or at least about 10, 20, 30, 40, 50, 60, 70, 80, 90, 100, 200, 300, 400, 500 percent or more relative to a control or standard or baseline, optionally wherein the T-lymphocytes are CD4 cells and/or CD8 cells;
decreases the ratio of CD6^{high}:CD6^{low} T-lymphocytes in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally wherein the T-lymphocytes are CD4 cells and/or CD8 cells; and/or
decreases the ratio of T_{eff}:T_{reg} cells in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally wherein the T_{eff} cells are Th17 cells.

Particular embodiments include in vitro cell-based (i.e., cell culture-based) method for analyzing a test lot of itolizumab, comprising
(a) incubating the test lot of itolizumab with cells that express CD6 on the cell surface; (b) measuring cell surface CD6 expression on the cells; and
(c) formulating the test lot of itolizumab as a pharmaceutical composition if the test lot decreases cell surface CD6 expression relative to a control or standard, and rejecting the test lot of itolizumab if the test lot does not decrease cell surface CD6 expression relative to the control or standard.

In some embodiments, step (b) comprises directly measuring cell surface CD6 expression by flow cytometry, cytometry by time-of-flight (CyToF), cellular ELISA, or immunofluorescent microscopy, or wherein (b) comprises measuring soluble CD6 in supernatant as an indicator of cell surface CD6 expression, optionally by enzyme-linked immunosorbent assay (ELISA), chemiluminescence assay, electrochemiluminescence assay, high performance liquid chromatography, western blot, or immunoprecipitation followed by western blot. In some embodiments, the cells comprise peripheral blood mononuclear cell (PBMCs). In some embodiments, the cells comprise a human T cell line or a cell line engineered to express CD6, optionally human CD6. In some embodiments, the cell line is selected from MOLT-4, MOLT-3, MOLT-16, HuT 78, HuT 102, Jurkat, Jurkat NFAT, CCRF-CEM, 12.1, MJ (G11), LOUCY, SUP-T1, HEL.92.1.7, EFO-21, RPMI-8226, HPB-ALL, HH, KE37, P12ICHIKAWA, PEER, ALLSIL, RPMI8402, CMLT1, PF382, EHEB, and DU4475 cells. In some embodiments, the cells comprise monocytes, optionally a monocyte cell line. In some embodiments, the monocyte cell line is selected from U937, THP1, MC-1010, TUR, AML-193, and MV-4-11. In some embodiments, (a) the human T cell line or cell line engineered to express CD6 and (b) the monocytes, optionally a monocyte cell line, are present at a ratio of about 30:1, 25:1, 20:1, 15:1, 10:1, 5:1, 4:1, 3:1, 2:1, 1:1, 1:2, 1:3, 1:4, 1:5, or 1:10.

Certain embodiments comprise formulating the test lot of itolizumab as a pharmaceutical composition if it decreases cell surface CD6 expression by about or at least about 10, 20, 30, 40, 50, 60, 70, 80, 90, 100, 200, 300, 400, 500 percent or more relative to the control or standard and/or if it increases soluble CD6 in supernatant by about or at least about 10, 20, 30, 40, 50, 60, 70, 80, 90, 100, 200, 300, 400, 500 percent or more relative to the control or standard.

Also included are methods of screening an anti-CD6 antibody, or antigen binding fragment thereof, for use as a biological therapeutic comprising:
(a) incubating the candidate anti-CD6 antibody, or antigen binding fragment thereof, with cells that express CD6 on the cell surface;
(b) measuring cell surface CD6 expression on the cells; and
(c) formulating the candidate anti-CD6 antibody, or antigen binding fragment thereof, as a pharmaceutical composition if it decreases cell surface CD6 expression relative to a control or standard.

In some embodiments, step (b) comprises directly measuring cell surface CD6 expression by flow cytometry, cytometry by time-of-flight (CyToF), cellular ELISA, or immunofluorescent microscopy, or wherein (b) comprises measuring soluble CD6 in supernatant as an indicator of cell surface CD6 expression, optionally by enzyme-linked immunosorbent assay (ELISA), chemiluminescence assay, electrochemiluminescence assay, high performance liquid chromatography, western blot, and immunoprecipitation followed by western blot. In some embodiments, the cells comprise peripheral blood mononuclear cell (PBMCs). In some embodiments, the cells comprise a human T cell line or a cell line engineered to express CD6, optionally human CD6. In some embodiments, the cell line is selected from MOLT-4, MOLT-3, MOLT-16, HuT 78, HuT 102, Jurkat, Jurkat NFAT, CCRF-CEM, 12.1, MJ (G11), LOUCY, SUP-T1, HEL.92.1.7, EFO-21, RPMI-8226, HPB-ALL, HH, KE37, P12ICHIKAWA, PEER, ALLSIL, RPMI8402, CMLT1, PF382, EHEB, and DU4475 cells. In some embodiments, the cells comprise monocytes, optionally a monocyte cell line. In some embodiments, the monocyte cell line is selected from U937, THP1, MC-1010, TUR, AML-193, and MV-4-11 In some embodiments, (a) the human T cell line or cell line engineered to express CD6 and (b) the monocytes, optionally a monocyte cell line, are present at a ratio of about 30:1, 25:1, 20:1, 15:1, 10:1, 5:1, 4:1, 3:1, 2:1, 1:1, 1:2, 1:3, 1:4, 1:5, or 1:10.

Certain embodiments comprise formulating the candidate anti-CD6 antibody, or antigen binding fragment thereof, as a pharmaceutical composition if it decreases cell surface CD6 expression by about or at least about 10, 20, 30, 40, 50, 60, 70, 80, 90, 100, 200, 300, 400, 500 percent or more relative to a control or standard and/or if it increases soluble CD6 in supernatant by about or at least about 10, 20, 30, 40, 50, 60, 70, 80, 90, 100, 200, 300, 400, 500 percent or more relative to the control or standard.

In certain of the methods or dosing regimens, the subject or patient with the autoimmune, immuno-inflammatory, or inflammatory disease has an increased ratio of T_{eff}:T_{reg} cells relative to a standard or healthy subject, including wherein the T_{eff} cells are Th17 cells. In some embodiments, the ratio of T_{eff}:T_{reg} cells is increased by about or at least about 1.5, 2, 3, 4, 5, 6, 7, 8, 9, or 10-fold or more relative to the standard or healthy subject. In some embodiments, the autoimmune, immuno-inflammatory, or inflammatory disease is inflammatory bowel disease (IBD), optionally Crohn's disease or ulcerative colitis, systemic lupus erythematosus (SLE), optionally SLE with lupus nephritis, rheumatoid arthritis (RA), multiple sclerosis (MS), psoriasis, psoriatic arthritis, ankyolosing spondylitis, or asthma.

### BRIEF DESCRIPTION OF THE DRAWINGS

Itolizumab causes loss of cell surface CD6 expression. FIG. 1 shows the level of surface expression of CD6 in CD4+CD45RA− cells (effector and memory CD4 T cells) following a single treatment with itolizumab as compared to isotype control and the loss of cell surface CD6 starting at ten minutes and over the course of 24 hours as measured by flow cytometry.

Itolizumab causes dose-dependent loss of CD6 expression on CD4 and CD8 T cells in vitro. FIGS. 2A-2D are graphs of the mean fluorescent intensities of cell surface CD6 after treatment with four different concentrations of itolizumab; 0.01, 0.1, 1, 10, and 100 ug/mL. FIG. 2A are CD4+CD45RA+ cells (naïve T cells); FIG. 2B are CD4+CD45RA− cells; FIG. 2C are CD8+CD45RA+ cells (naïve CD8 T cells); and FIG. 2D are CD8+CD45RA− cells (effector and memory CD8 T cells). In each the mean fluorescent intensity of cell surface CD6 is normalized to isotype (10 ug/mL) treated cells. FIG. 2E shows that cell surface loss of CD6 (left graph) correlates with increase in soluble CD6 (sCD6) in the supernatant (right graph) by an electrochemiluminescent assay. Here, PBMCs from 3 different donors were incubated with 10 ug/mL of itolizumab and cell surface expression of CD6 (left graph) was assessed on CD4 T cells and soluble CD6 (right graph) was quantified in the PBMC supernatant at the indicated timepoints. The calculated number of CD6 receptors on CD4 T cells at baseline as assessed by flow cytometry was used to normalize values across the 3 donors. *** p<0.001, ** p<0.01, * p<0.05.

Patients treated with itolizumab have decreased CD6 expression on CD4 and CD8 T cells. FIGS. 3A-3J show graphs of the mean fluorescent intensities of cell surface CD6 as a percentage of baseline on the Y axis and days after initial treatment on the X axis, measured in all CD4 positive cells and all CD8 positive cells isolated from GVHD patients treated with itolizumab. FIGS. 3A-3D are four patients treated with from 1 to 5 doses of 0.4 mg/kg itolizumab administered biweekly. FIG. 3A is a graph of the percentage of cell surface CD6 in CD4 and CD8 positive cells as compared to baseline for a patient treated with 0.4 mg/kg itolizumab on days 1, 15, 29, 43, and 57. FIG. 3B is a graph of the percentage of cell surface CD6 in CD4 and CD8 positive cells as compared to baseline for a patient treated with 0.4 mg/kg itolizumab on days 1, 15, and 29. FIG. 3C is a graph of the percentage of cell surface CD6 in CD4 and CD8 positive cells as compared to baseline for a patient treated with 0.4 mg/kg itolizumab on days 1, 15, 29, 43, and 57. FIG. 3D is a graph of the percentage of cell surface CD6 in CD4 and CD8 positive cells as compared to baseline for a patient treated with 0.4 mg/kg itolizumab on day 1. FIGS. 3E-3G are three patients treated with from 2 to 4 doses of 0.8 mg/kg itolizumab administered biweekly or intermittent biweekly. FIG. 3E is a graph of the percentage of cell surface CD6 in CD4 and CD8 positive cells as compared to baseline for a patient treated with 0.8 mg/kg itolizumab on days 1, 15, and 29. FIG. 3F is a graph of the percentage of cell surface CD6 in CD4 and CD8 positive cells as compared to baseline for a patient treated with 0.8 mg/kg itolizumab on days 1, 15, 43, and 57. FIG. 3G is a graph of the percentage of cell surface CD6 in CD4 and CD8 positive cells as compared to baseline for a patient treated with 0.8 mg/kg itolizumab on days 1 and 15. FIGS. 3H-3J are three patients treated with from 2 to 5 doses of 1.6 mg/kg itolizumab administered biweekly. FIG. 3H is a graph of the percentage of cell surface CD6 in CD4 and CD8 positive cells as compared to baseline for a patient treated with 1.6 mg/kg itolizumab on days 1, 15, 29, 43 and 57. FIG. 3I is a graph of the percentage of cell surface CD6 in CD4 and CD8 positive cells as compared to baseline for a patient treated with 1.6 mg/kg itolizumab on days 1 and 15. FIG. 3J is a graph of the percentage of cell surface CD6 in CD4 and CD8 positive cells as compared to baseline for a patient treated with 1.6 mg/kg itolizumab on days 1, 15, 29, and 43.

Dose responsive loss of CD6 in itolizumab treated patients. FIGS. 4A-4D are bar graphs of the mean fluorescent intensity of cell surface CD6 for patients treated with 0.4 mg/kg, 0.8 mg/kg, and 1.6 mg/kg itolizumab (left to right in the portion of each graph). FIG. 4A is a bar graph of CD6 surface expression as compared to baseline in CD4 positive cells and CD8 positive cells 24 hours after 1 dose of 0.4 mg/kg, 0.8 mg/kg, or 1.6 mg/kg itolizumab. FIG. 4B is a bar graph of the percent intensity of surface CD6 expression compared to baseline in CD4 positive cells and CD8 positive cells 8 days following 1 dose of 0.4 mg/kg, 0.8 mg/kg, or 1.6 mg/kg itolizumab. FIG. 4C is a bar graph of the percent intensity of surface CD6 expression compared to baseline in CD4 positive cells and CD8 positive cells on day 15 following 1 dose of 0.4 mg/kg, 0.8 mg/kg, or 1.6 mg/kg itolizumab. FIG. 4D is a bar graph of the percent intensity of surface CD6 expression compared to baseline in CD4 positive cells and CD8 positive cells on day 29 following 2 biweekly dose of 0.4 mg/kg, 0.8 mg/kg, or 1.6 mg/kg itolizumab.

Binding of itolizumab decreases faster than the rate of CD6 loss. FIGS. 5A-5F are plotted data points of the percentage of positive events at each timepoint showing CD6 labeling, both unbound receptor and receptor bound to a non-competitive anti-CD6 antibody clone, on two different cell types at two different concentrations of itolizumab. Unbound receptor (squares) is detected at each timepoint by staining cells with a fluorescently labeled itolizumab. Fluorescently labeled itolizumab will bind to all available CD6 not already bound by the unlabeled itolizumab where CD6 is still present on the surface of cells. Binding of a proprietary non-competitive antibody (circles) is used to detect levels of total CD6 on the surface of cells at each timepoint. When cells are treated with itolizumab, there is a decrease in total levels of surface CD6 which is also reflected in a decrease in the amount of unbound receptor (squares). The rate of loss of CD6 is calculated by taking the slope between 10 and 120 minutes of each of the lines. FIG. 5A has plotted data points following treatment of CD4+CD45RA+ cells with 1.0 ug/mL isotype and the calculated slopes are 0.00 and −0.01 for antibody bound receptor and unbound receptor respectively. FIG. 5B has plotted data points following treatment of CD4+CD45RA+ cells with 0.1 ug/mL itolizumab and the calculated slopes are −0.05 and −0.18 for the antibody bound receptor and unbound receptor respectively. FIG. 5C has plotted data points following treatment of CD4+CD45RA+ cells with 1.0 ug/mL itolizumab and the calculated slopes are −0.20 and −0.33 for the antibody bound receptor and unbound receptor respectively. FIG. 5D has plotted data points following treatment of CD4+CD45RA− cells with 1.0 ug/mL isotype and the calculated slopes are 0.00 and −0.03 for antibody bound receptor and unbound receptor respectively. FIG. 5E has plotted data points following treatment of CD4+CD45RA− cells with 0.1 ug/mL itolizumab and the calculated slopes are −0.09 and −0.30 for the antibody bound receptor and unbound receptor respectively. FIG. 5F has plotted data points following treatment of CD4+CD45RA− cells with 1.0 ug/mL itolizumab and the calculated slopes are −0.36 and −0.49 for the antibody bound receptor and unbound receptor respectively.

Receptor occupancy of itolizumab is lower on cells with lower density and amount of cell surface CD6 as shown in FIGS. 6A-6D. FIG. 6A shows that CD8 cells have low levels of cell surface CD6 relative to CD4 cells, and FIG. 6B shows that itolizumab binds to CD8 cells with much lower occupancy than it does to CD4 cells, as measured by flow cytometry (cells were all incubated and stained within the same tube). Here, if itolizumab were to bind to CD6 regardless of cell surface density, then % occupancy (proportion of CD6 molecules bound by itolizumab) should be the same or higher in CD8 cells. However, the % occupancy in CD8 T cells is lower than in CD4 T cells, evidencing that itolizumab binding is affected by cell surface CD6 density.

FIG. 6C shows that occupancy is low in dosed patients. The dotted line shows blood at pre-dose day one baseline after being spiked with 50 ug/ml of itolizumab to determine highest possible receptor occupancy in each patient. CD6 mean fluorescent intensity on CD4 T cells at Day 1 is high but decreases within 24 hours of first dose, demonstrating a decrease in cell surface CD6. CD6 is still detectable on CD4 T cells after dosing (as indicated by MFI and gating against controls, squares); however, receptor occupancy (circles) essentially indicates little to no bound itolizumab. Some level of occupancy would be expected if itolizumab is able to bind CD6 regardless of receptor density. FIG. 6D shows that occupancy is detected when CD6 is present. Whole blood from normal subjects was collected into tubes containing EDTA or sodium citrate and incubated with itolizumab for 25 minutes. Blood was then lysed and cells assayed for receptor occupancy.

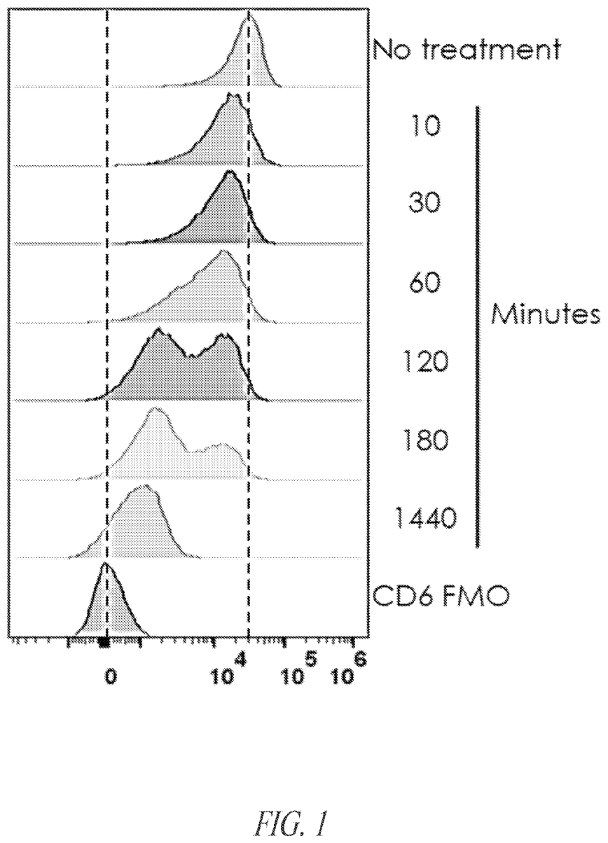

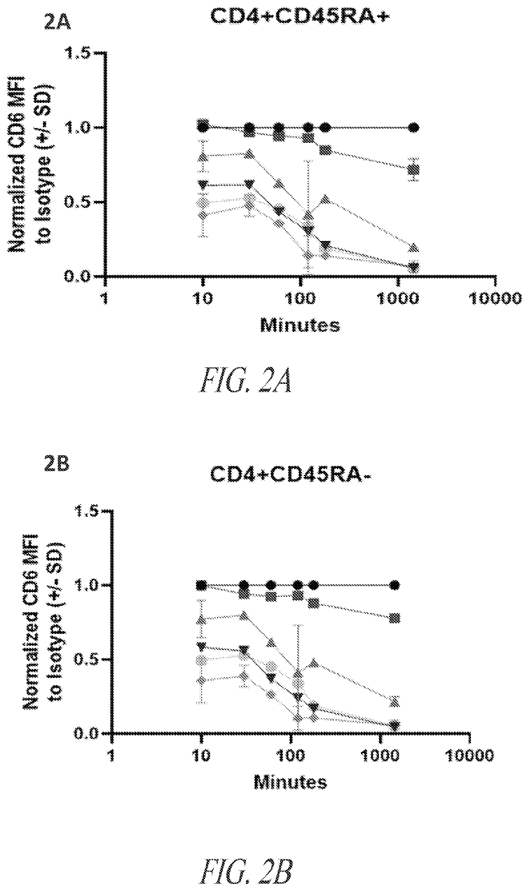

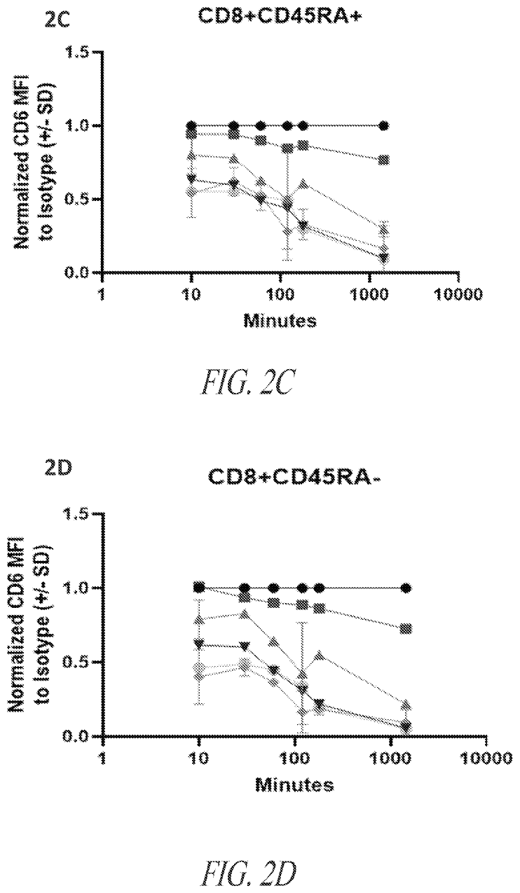

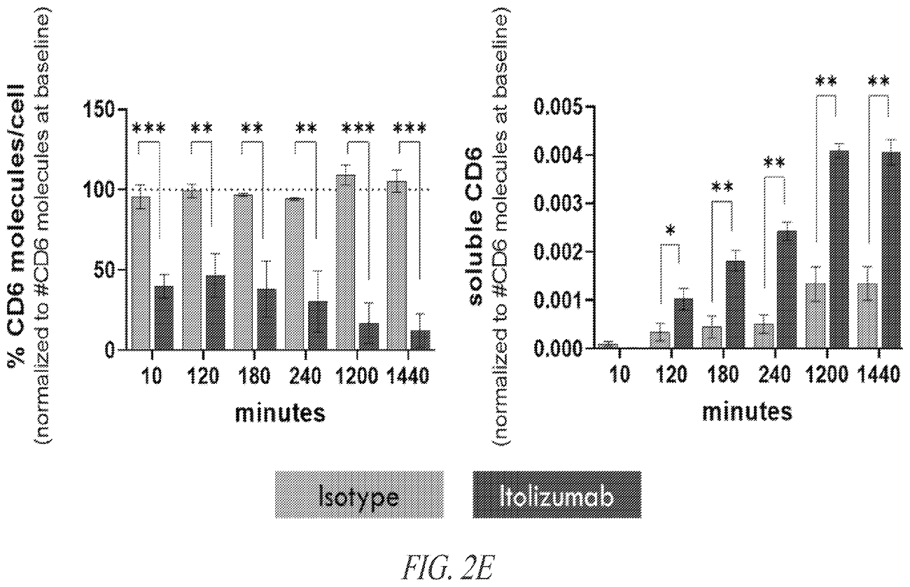

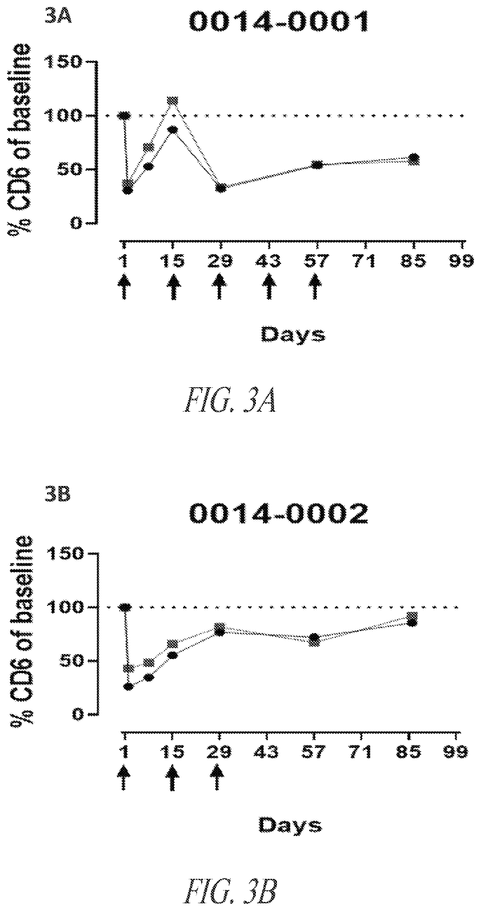

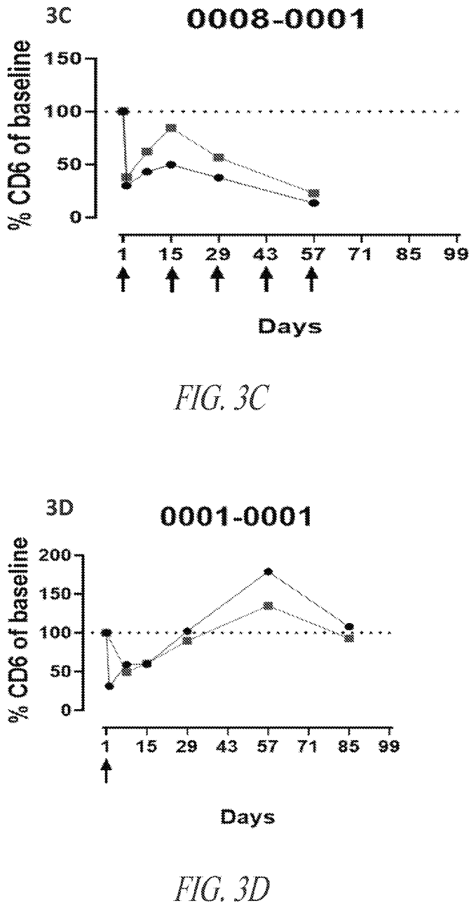

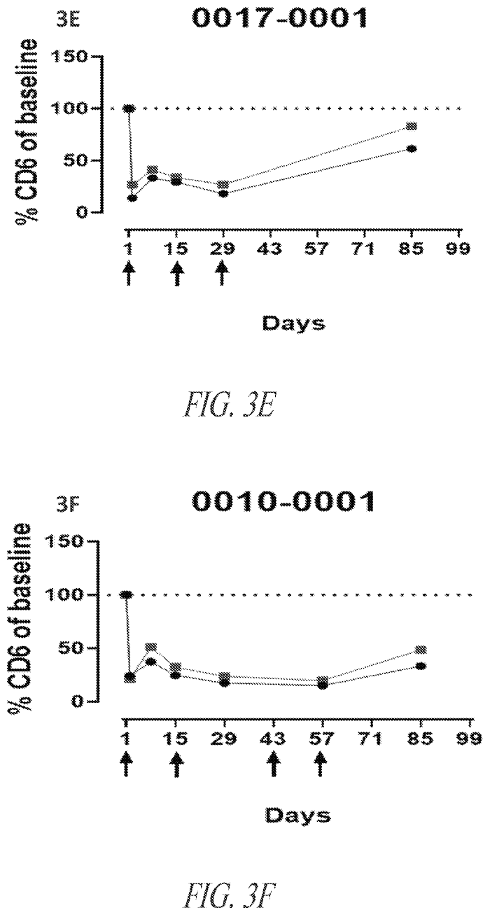

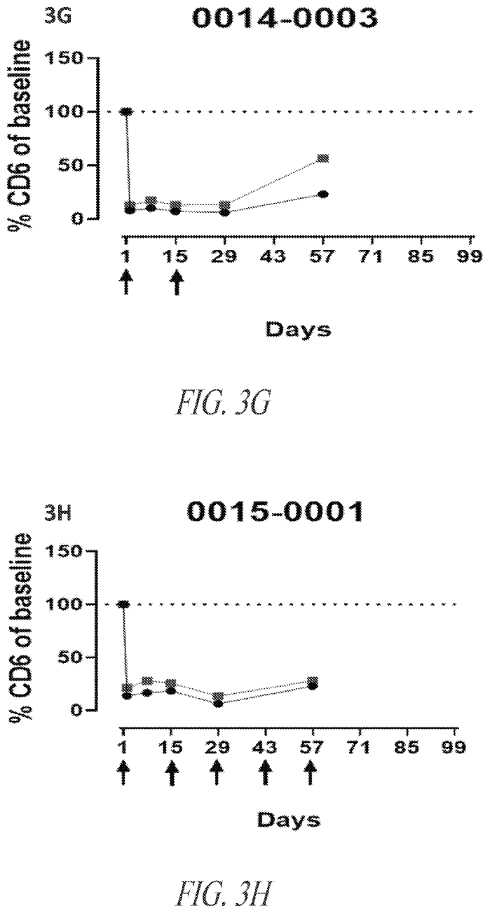

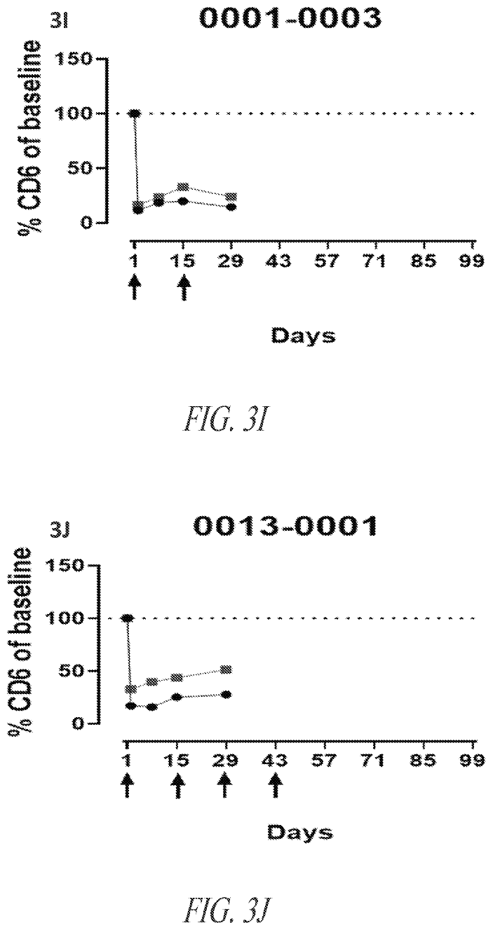

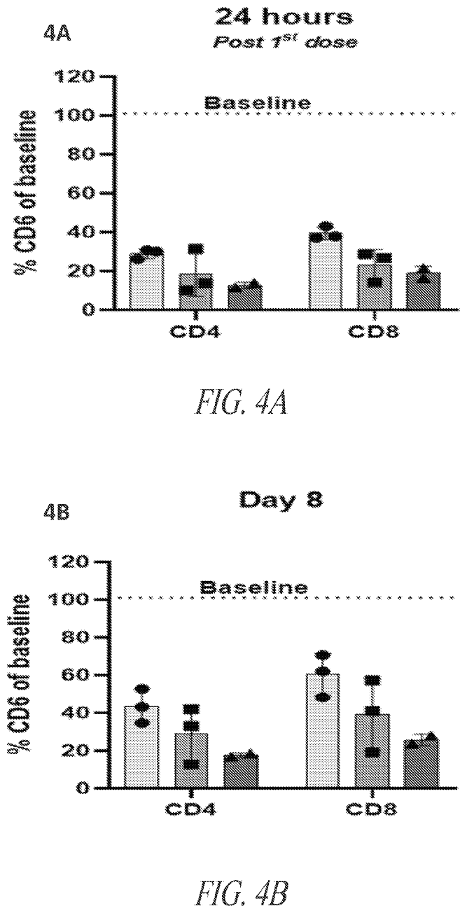

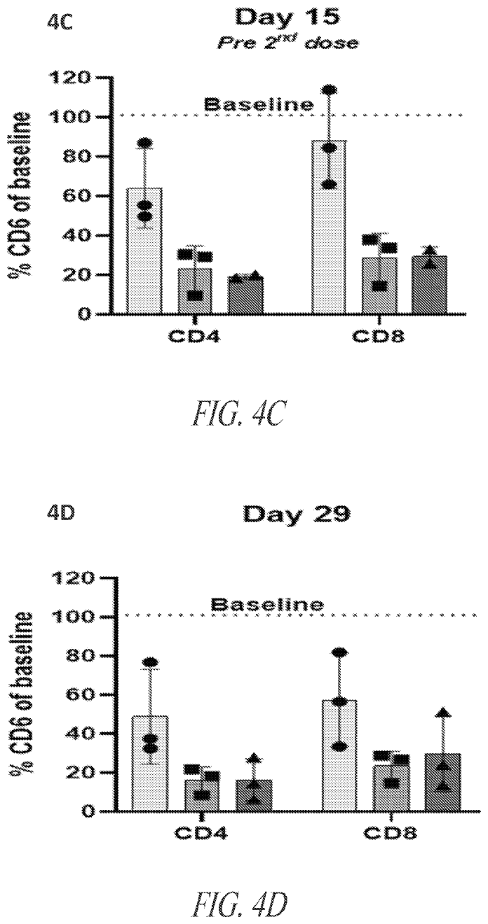

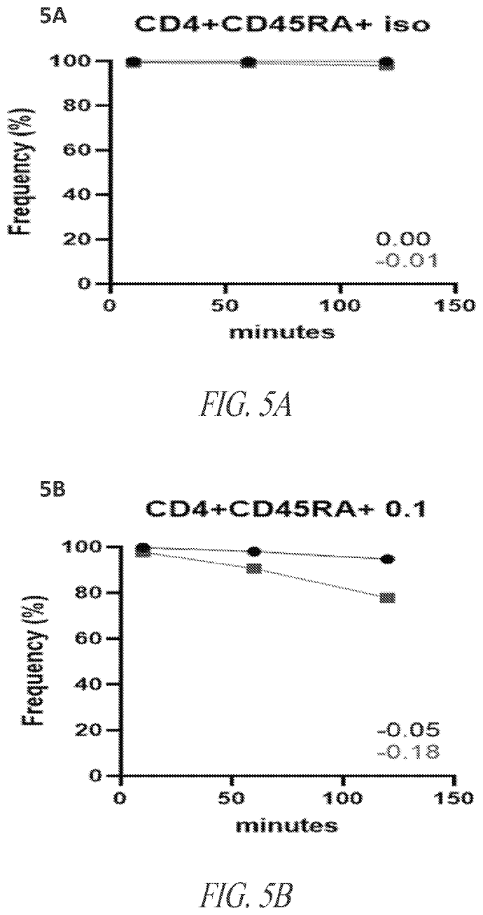

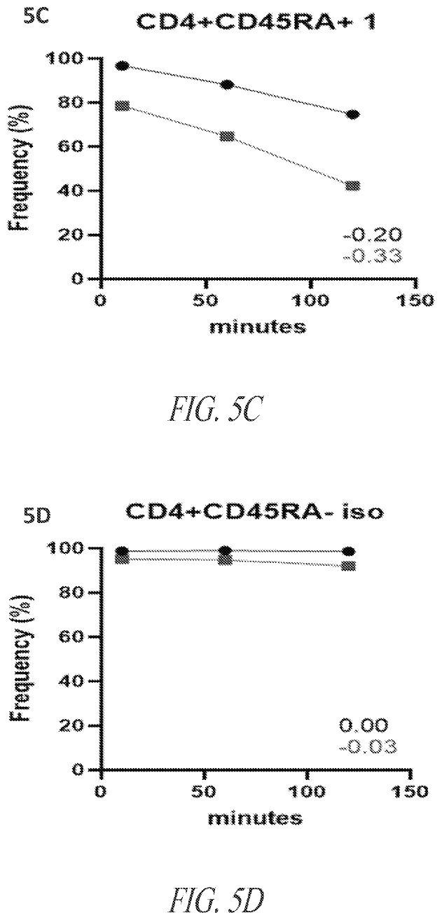

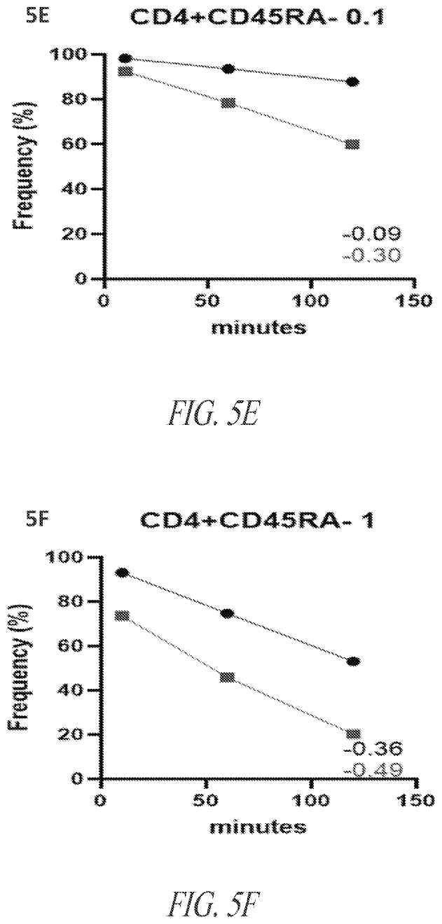

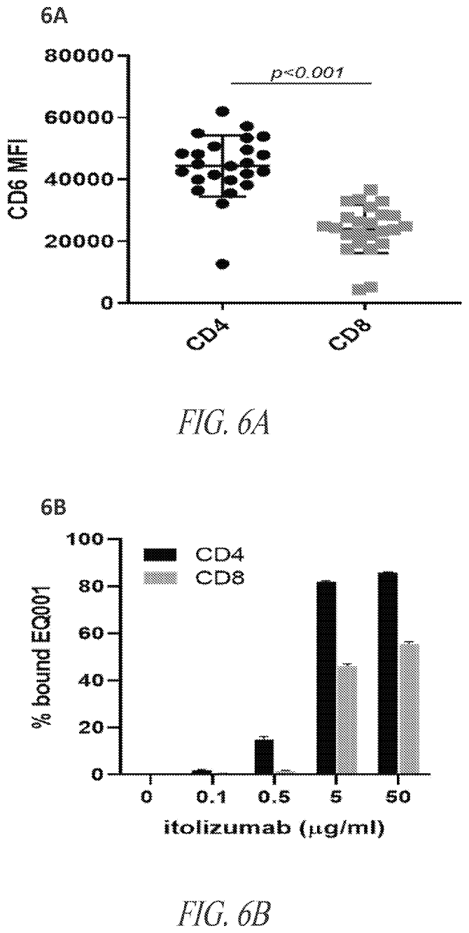

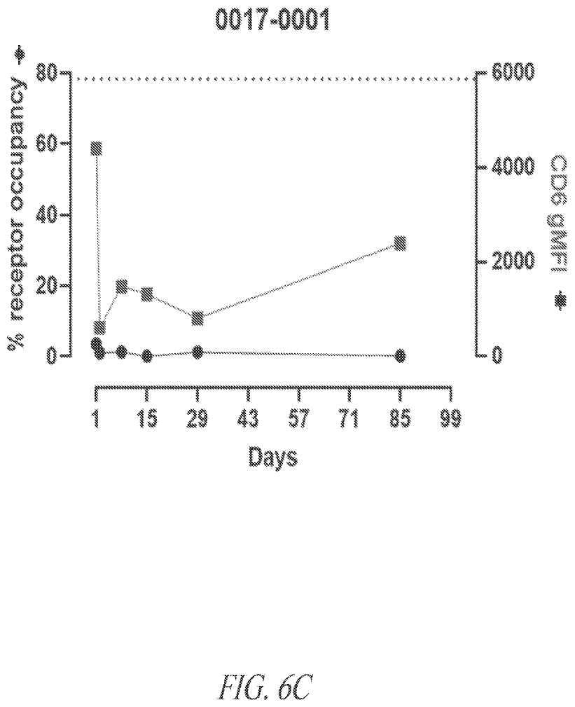

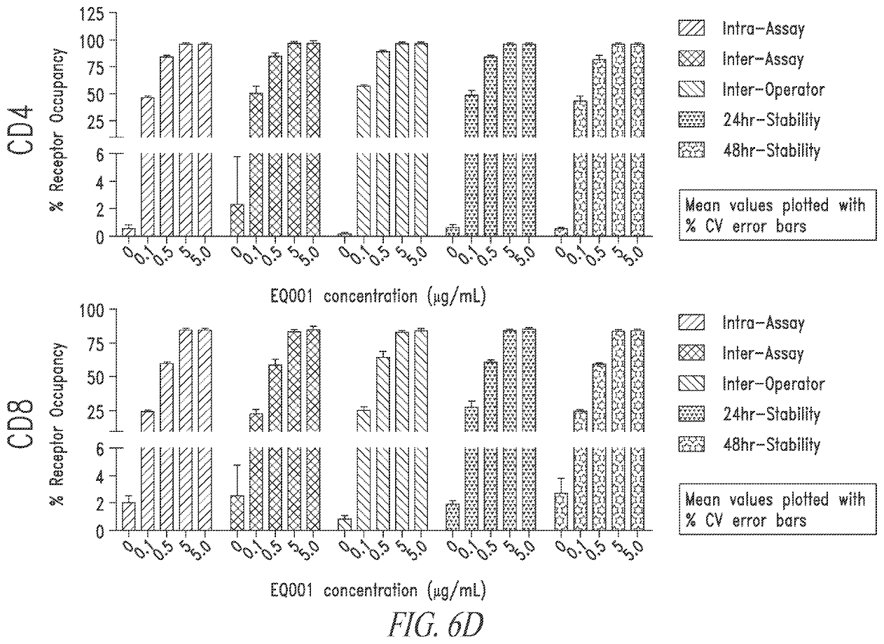

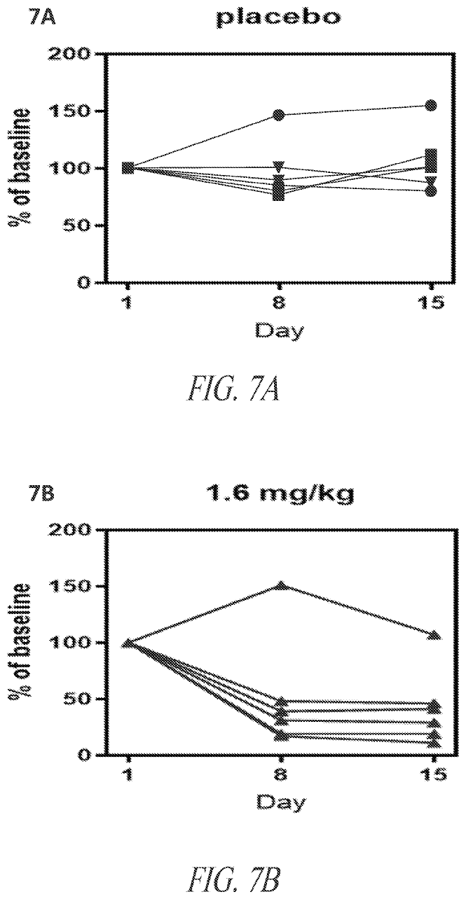

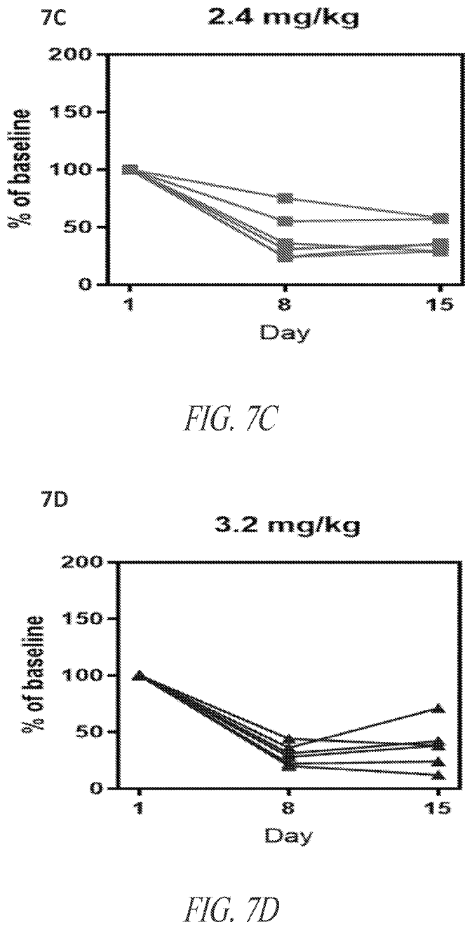

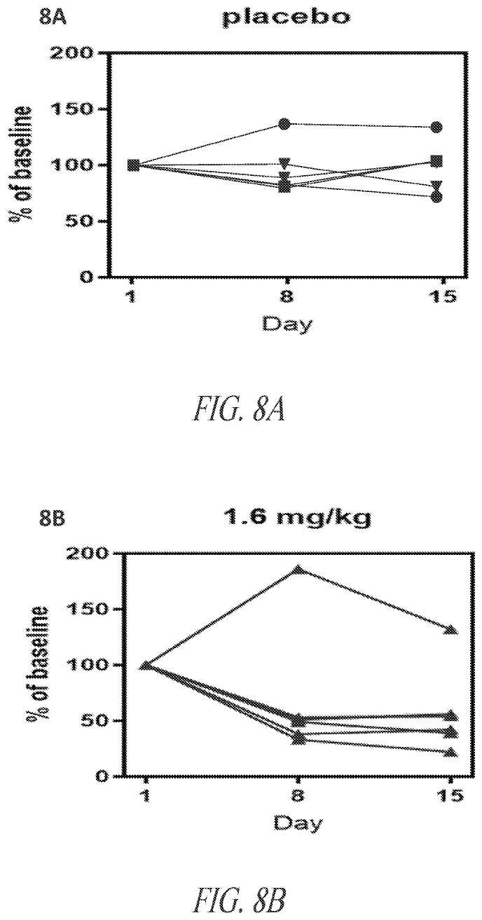

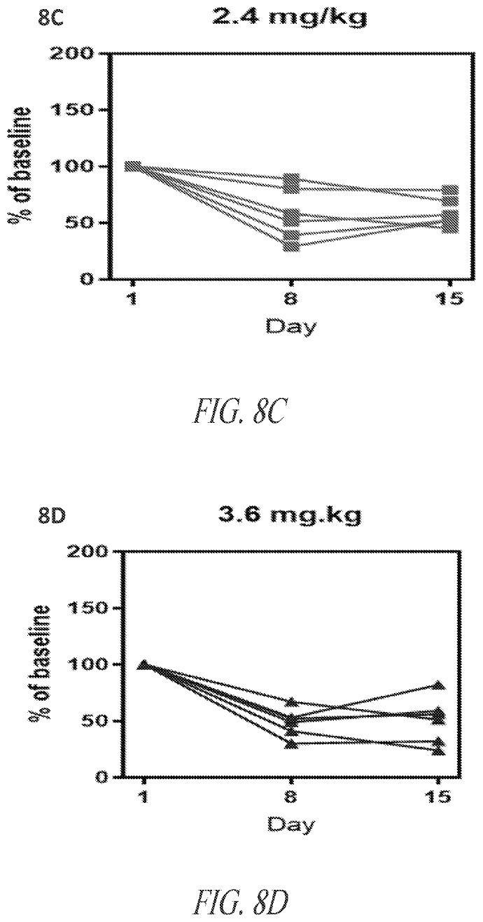

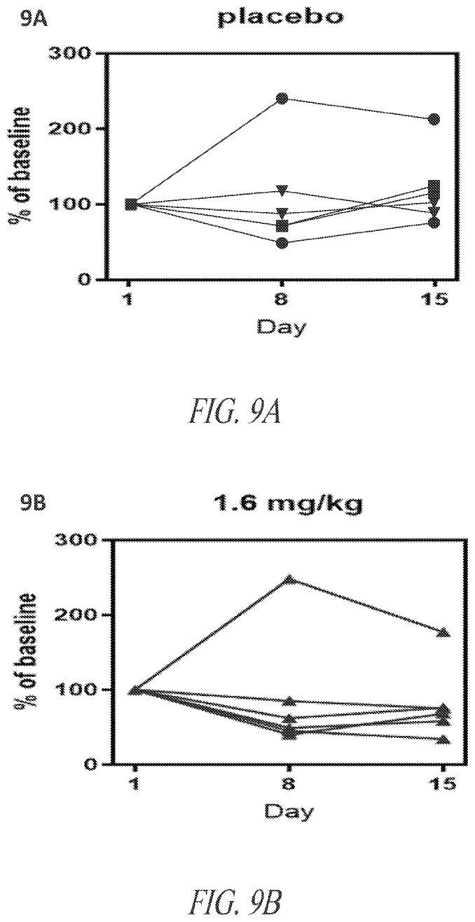

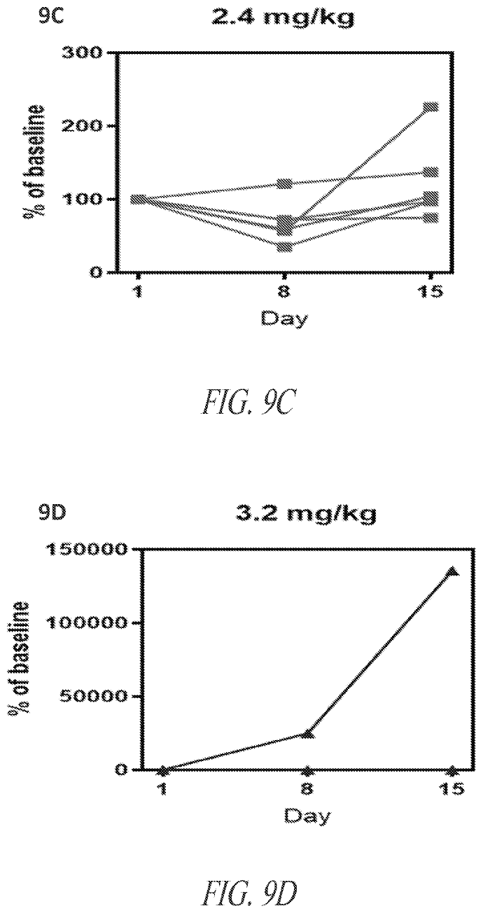

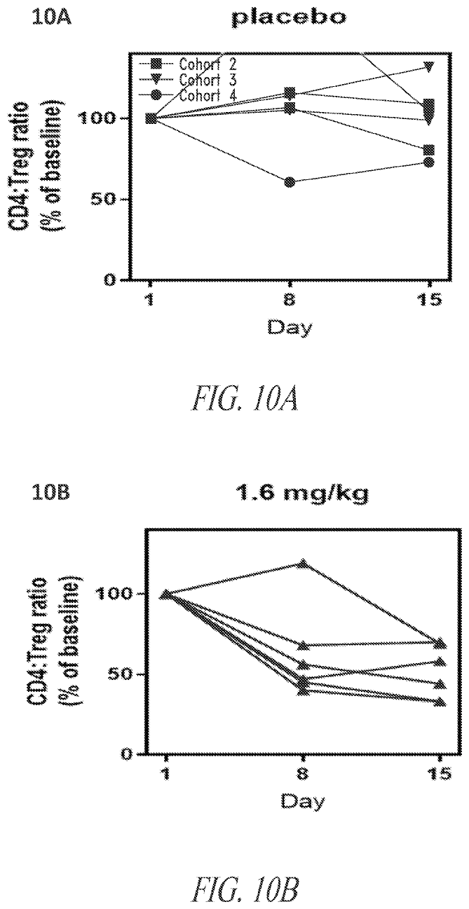

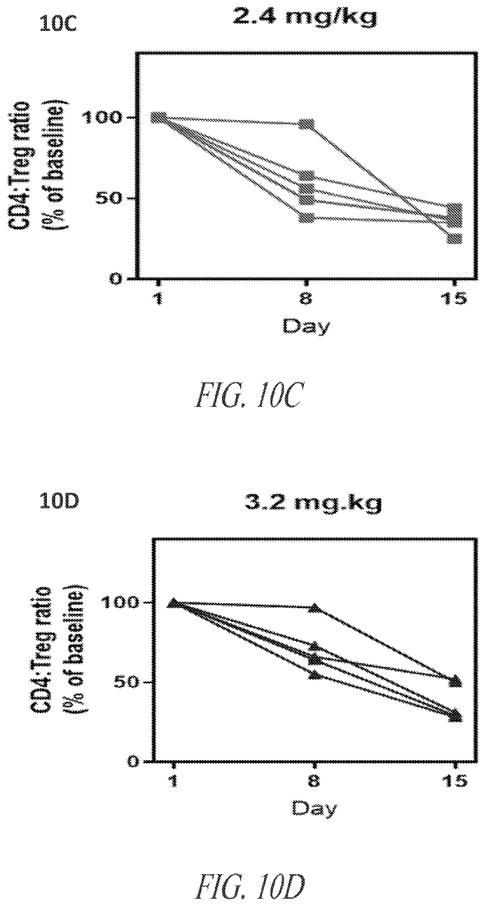

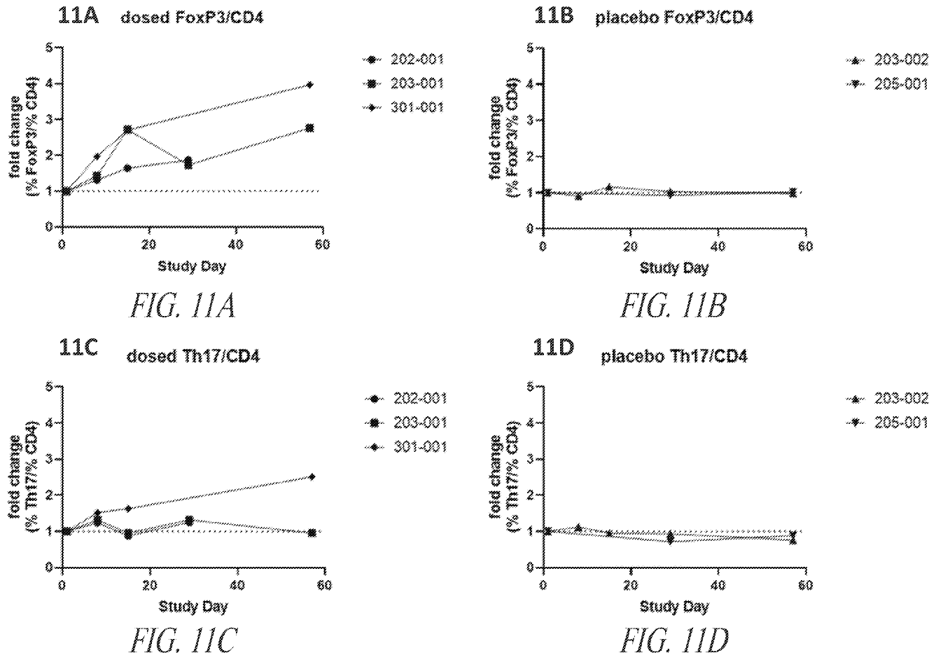

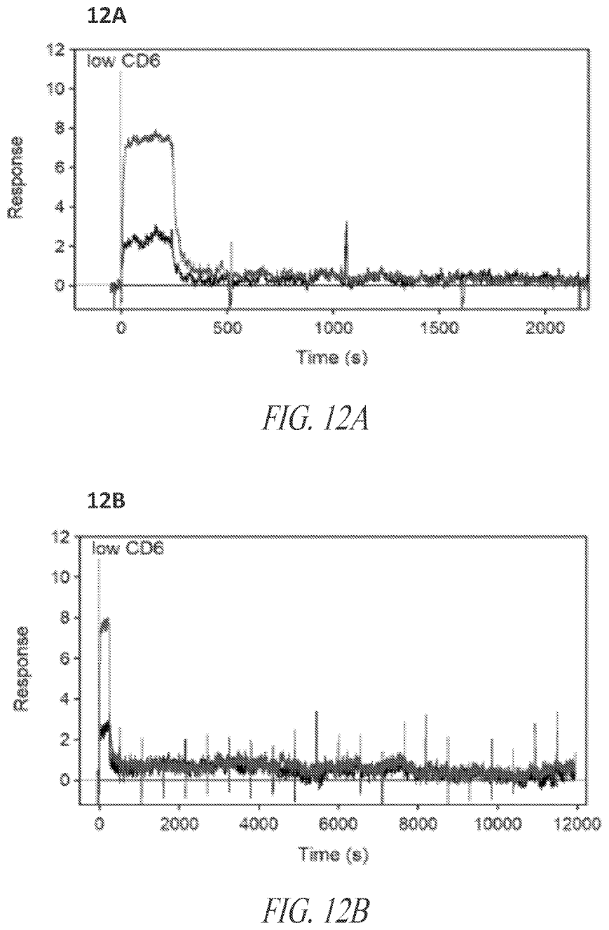

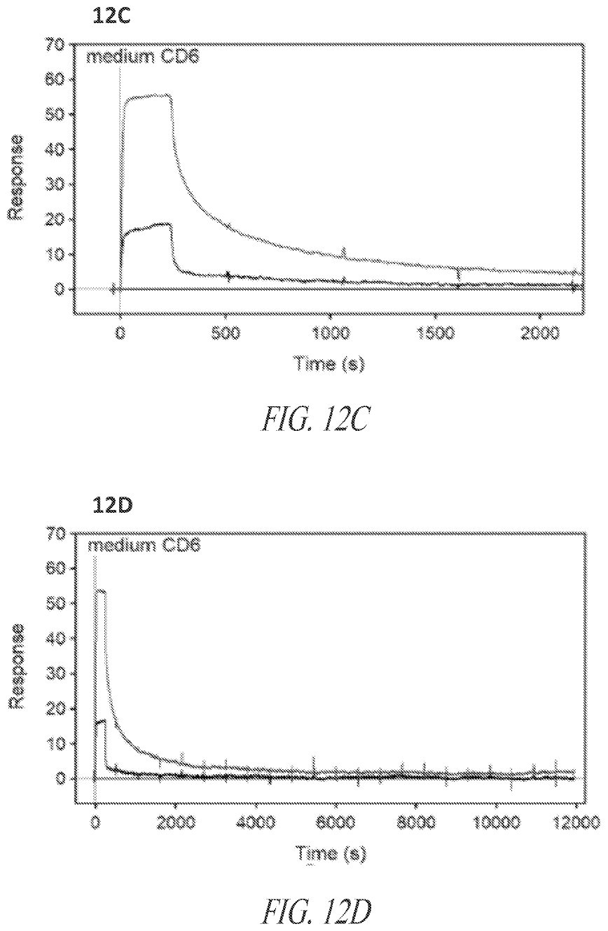

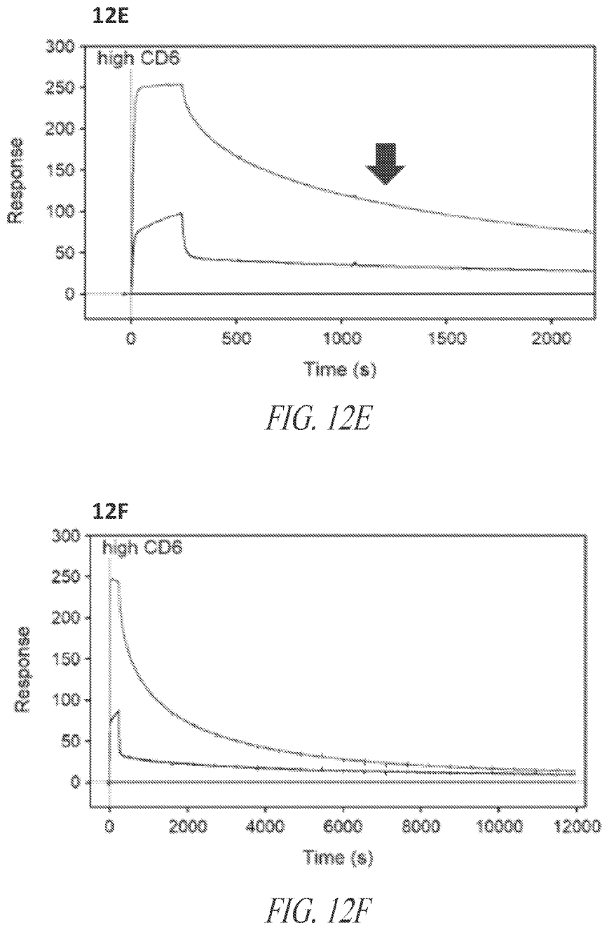

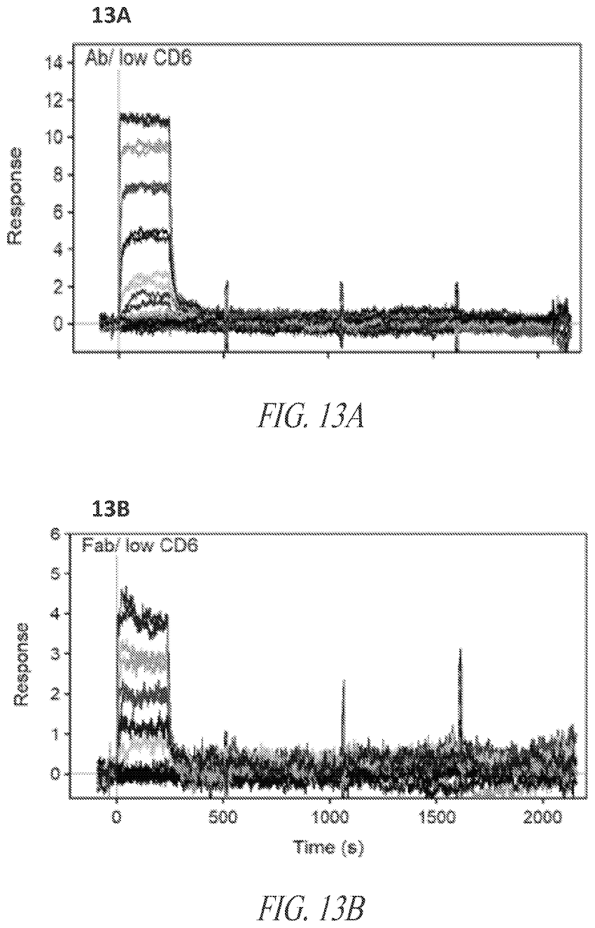

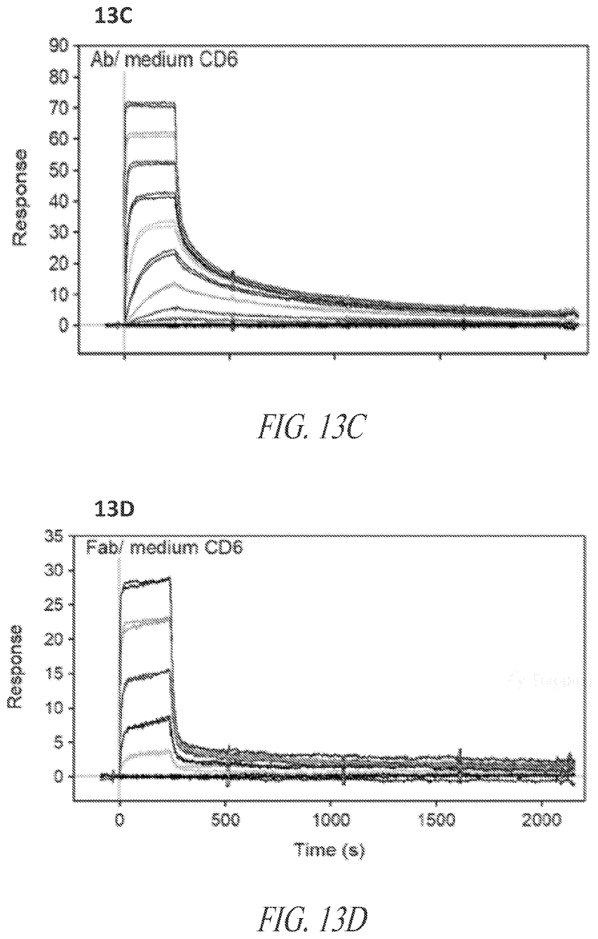

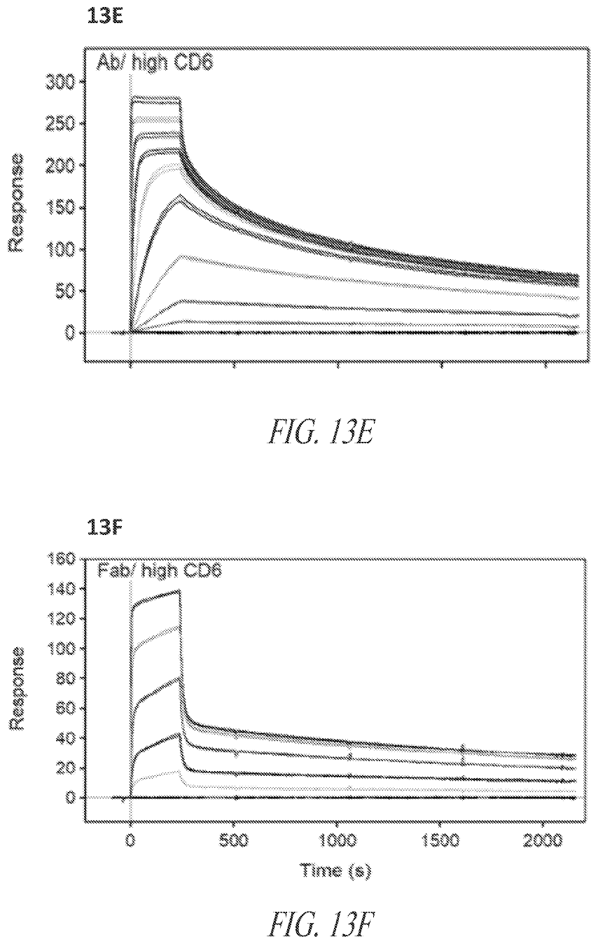

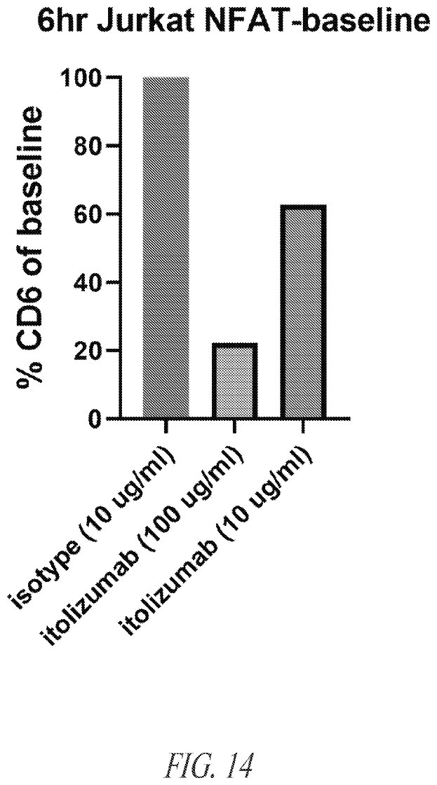

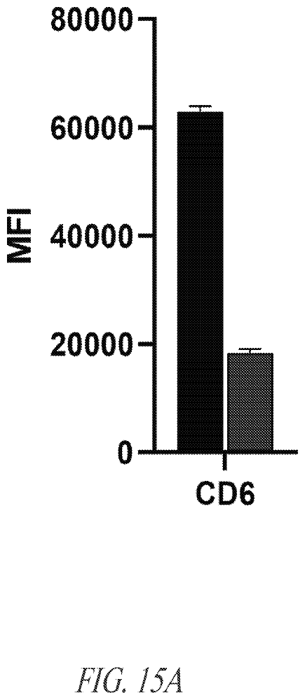

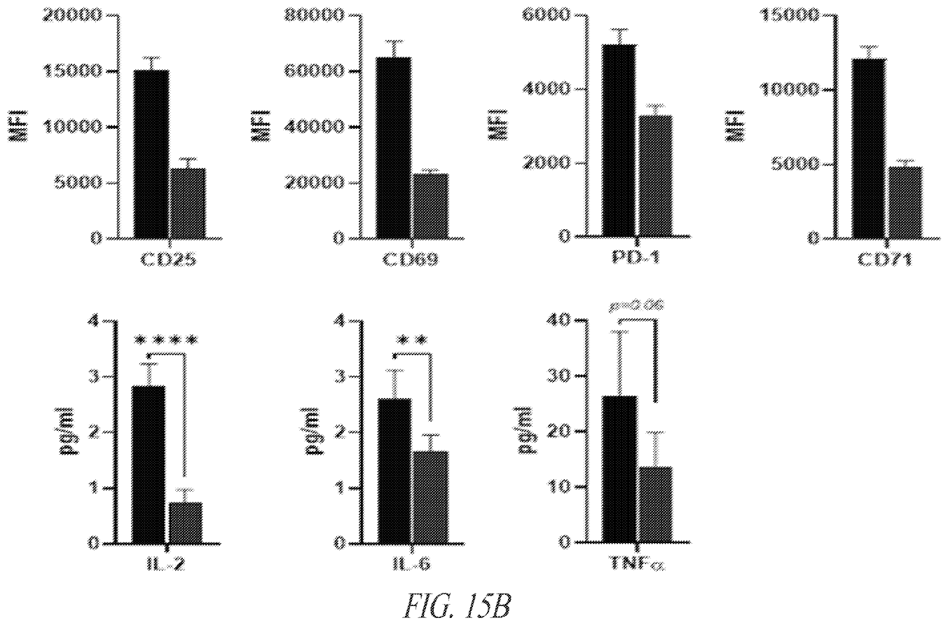

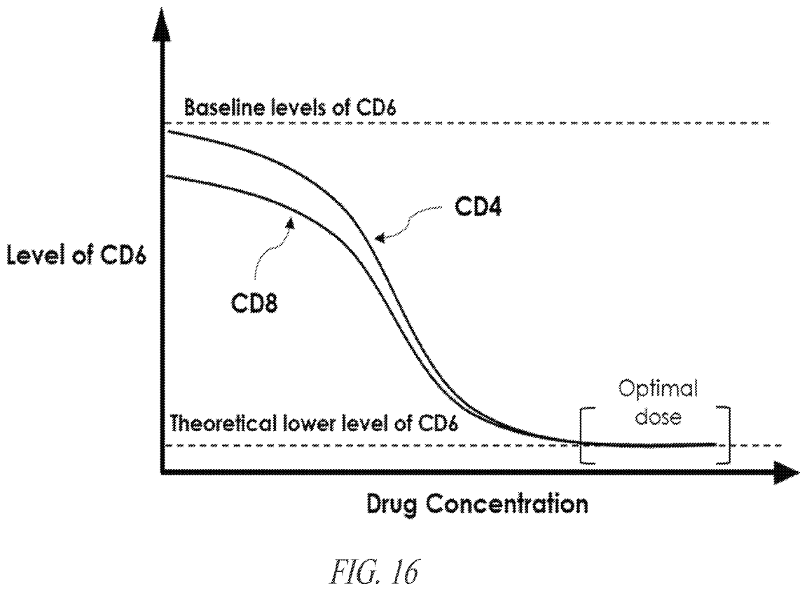

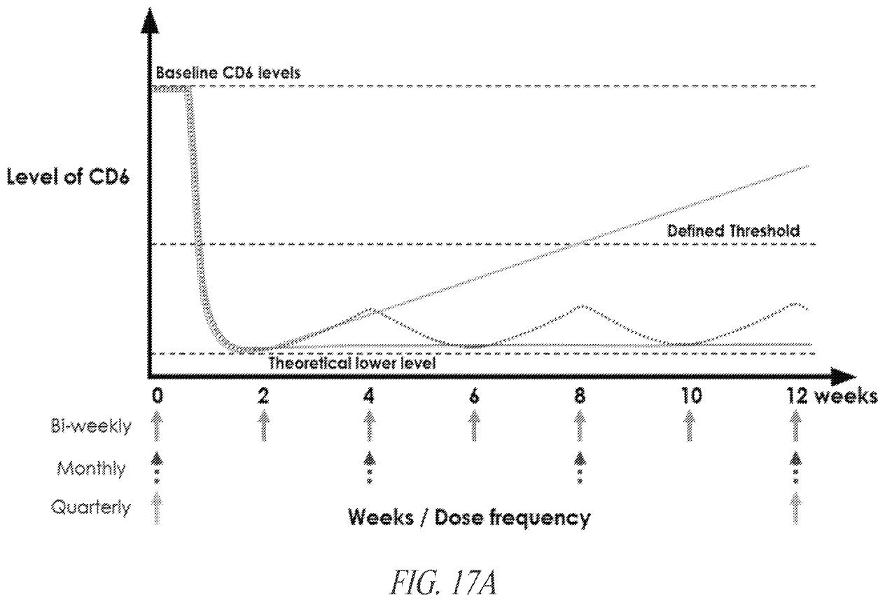

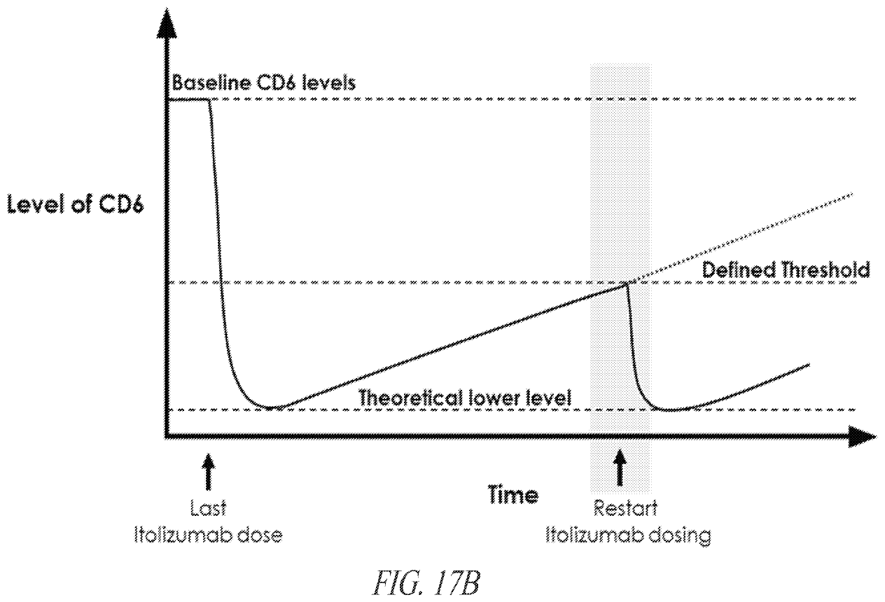

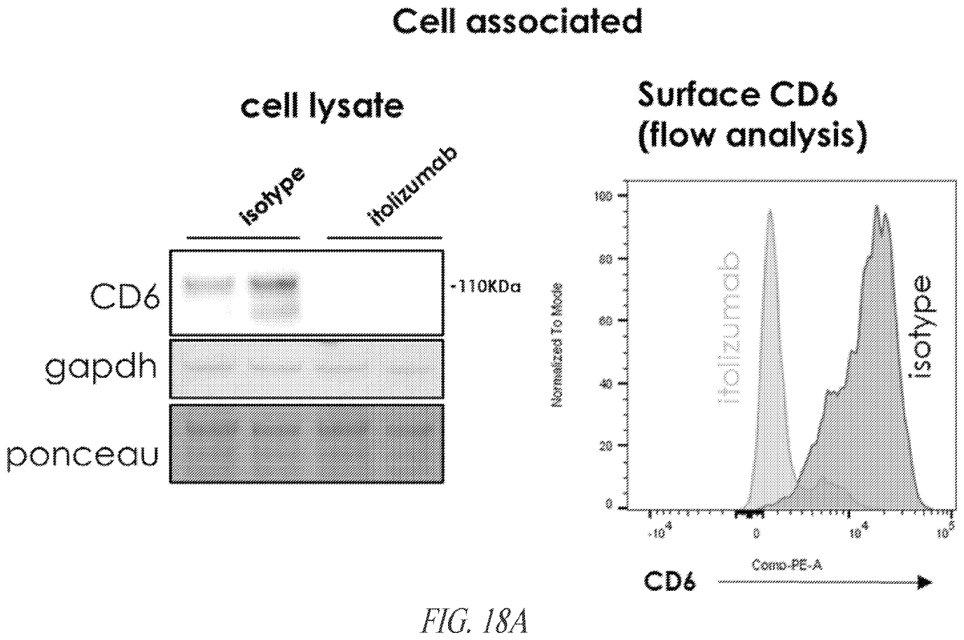

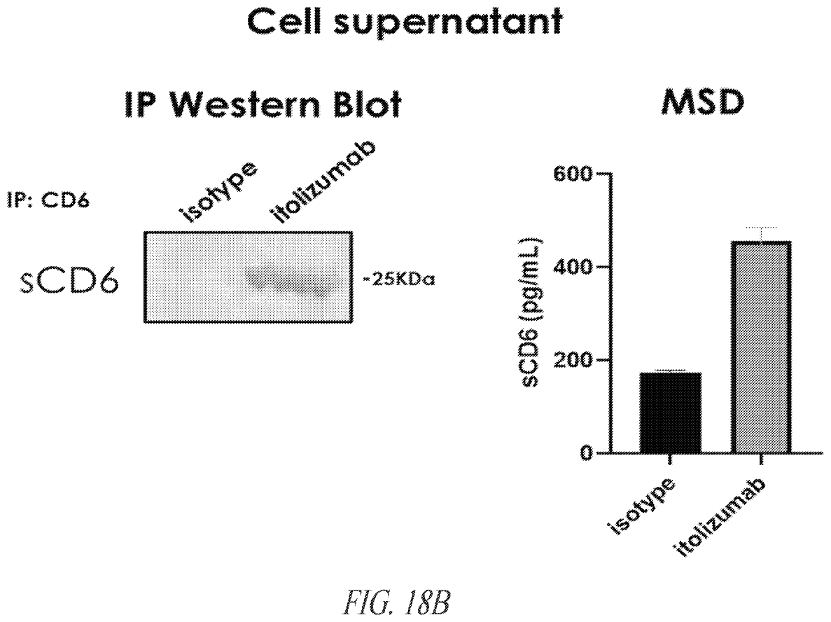

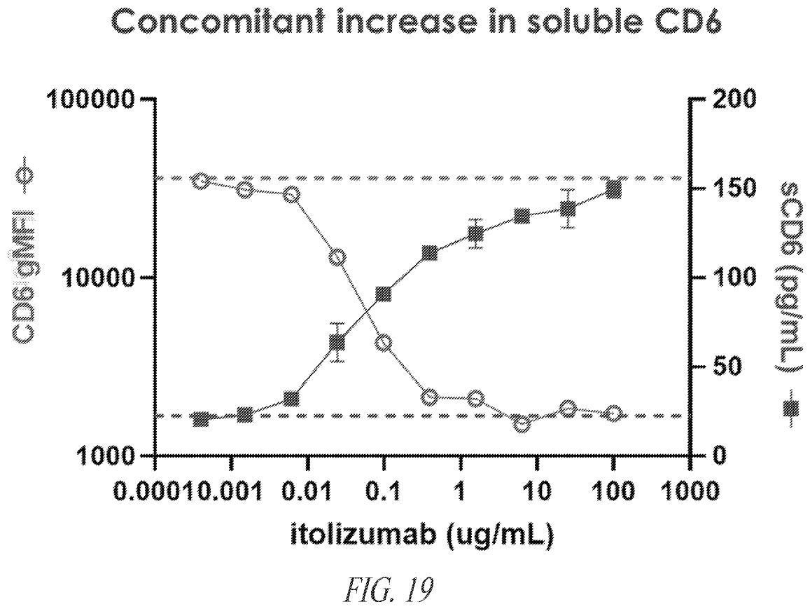

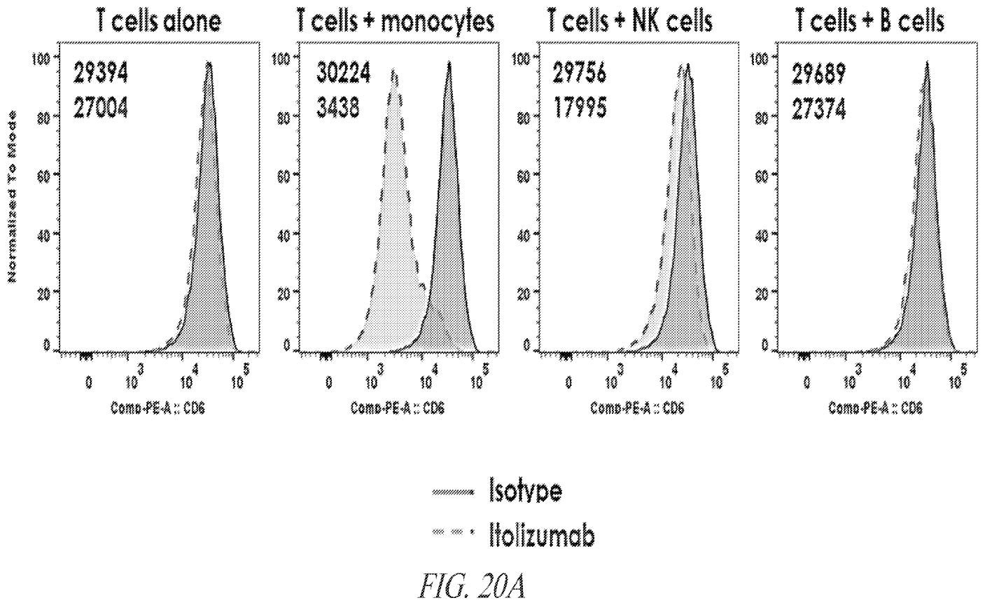

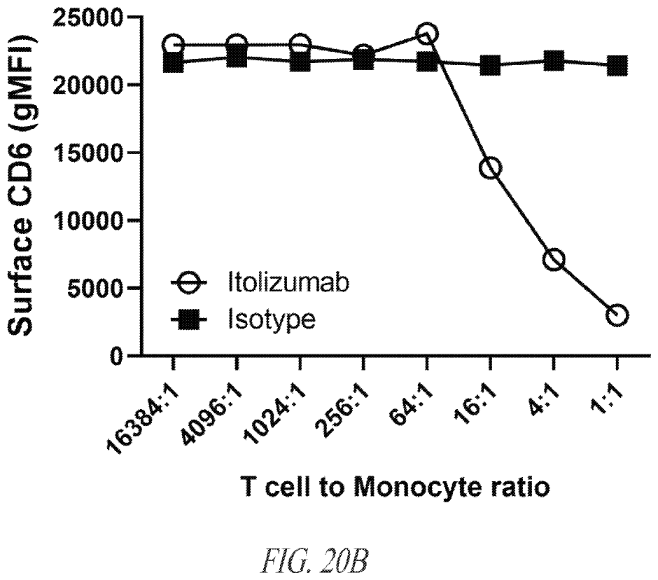

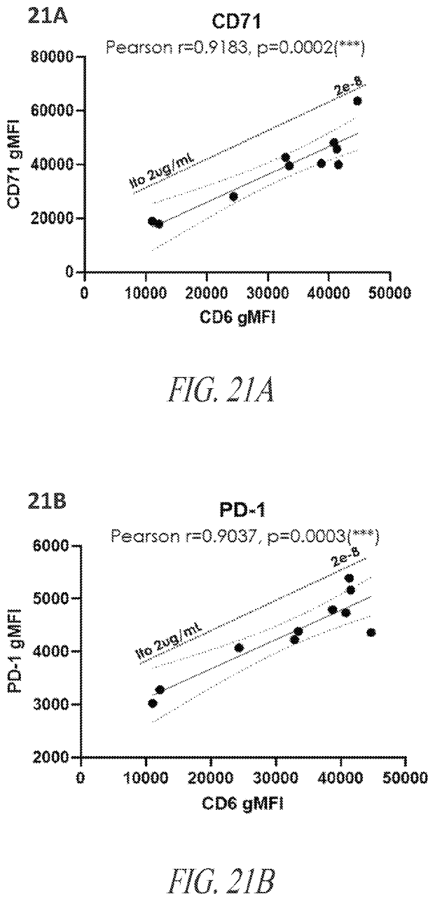

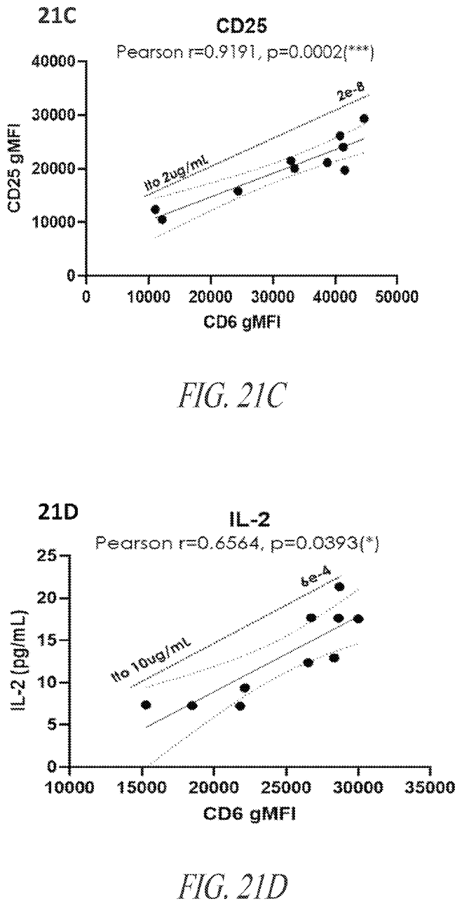

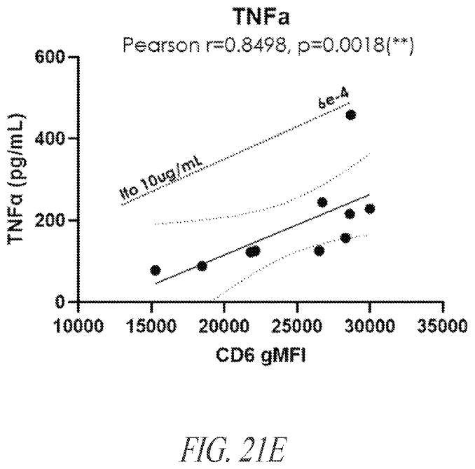

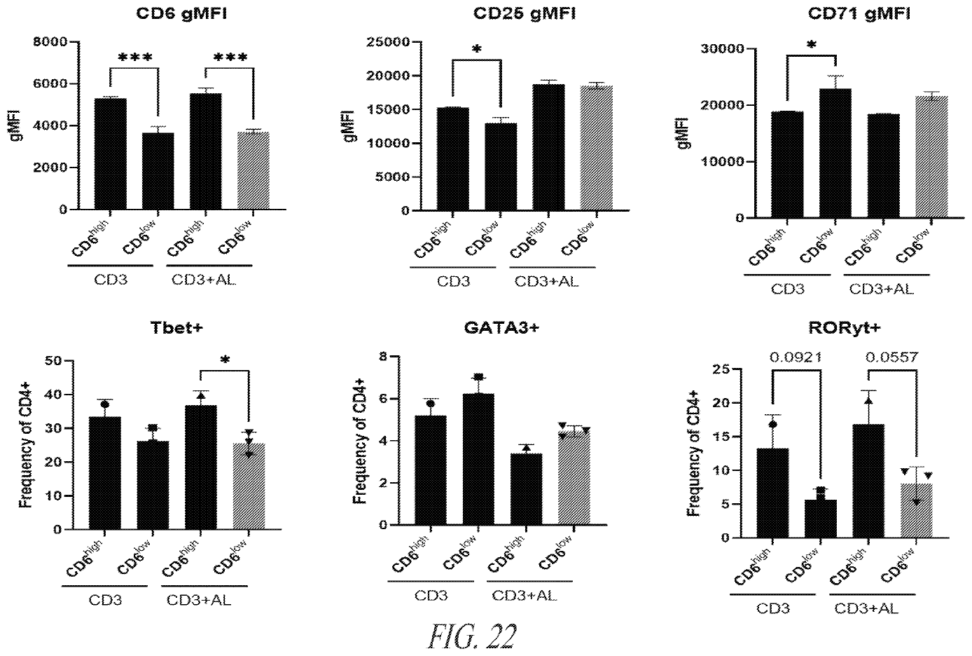

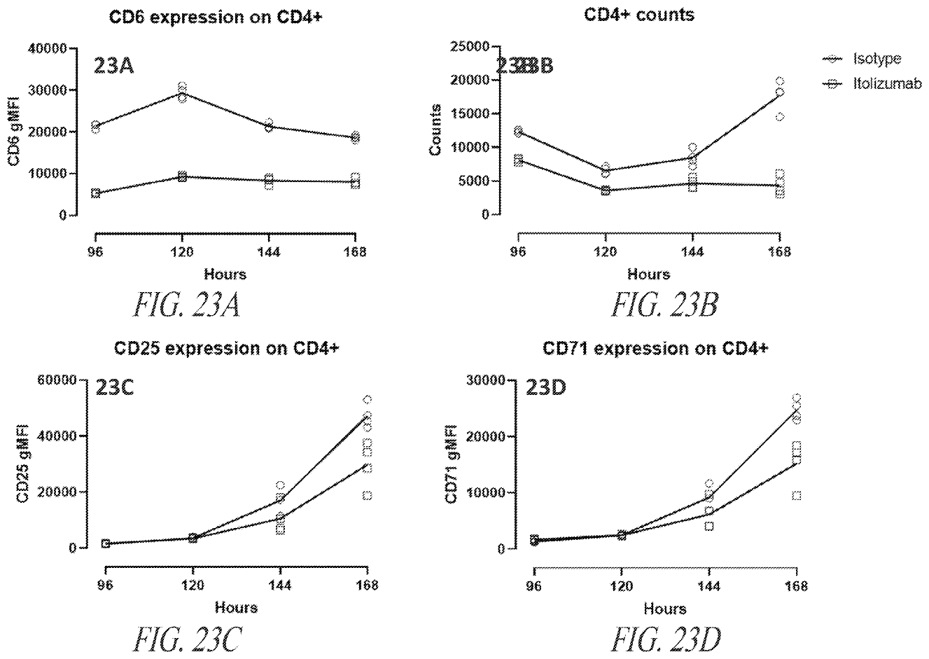

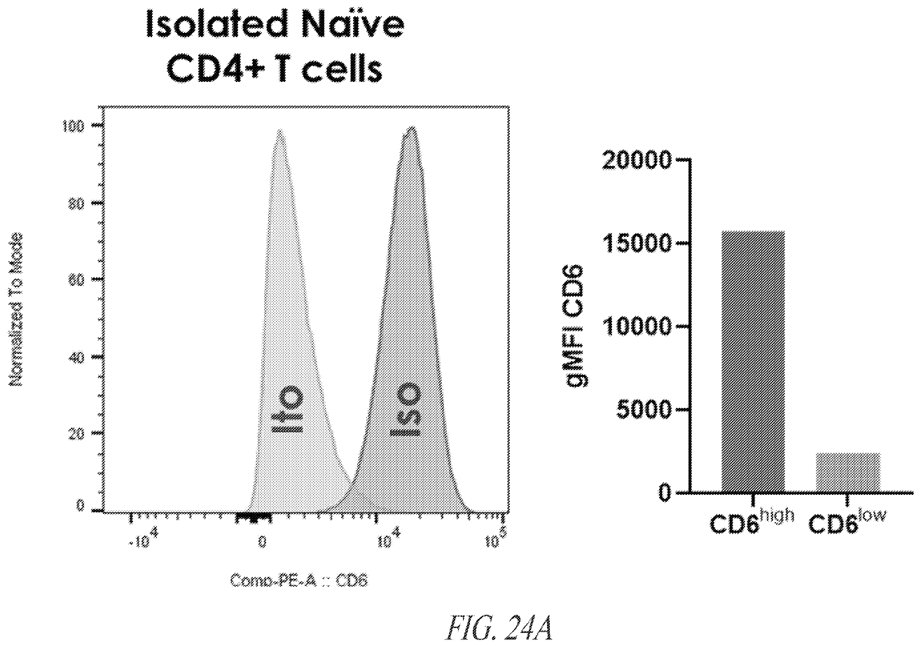

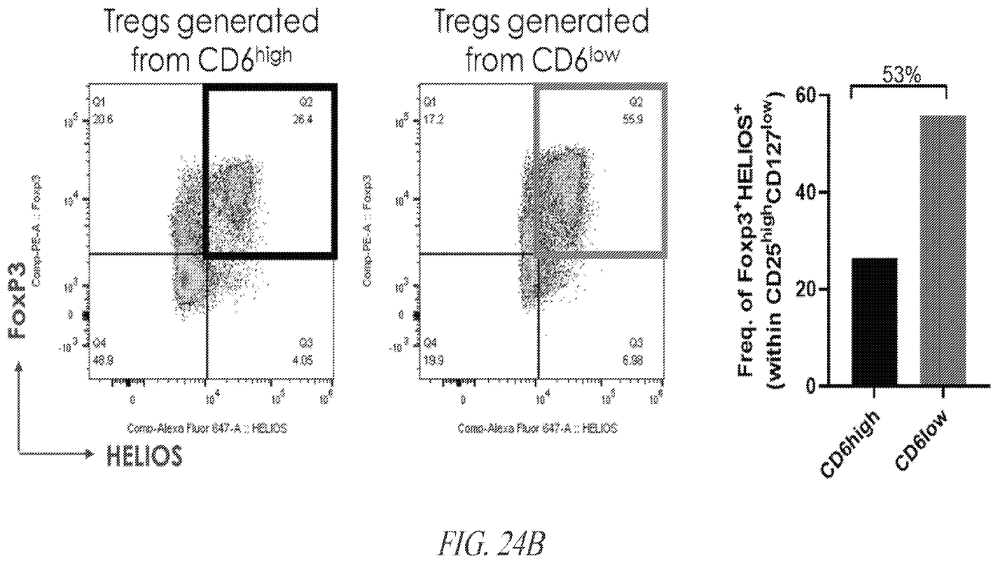

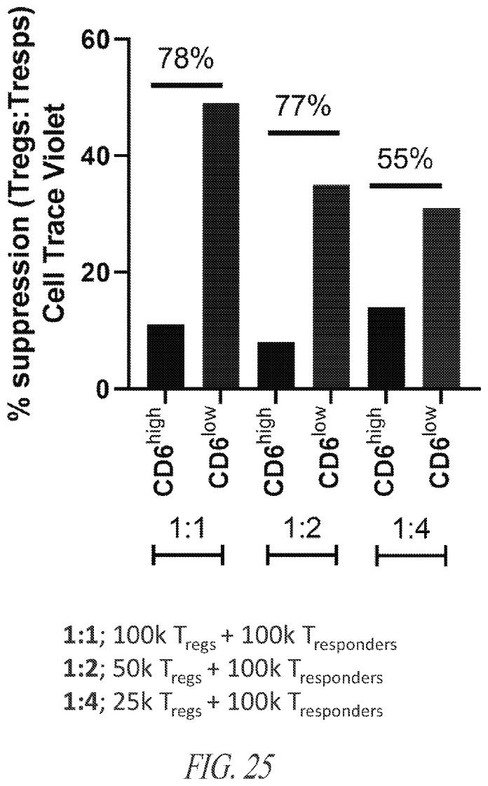

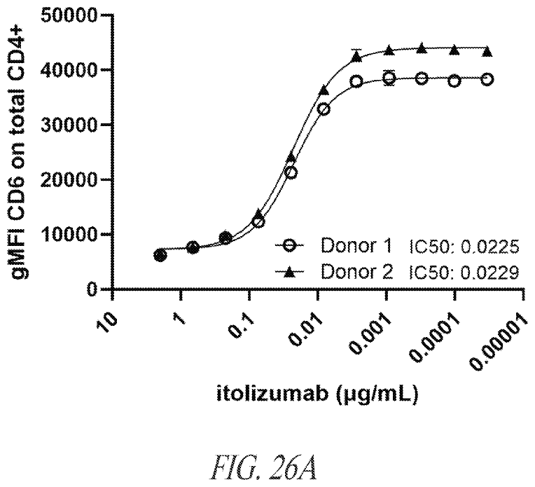

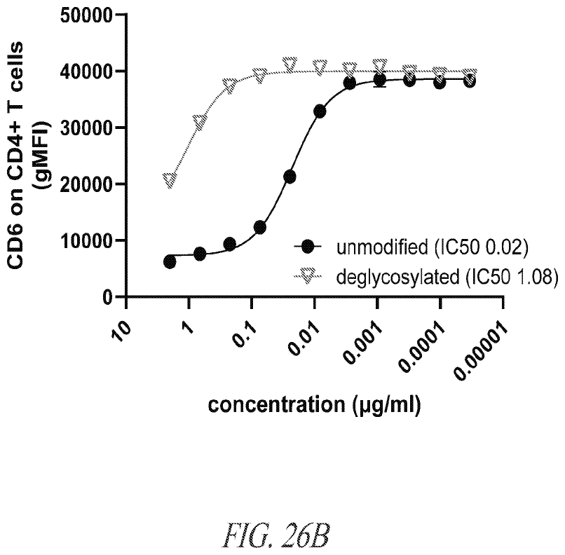

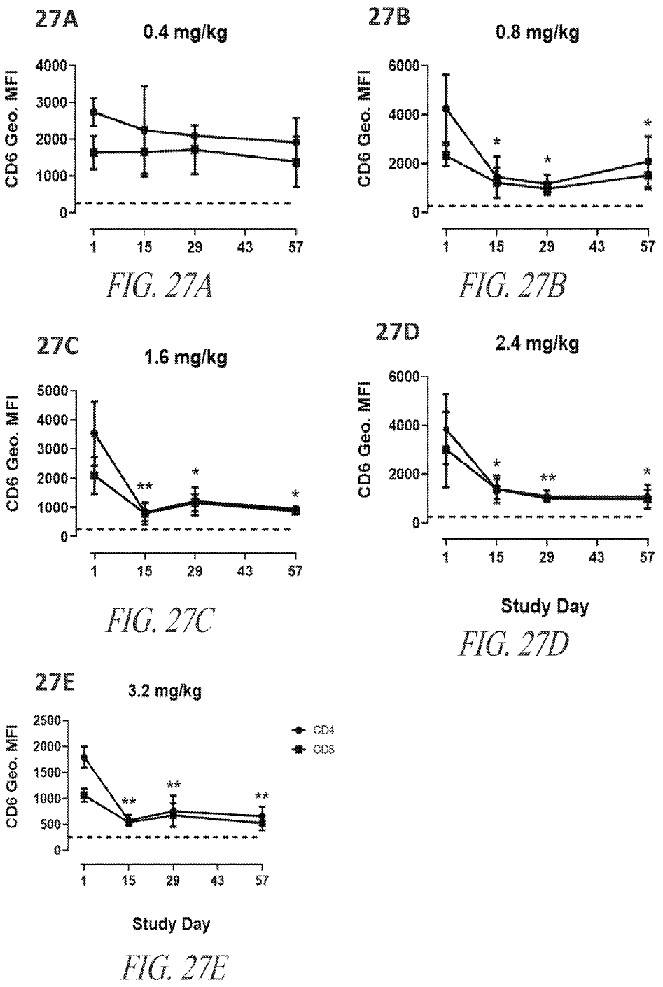

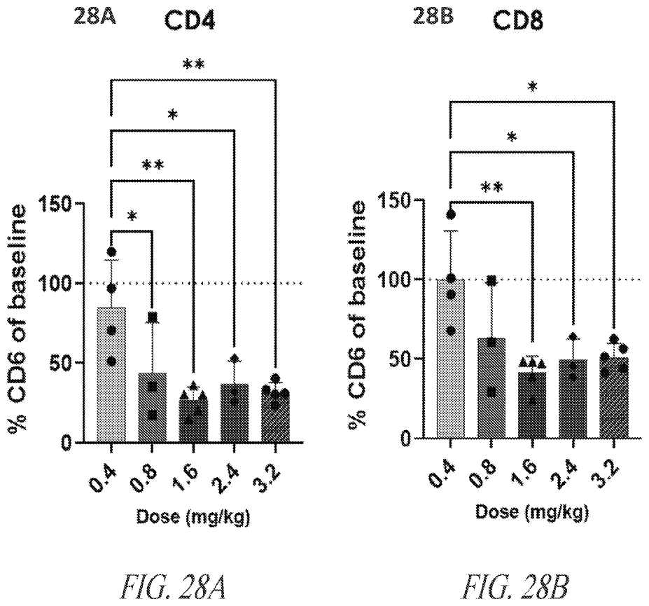

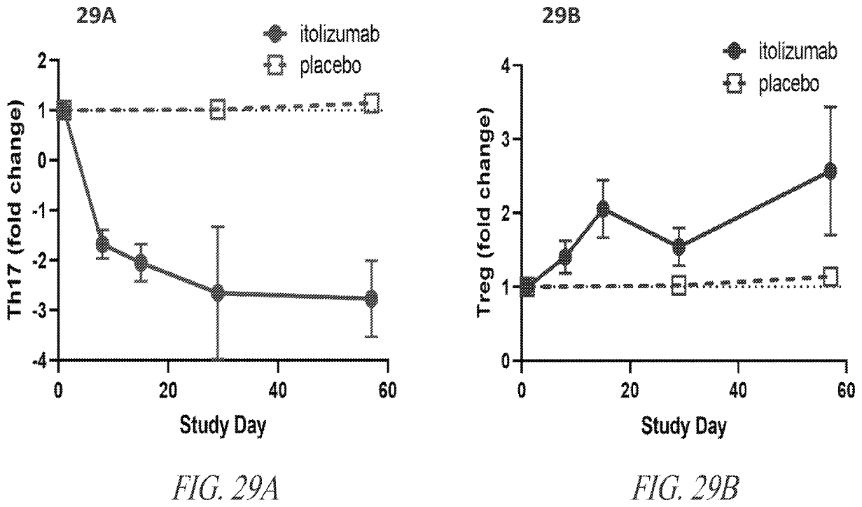

Itolizumab induces peripheral reduction of CD4 T cells. FIGS. 7A-7D show the peripheral reduction of CD4+ T cells in patients following treatment with itolizumab by measuring CD4+ cells from a peripheral blood draw and calculating as a percent of baseline for 6 different patients for four different conditions: placebo and 1.6, 2.4, and 3.6 mg/kg of itolizumab. FIG. 7A shows the change in percent baseline, with baseline established day 1, on days 8 and 15 following treatment with placebo. FIG. 7B shows the change in percent baseline, with baseline established day 1, on days 8 and 15 following treatment with 1.6 mg/kg itolizumab. FIG. 7C shows the change in percent baseline, with baseline established day 1, on days 8 and 15 following treatment with 2.4 mg/kg itolizumab. FIG. 7D shows the change in percent baseline, with baseline established day 1, on days 8 and 15 following treatment with 3.2 mg/kg itolizumab.

Itolizumab induces peripheral reduction of CD8 T cells. FIGS. 8A-8D show the peripheral reduction of CD8+ T cells in patients following treatment with itolizumab by measuring CD8+ cells from a peripheral blood draw and calculating as a percent of baseline for 6 different patients for four different conditions: placebo and 1.6, 2.4, and 3.6 mg/kg of itolizumab. FIG. 8A shows the change in percent baseline, with baseline established day 1, on days 8 and 15 following treatment with placebo. FIG. 8B shows the change in percent baseline, with baseline established day 1, on days 8 and 15 following treatment with 1.6 mg/kg itolizumab. FIG. 8C shows the change in percent baseline, with baseline established day 1, on days 8 and 15 following treatment with 2.4 mg/kg itolizumab. FIG. 8D shows the change in percent baseline, with baseline established day 1, on days 8 and 15 following treatment with 3.2 mg/kg itolizumab.

Peripheral T_{regs} counts are unaffected by itolizumab treatment. FIGS. 9A-9D show the change at days 8 and 15 calculated as a percentage of day 1 (baseline) T_{reg} cell counts following four treatment conditions: placebo, 1.6 mg/kg itolizumab, 2.4 mg/kg itolizumab, and 3.2 mg/kg itolizumab. FIG. 9A shows the variability in T_{reg} cell counts of the control group treated with placebo. FIG. 9B shows the percentage change in baseline for 6 different patients following treatment with 1.6 mg/kg itolizumab at days 8 and 15. FIG. 9C shows the percentage change in baseline for 6 different patients following treatment with 2.4 mg/kg itolizumab at days 8 and 15. FIG. 9D shows the percentage change in baseline for 6 different patients following treatment with 3.2 mg/kg itolizumab at days 8 and 15.

Itolizumab causes decreasing ratio of CD4:T_{reg} cells. FIGS. 10A-10D show the change from day 1 baseline at days 8 and 15 in the ratio of CD4+ T cells to T_{reg} cells in 6 patients following four treatment conditions: placebo, 1.6 mg/kg itolizumab, 2.4 mg/kg itolizumab, and 3.2 mg/kg itolizumab. FIG. 10A shows the ratio of CD4:T_{reg} as a percentage of baseline following treatment with placebo. FIG. 10B shows the ratio of CD4:T_{reg} as a percentage of baseline following treatment with 1.6 mg/kg itolizumab. FIG. 10C shows the ratio of CD4:T_{reg} as a percentage of baseline following treatment with 2.4 mg/kg itolizumab. FIG. 10D shows the ratio of CD4:T_{reg} as a percentage of baseline following treatment with 3.2 mg/kg itolizumab.

Increase in ratio of T_{reg}:CD4 T cells in patients treated with itolizumab. FIGS. 11A-11D show the ratio of Tregs:CD4 and the ratio of Th17:CD4 cells in patients following treatment with 0.8 mg/kg itolizumab or placebo at days 1, 8, 15, 29, and 57. The data was collected by taking cryopreserved PBMCs from the indicated timepoints, isolating DNA and measuring cell type-specific epigenetic markers in DNA using Epiontis ID, a proprietary qPCR assay. FIG. 11A shows the T_{reg}:CD4 ratio following itolizumab treatment while FIG. 11B shows the same ratio following placebo treatment. FIG. 11C shows the Th17:CD4 ratio following itolizumab treatment while FIG. 11D shows the ratio following placebo treatment.

Itolizumab has a slower off rate of binding with a higher density of CD6. FIGS. 12A-12F show the off-rate time for itolizumab (upper line) and an itolizumab fab (lower line) for three different CD6 densities for surface plasmon resonance. Low density consists of 7 rhu CD6 and 120 binding sites per square micrometer. Medium density consists of 55 rhu CD6 and 783 binding sites per square micrometer. High density consists of 255 rhu CD6 and 3493 binding sites per square micrometer. FIG. 12A shows the off-rates for the low density chip over 2000 seconds. FIG. 12B shows the off-rates for the medium density chip over 2000 seconds. FIG. 12C shows the off-rates for the high density chip over 2000 seconds. FIG. 12D shows the off-rates for the low density chip over 12000 seconds. FIG. 12E shows the off-rates for the medium density chip over 12000 seconds. FIG. 12F shows the off-rates for the high density chip over 12000 seconds.

More complex binding of itolizumab is observed at high densities of CD6. FIGS. 13A-13F show the complex binding of itolizumab to higher densities of CD6 compared to lower densities of CD6. The data were collected using 240 seconds association phases and 1800 seconds dissociation phases to three different CD6 densities. FIGS. 13A-13C are from duplicate injections of itolizumab concentrations ranging from 900 nM to 0.137 nM. FIGS. 13D-13F are from duplicate injections of itolizumab fab concentrations ranging from 900 nM to 11.1 nM. FIG. 13A shows the binding of different concentrations of itolizumab to the low density of CD6. FIG. 13B shows the binding of different concentrations of itolizumab to the medium density of CD6. FIG. 13C shows the binding of different concentrations of itolizumab to the high density of CD6. The sensorgrams for itolizumab binding to CD6 shows complex binding at the highest chip density of CD6. FIG. 13D shows the binding of different concentrations of itolizumab fab to the low density of CD6. FIG. 13E shows the binding of different concentrations of itolizumab fab to the medium density of CD6. FIG. 13F shows the binding of different concentrations of itolizumab fab to the high density of CD6. The sensorgrams for itolizumab fab binding to CD6 shows minimal complex binding at all three (low, medium, high) chip densities of CD6. These results demonstrate that itolizumab exhibits strong avidity effects that confer preferential binding to higher density of CD6 on the chip and thus support the observations of preferential binding and selectivity of itolizumab to CD6^{high} cells in vivo.

Itolizumab causes loss of cell-surface CD6 on a cell line. FIG. 14 shows that itolizumab decreases cell surface CD6 levels on a T cell line that expresses CD6. Here, Jurkat NFAT cells were thawed and resuspended at a concentration of 1×10⁶ cells/ml, and then aliquoted into single tubes at 1×10⁶ cells/ml. Itolizumab or isotype control antibodies were added, and the cells were gently and incubated for 6 hrs at 37° C. Total cell surface CD6 was assessed by flow cytometry (cells were kept at 4° C. during staining).

CD6^{low} cells are hyporesponsive to TCR stimulation. FIGS. 15A-15B show that CD6^{low} cells are hyporesponsive to stimulation. Here, PBMCs were incubated with itolizumab for 24 hours to remove cell surface CD6 (see FIG. 15A; CD6^{high} left column; CD6^{low} right column), washed at least 3 times to remove excess and bound itolizumab, and stimulated with 0.25 μg/ml anti-CD3 and 1 μg/ml ALCAM for 24 hours. Activation markers were assessed by flow cytometry and cytokines were assessed by flow-based ELISA. As shown in FIG. 15B, the CD6^{low} cells (right column in each graph) show a reduction in surface activation markers and cytokine release relative to CD6^{high} cells (left column in each graph), evidencing that itolizumab not only reduces cell surface CD6 levels in PBMCs, but also renders the cells less responsive to T cell activating factors such as CD3.

Diagram of dose selection based on CD6 expression. FIG. 16 illustrates the use of cell surface CD6 to inform dose selection of itolizumab. One approach is to dose a patient or a group of patients with increasing amounts of itolizumab. The lower level of CD6 is determined when no further decrease of CD6 levels is observed with addition of more drug by increased dose or by a subsequent dose at a fixed interval. Once the optimal dosage is determined that reduces the levels of CD6 to the lower level, the levels of CD6 can also be used to determine an optimal dosing regimen.

Diagram of dosing regimen based on CD6 expression. FIGS. 17A-17B illustrate the selection of a suitable dosing regimen. One approach is to monitor cell surface CD6 levels over time to determine how long any given dose provides sustained pharmacodynamic effect of loss of cell surface CD6. The exemplary graph of FIG. 17A illustrates that bi-weekly and monthly dosages would be suitable but that quarterly doses at that level would not be suitable because the cell surface CD6 levels cross the threshold at eight weeks. This information may indicate that more drug is required in order to achieve sustained loss of cell surface CD6 for quarterly dosing. In some instances, the upper limit or threshold is about, less than about, or between about 25-50% of the baseline level of cell surface CD6. FIG. 17B illustrates this approach in a short course of itolizumab therapy, where the levels of cell surface CD6 can be used to inform the need to restart dosing or the potential for a disease flare. After the initial dosing period is finished, cell surface levels of CD6 can be monitored over time, and once these levels rise to or approach a threshold relative to the baseline (e.g., pre-treatment levels of cell surface CD6), or relative to a defined threshold or target level (e.g., a clinically-suitable or desirable level of cell surface CD6, for example, as defined by other disease parameters or symptoms), a decision can be made to dose the patient again with itolizumab.

FIGS. 18A-18B show CD6 protein levels after itolizumab treatment. FIG. 18A shows cell associate CD6 levels and FIG. 18B shows soluble CD6 (sCD6) levels in the cell supernatant.

FIG. 19 shows that itolizumab induces cleavage of cell surface CD6 and a concomitant increase in soluble CD6.

FIGS. 20A-20B illustrate the role of monocytes in itolizumab-induced cleavage of CD6 from the cell surface of T-lymphocytes.

FIGS. 21A-21E that itolizumab-induced decrease in cell surface levels of CD6 correlates with decreased T cell activation.

FIG. 22 shows that that CD6^{low} cells generated by initial incubation with itolizumab not only remained CD6^{low} but were also less T_{eff}-like (e.g., less Tₕ1 and Tₕ17-like) following subsequent stimulation with CD3+ALCAM.

FIGS. 23A-23D show that itolizumab reduces the alloreactivity of T lymphocytes.

FIGS. 24A-24B show the effects of initial itolizumab treatment on the subsequent generation of T_{reg} lymphocytes. FIG. 24A shows naïve CD4+ T cell isolation and CD6 loss in naïve CD4+ T cells (CD3+CD4+CD45RA+CD45RO−) isolated from PBMCs previously treated with isotype or itolizumab to generate CD6^{high} or CD6^{low} naïve T cells, respectively. FIG. 24B shows the generation of T_{regs} following pretreatment with isotype control (CD6^{high}) or itolizumab (CD6^{low}), as indicated by positive staining with FoxP3 and Helios.

FIG. 25 shows that CD6^{low} T_{regs} have increased suppressive activity relative to CD6^{high} T_{regs}.

FIG. 26A shows that itolizumab-induced CD6 cell surface loss occurs in a dose-dependent manner (CD6 detection on PBMCs from two donors after 24 hour incubation with a concentration range of itolizumab, as indicated). FIG. 26B shows that the assay is able to distinguish changes in glycosylation state.

FIGS. 27A-27E show loss of cell surface CD6 in T cells from SLE patients treated with itolizumab.

FIGS. 28A-28B show that cell surface CD6 on CD4 (28A) and CD8 (28B) T cells decreases following the first dose of itolizumab in SLE patients, and that a greater loss of surface CD6 is observed with higher doses of itolizumab.

FIGS. 29A-29B show that treatment with itolizumab at 0.8 mg/kg caused a 3-fold reduction in Tₕ17 cells and a 2.5-fold increase in T_{reg} cells as compared to placebo.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

Certain embodiments include methods of identifying an optimized anti-CD6 antibody. In some embodiments, the methods of identifying an optimized anti-CD6 antibody are based on measurements of selectivity, including measurements of avidity. In some embodiments, the methods of identifying an optimized anti-CD6 antibody comprises measuring loss of cell surface CD6 over time after treatment. In some embodiments, the optimized anti-CD6 antibody is a fab, f(ab)₂, or fusion protein.

Some embodiments include methods for selectively binding to cells with high levels of cell surface CD6, or CD6^{high} cells. In some embodiments, the methods comprise removing or otherwise reducing CD6 from the cell surface of CD6^{high} cells. In some embodiments, the CD6^{high} cells are T-lymphocytes. In some embodiments, the T-lymphocytes are one or more of CD4 T helper (Th1, Th2, Th22, Th9, Th17, Tfh, Tph), CD8 naïve, effector, effector memory, stem memory or central memory CD4 or CD8 cells, natural killer (NK) cells, and innate lymphoid cells (ILC), for example, ILC1, ILC2, and/or ILC3 cells.

Some embodiments provide methods for selectively removing or reducing cell surface CD6 from CD6^{high} cells. In some embodiments, the methods comprise treatment with an optimized anti-CD6 antibody. In some embodiments, CD6 surface expression is measured. Some embodiments provide methods of determining therapeutic dosages of an optimized anti-CD6 antibody comprising measuring loss of cell surface CD6 over time after treatment. In some embodiments, the antibody is itolizumab. Some embodiments provide methods of decreasing a cell's pathogenicity, comprising administering an optimized anti-CD6 antibody. Some embodiments include methods of screening an anti-CD6 antibody, or antigen binding fragment thereof, for use as a biological therapeutic comprising: incubating the candidate anti-CD6 antibody, or antigen binding fragment thereof, with cells that express CD6 on the cell surface; measuring cell surface CD6 expression on the cells; and formulating the candidate anti-CD6 antibody, or antigen binding fragment thereof, as a pharmaceutical composition if it decreases cell surface CD6 expression relative to a control or standard.

Certain embodiments include methods of selectively decreasing cell surface CD6 levels on T-lymphocytes, and selectively decreasing levels of CD6^{high} T-lymphocytes and/or T_{eff} cells in a subject. As used herein, “T_{eff} cells” include activated T helper cells such as Th1 cells and Th17 cells. In specific embodiments, the “T_{eff} cells” are Th17 cells.

Certain embodiments include obtaining a baseline cell surface CD6 measurement and/or defining a threshold or target level of cell surface CD6, and administering a therapeutic dose of an anti-CD6 antibody such as itolizumab. In some embodiments, the methods comprise monitoring CD6 levels in a tissue sample from the subject, wherein the tissue sample comprises T-lymphocytes. In some embodiments, the methods comprise administering an additional dose of itolizumab to the subject if the cell surface CD6 levels return to about or more than about 25, 30, 35, 40, 45, 50, 55, 60, 65, 70, or 75 percent of the baseline cell surface CD6 measurement. Certain embodiments include administering an additional dose of itolizumab if the cell surface CD6 levels rise to about or above the target level of cell surface CD6 (see, e.g., FIGS. 17A-17B).

Certain embodiments provide methods of preventing or ameliorating GVHD in a patient in need of transplantation comprising incubating the tissues to be transplanted with an anti-CD6 antibody that selectively strips, removes, or otherwise reduces cell surface CD6 from CD6^{high} cells; and transplanting the tissues into the patient. In some embodiments, the methods comprises measuring patient CD6 levels. In some embodiments, the methods comprise a baseline measurement.

Some embodiments provide methods of modulating the ratio of T_{eff} cells to T_{reg} cells. Also disclosed herein are methods for increasing the ratio of T_{reg} cells to T_{eff} cells.

Also included are methods of converting a T_{eff} cell to a T_{reg} cell comprising treatment with an optimized anti-CD6 antibody. In certain embodiments, the optimized anti-CD6 antibody is itolizumab.

Certain embodiments include methods of selectively targeting T_{eff} cells. Some embodiments provide methods of selectively targeting T_{eff} cells comprising administering an optimized anti-CD6 antibody. In some embodiments, the anti-CD6 antibody is itolizumab.

Some embodiments provide methods of selectively removing CD6 from pathogenic T cells. In some embodiments, the methods comprise administering an optimized anti-CD6 antibody. In some embodiments, the antibody is itolizumab. Certain embodiments provide methods of selectively attenuating pathogenic T cells. In certain embodiments, the methods comprise administering an optimized anti-CD6 antibody. In some embodiments, the antibody is itolizumab.

Certain embodiments relate to methods of generating hyporesponsive T cells. In certain embodiments, the methods comprise administering an optimized anti-CD6 antibody. In some embodiments, the antibody is itolizumab. Some embodiments provide methods of reducing auto-reactivity in T cells. Some embodiments provide methods of reducing alloreactivity in T cells.

Disclosed herein are methods of selectively targeting CD6^{high} cells. In some embodiments, the selectively targeted CD6 cells comprise subsets of CD4 T helper (Th1, Th2, Th22, Th9, Th17, Tfh, Tph) and CD8 cells of naive, effector, effector memory, stem memory and central memory subtypes; natural killer T cells and innate lymphoid cells. Also disclosed herein are methods for modulating the ratio of T_{eff} cells to T_{reg} cells. Also disclosed herein are methods for sparing T_{reg} cells. Also disclosed herein are methods for converting T_{eff} cells to T_{reg} cells. Also disclosed herein are methods of using an optimized anti-CD6 antibody to modify T cell responses and to attenuate disease.

In some embodiments, the present disclosure provides methods of engaging CD6 with an anti-CD6 antibody such that the anti-CD6 antibody only binds to cells with high levels of expression of cell surface CD6 and wherein the antibody induces a sustained loss of cell surface CD6. Other aspects of the present disclosure are described herein.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references. For the purposes of the present disclosure, the following terms are defined below.

The articles “a” and “an” are used herein to refer to one or to more than one (i.e., to at least one) of the grammatical object of the article. By way of example, “an element” means one element or more than one element.

The term “and/or” is used in this disclosure to mean either “and” or “or” unless indicated otherwise.

The term “e.g.” is used herein to mean “for example,” and will be understood to imply the inclusion of a stated step or element or group of steps or elements but not the exclusion of any other step or element or group of steps or elements.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight, or length.

The term “administering”, as used herein, refers to any mode of transferring, delivering, introducing, or transporting matter such as a compound, e.g. a pharmaceutical compound, or other agent such as an antigen, to a subject. Modes of administration include oral administration, topical contact, intravenous, intraperitoneal, intramuscular, intranasal, or subcutaneous administration. Administration “in combination with” further matter such as one or more therapeutic agents includes simultaneous (concurrent) and consecutive administration in any order.

The term “avidity”, as used herein, refers to the measure of the strength of binding between an antigen-binding molecule (such as an anti-CD6 antibody) and the pertinent antigen. Avidity is related to both the affinity between an antigenic determinant and its antigen binding site on the antigen-binding molecule and the number of pertinent binding sites present on the antigen-binding molecule.

The term “binding partner” as used herein refers to matter, such as a molecule, in particular a polymeric molecule, that can bind a nucleic acid molecule such as a DNA or an RNA molecule, including an mRNA molecule, as well as a peptide, a protein, a saccharide, a polysaccharide or a lipid through an interaction that is sufficient to permit the agent to form a complex with the nucleic acid molecule, peptide, protein or saccharide, a polysaccharide or a lipid, generally via non-covalent bonding. In some embodiments, the binding partner is an immunoglobulin or a proteinaceous binding molecule with immunoglobulin-like functions as defined below. In some embodiments, the binding partner is an aptamer. In some embodiments, a binding partner is specific for a particular target. In some embodiments, a binding partner includes a plurality of binding sites, each binding site being specific for a particular target. As an illustrative example, a binding partner may be a proteinaceous agent with immunoglobulin-like functions with two binding sites. It may for instance be antigen binding fragment of an antibody. It may for instance be a bispecific diabody, such as a bispecific single chain diabody.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

As used herein, the term “chimeric antibody” refers to an immunoglobulin polypeptide or domain antibody that includes sequences from more than one species. In a chimeric antibody a heavy chain or a light chain may contain a variable region sequence from one species such as human and a constant region sequence from another species such as mouse. As an example, a “chimeric antibody” may be an immunoglobulin that has variable regions derived from an animal antibody, such as a rat or mouse antibody, fused to another molecule, for example, the constant domains derived from a human antibody. The term “chimeric antibody” is intended to encompass antibodies in which: (i) the heavy chain is chimeric but the light chain comprises Y and C regions from only one species; (ii) the light chain is chimeric but the heavy chain comprises Y and C regions from only one species; and (iii) both the heavy chain and the light chain are chimeric.

An “effective amount,” when used in connection with a compound, is an amount of the compound, such as an anti-CD6 antibody (e.g., itolizumab or EQ001), needed to elicit a desired response. In some embodiments, the desired response is a biological response, e.g., in a subject. In some embodiments, the compound (e.g., an anti-CD6 antibody) may be administered to a subject in an effective amount to effect a biological response in the subject. In some embodiments, the effective amount is a “therapeutically effective amount.”

The terms “therapeutically effective amount” and “therapeutic dose” are used interchangeably herein to refer to an amount of a compound, such as an anti-CD6 antibody (e.g., itolizumab or EQ001), which is effective following administration to a subject for treating a disease or disorder in the subject as described herein.

The term “pathogenicity” is used herein to refer to a T cell's ability to exhibit a pathogenic response in terms of increased proliferation and secretion of cytokines.

The term “prophylactically effective amount” is used herein to refer to an amount of a compound, such as an anti-CD6 antibody (e.g., itolizumab or EQ001), which is effective following administration to a subject, for preventing or delaying the onset of a disease or disorder in the subject as described herein.

In this regard, a “humanized antibody” as used herein is an immunoglobulin polypeptide or domain antibody containing structural elements of a human antibody and the antigen binding site of a non-human antibody. “Humanized antibodies” contain a minimal number of residues from the non-human antibody from which they are derived. For instance, they may contain only the CDR regions of the non-human antibody, or only those residues that make up the hypervariable regions of the non-human antibody. They may also contain certain residues from outside the variable regions of the non-human polypeptide, such as residues that are necessary to mimic the structure of the non-human antibody or to minimize steric interference. Typically a humanized antibody contains a human framework, at least one CDR from a non-human antibody, with any constant region present being substantially identical to a human immunoglobulin constant region, i.e., at least about 85-90%, such as at least 95% identical. Hence, in some instances all parts of a humanized immunoglobulin, except possibly the CDRs, are substantially identical to corresponding parts of one or more native human immunoglobulin sequences. In addition, humanized antibodies may contain residues that do not correspond to either the human or the non-human antibodies.

As used herein, the term “antibody fragment” refers to any form of an antibody other than the full-length form. Antibody fragments herein include antibodies that are smaller components that exist within full-length antibodies, and antibodies that have been engineered. Antibody fragments include, but are not limited to, Fv, Fc, Fab, and (Fab′)2, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, and bispecific antibodies. Unless specifically noted otherwise, statements and claims that use the term “antibody” or “antibodies” may specifically include “antibody fragment” and “antibody fragments.”

The term “VH” is used herein to denote the variable heavy chain of an antibody.

The term “VL” is used herein to denote the variable light chain of an antibody.

The term “antigen binding fragment” in reference to an antibody refers to any antibody fragment that retains binding affinity for an antigen to which the parent full length antibody binds, and antigen binding fragments include, but are not limited to, Fv, Fab, (Fab′)2, scFv, diabodies, triabodies, tetrabodies, bifunctional hybrid antibodies, CDR1, CDR2, CDR3, combinations of CDRs, variable regions, heavy chains, light chains, and bispecific antibodies.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “modulating” includes “increasing,” “enhancing” or “stimulating,” as well as “decreasing” or “reducing,” typically in a statistically significant or a physiologically significant amount as compared to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the amount produced by no composition (e.g., in the absence of any of the anti-CD6 antibodies of the disclosure) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally-occurring amino acids, such as a chemical analogue of a corresponding naturally-occurring amino acid, as well as to naturally-occurring amino acid polymers.

A “subject,” or “patient” as used herein, includes any animal that exhibits a symptom, or is at risk for exhibiting a symptom, which can be treated or diagnosed with an anti-CD6 antibody, or an antigen binding fragment thereof. Suitable subjects (patients) includes, preferably, human patients. Suitable subjects also include laboratory animals (such as mouse, rat, rabbit, or guinea pig), farm animals (such as pig, horse, cow), and domestic animals or pets (such as a cat or dog). Non-human primates (such as a monkey, chimpanzee, baboon or rhesus) are also included.

“Substantially” or “essentially” means nearly totally or completely, for instance, 95% or greater of some given quantity.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

The present disclosure relates, for example, to methods of identifying anti-CD6 antibodies capable of removing cell surface CD6 from CD6^{high} cells and methods of use of anti-CD6 antibodies to convert T_{eff} to T_{reg|} cells and methods of treatment, prevention, or attenuation of auto or allo-reactive T cells, natural killer (NK) cells, or innate lymphoid cells (ILC) comprising administering an anti-CD6 antibody to a subject.

CD6 is an important cell surface protein predominantly expressed by human NK cells, ILC, T cells and a subset of B cells, as well as by some B cell chronic lymphocytic leukemias and neurons [Aruffo et ah, J. Exp. Med. 1991, 174:949; Kantoun et ah, J. Immunol. 1981, 127:987; Mayer et al., J. Neuroimmunol. 1990. 29: 193] CD6 is a member of a large family of proteins characterized by having at least one domain homologous to the scavenger receptor cysteine-rich domain (SRCR) of type I macrophages [Matsumoto, et al., J. Exp. Med. 1991, 173:55 and Resnick et al., Trends Biochem. Sci. 1994, 19:5] Other members of this family include CD5 [Jones et al., Nature. 1986, 323 :346]; cyclophilin C [Friedman et al. 1993, PNAS 90:6815]; complement factor I, which binds activated complement proteins C3b and C4b [Goldberger, et al., J. Biol. Chem. 1987, 262: 10065]; bovine WC-1 expressed by .tau./.delta. T cells [Wijingaard et al., J. Immunol. 1992, 149:3273] and M130 [Law et al., Eur J. Immunol. 1993, 23:2320], a macrophage activation marker.

The extracellular domain of the mature CD6 protein is composed of three SRCR domains (hereinafter designated D1, D2, and D3). D3 corresponding to the membrane proximal SRCR domain followed by a short 33-amino-acid stalk region. These extracellular domains are anchored to the cell membrane via a short transmembrane domain followed by a cytoplasmic domain of variable length [Aruffo et al., J. Exp. Med. 1991, 174:949].

A soluble form of CD6 (sCD6) of unknown origin has been reported to circulate at very low levels (pico/nano molar range) in sera from healthy individuals has been reported. Elevated levels of sCD6 were observed in individuals with systemic inflammatory response syndrome and primary Sjogren's syndrome, but direct mechanistic and functional relationships between these events are lacking. Reports suggest that sCD6 is formed by shedding of the membrane bound receptor via the proteolytic action of members of the ADAM family of metalloproteinases (Carrasco, et al., Frontiers in Immunology 2017. 8:769). Further, although the functional role of sCD6 in T-cell physiology is not yet known, in vitro results suggest that sCD6 inhibits T cell activation and maturation of the immunological synapse prompting some investigators to posit that sCD6 acts as a decoy receptor to inactivate bystander T cells near a site of inflammation.

Studies using CD6-immunoglobulin fusion proteins, containing selected extracellular domains of CD6 fused to human IgG1 constant domains (CD6-Rgs), led to the identification and cloning of a CD6 ligand, designated “activated leukocyte cell adhesion molecule” (ALCAM) also known as CD166 [Patel, et al., J. Exp. Med. 1995. 181: 1563-1568; Bowen et al., J. Exp. Med 1995, 181:2213-2220]

ALCAM is a 100-105 kD type I transmembrane glycoprotein that is a member of the immunoglobulin superfamily and comprises five extracellular immunoglobulin domains (2 NH2-terminal, membrane-distal variable-(V)-type (VI, V2 or D1, D2) and 3 membrane-proximal constant-(C2)-type Ig folds) [C1, C2, C3], a transmembrane region, and a short cytoplasmic tail. The N-terminal domain (D1) is exclusively involved in ligand binding, whereas membrane proximal domains (C2, C3 or D4, D5) are required for homophilic interactions.

ALCAM binds to domain 3 of CD6 corresponding to the membrane proximal SRCR domain [Whitney, et. al., J. Biol. Chem 1995, 270: 18187-18190].

Studies of the role of CD6/ALCAM interactions in T-cell regulation have shown that this receptor-ligand pair is able to mediate the adhesion of CD6 expressing cells to thymic epithelial cells. This suggests that CD6/ALCAM interactions are important for modulating T-cell development and activation. Moreover, ALCAM shedding has also been reported, and, like with sCD6, the process appears to be the product of ADAM family metalloproteinase-mediated cleavage.

CD6 plays an important role in modulating T-cell function in vivo. CD6 is also reported to be part of the immunologic synapse mediating early and late T-cell-antigen presenting cells (APC) interaction. Moreover, it has been shown that CD6^{high} T cells are pathogenic are CD6^{low} T cells are not pathogenic (see, for example, Ma et al., Journal of Crohn's and Colitis. 13: 510-524, 2019).). Methods for establishing high vs. low CD6 expression are known in the art, and CD6^{high} and CD6^{low} cells can be defined by comparing protein and/or mRNA expression on various cell subsets (see, for example, Ma et al., supra; and Santana et al., Cytometry A. 85:901-8, 2014).

U.S. Pat. No. 6,372,215 discloses antibodies and other binding agents that bind specifically to SRCR domains 3 (D3) of human CD6 (hCD6) or human CD6 stalk domain (CD6S) and inhibit activated leukocyte cell adhesion molecule (ALCAM) binding to CD6.

Earlier publications and patents disclosed sequences of the murine anti-CD6 (TOR-TO monoclonal antibody and the amino acid modifications that were carried out to humanize IOR-T1 to T1h (humanized IOR-T1). U.S. Pat. No. 5,712,120 and its equivalent EP 0699755 disclose specific methods to humanize murine monoclonal antibodies and the sequence of IOR-T1 and T1h. U.S. Pat. No. 6,572,857 and its equivalent EP 0807125 disclose the sequence of IOR-T1 and T1h (humanized IOR-T1). PCT/IN2008/00562, entitled “A Monoclonal Antibody and a Method Thereof,” discloses the production of an anti-CD6 antibody in NS0 cells, which has the heavy and light chain sequences provided herein as SEQ ID NOS: 1 and 2. The INN name for this antibody is itolizumab. Itolizumab is produced in the mouse derived NS0 cell line and in Chinese Hamster Ovary (CHO) cells, and is referred to herein by its trade name EQ001, when produced in CHO cells and by its trade name ALZUMAB, when produced in NS0 cells. EQ001 (i.e., itolizumab produced in CHO cells) is also known in the art as “Bmab-600.” Certain embodiments refer to the antibody itself, irrespective of its production method, by its INN name, itolizumab. Thus, the term itolizumab, as used herein, encompasses ALZUMAB and EQ001, each of which have the same sequence as itolizumab (see Table S1). The amino acid sequences of the variable heavy (VH) and variable light (VL) of itolizumab (and EQ001/ALZUMAB) are provided herein as SEQ ID NOS: 1 and 2, respectively. The nucleotide (DNA) sequences of the VH and VL of itolizumab (and EQ001/ALZUMAB) are provided herein as SEQ ID NOS: 3 and 4, respectively. The amino acid sequence of the itolizumab (and EQ001/ALZUMAB) VH CDRs 1-3 are provided as SEQ ID NOS: 5-7, respectively. The amino acid sequence of the itolizumab (and EQ001/ALZUMAB) VL CDRs 1-3 are provided as SEQ ID NOS: 8-10, respectively.

Antibodies targeting CD6 have shown promise as therapies for a wide-range of diseases and conditions that are caused, at least in part, by aberrant T cell activity. For example, PCT/IN2008/000562 discloses the use of itolizumab to inhibit the proliferation of naive T cells and to treat various inflammatory disorders including multiple sclerosis, transplant rejection, rheumatoid arthritis, and psoriasis. Indeed ALZUMAB is currently marketed in India for the treatment of psoriasis. Further, the use of itolizumab to treat lupus is disclosed in PCT/IB2017/056428. However, due to the heterogeneity of these diseases and their tendency to cycle between difference disease forms mediated by T cells, B cells, dendritic cells, monocytes, and neutrophils, more targeted treatment therapies are needed to more fully tap the potential of these antibodies.

As of yet, no biomarker strategy is employed clinically to determine when a patient might be most likely to respond favorably to treatment with an anti-CD6 antibody (e.g., itolizumab) or more generally to a reduction of cell surface CD6 expression on CD6^{high} cells. Embodiments of the present disclosure include methods of identifying dosing regimens to obtain the desired attenuation of CD6^{high} cells.

Certain embodiments include methods for determining an optimal dosage of itolizumab in a human subject having an autoimmune, immuno-inflammatory, or inflammatory disease, graft versus host disease (GVHD), or organ transplant rejection, comprising:
(a) determining a baseline of cell surface CD6 levels in a tissue sample from the subject, wherein the tissue sample comprises T-lymphocytes, and optionally defining a target level of cell surface CD6 in the subject;
(b) administering to the subject a series of two or three or more dosages of itolizumab (for example, 3, 4, 5, or 6 dosages), optionally increasing dosages (e.g., up to about 4 mg/kg), for example, increasing with each other dosage or every other dosage (e.g., increasing by about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, or 1.0 mg/kg or more per dose or per every other dose);
(c) monitoring cell surface CD6 levels in a tissue sample from the subject between the series of dosages, wherein the tissue sample comprises T-lymphocytes;
(d) identifying the lowest dosage from the series of dosages as being the optimal dosage if cell surface CD6 levels in step (c) are about or less than about 5, 10, 15, 20, 25, 30, 40, 45, or 50 percent of the baseline from (a), or are within about or less than about 5, 10, 15, or 20 percent of the optional target level from (a), and if no further reductions (e.g., statistically significant reductions) in cell surface CD6 levels are observed between the series of dosages. Certain embodiments include determining cell surface CD6 levels on CD4 cells and/or CD8 cells in the tissue sample from the subject. In particular embodiments, the tissue sample is a blood sample. Some embodiments include defining the target level of cell surface CD6 in the subject based on clinical parameters or symptoms of the disease.

Some embodiments relate to a dosing regimen for treatment of an autoimmune, immuno-inflammatory, or inflammatory disease, graft versus host disease (GVHD), or organ transplant rejection in a human subject in need thereof, comprising:
(a) determining a baseline of cell surface CD6 levels in a tissue sample from the subject, wherein the tissue sample comprises T-lymphocytes, and optionally defining a target level of cell surface CD6 in the subject;
(b) administering to the subject a dosage of itolizumab, which reduces cell surface CD6 levels in T-lymphocytes in the subject to about or less than about 5, 10, 15, 20, 25 percent of the baseline from (a);
(c) monitoring cell surface CD6 levels in a tissue sample from the subject, where the tissue sample comprises T-lymphocytes; and
(d) administering a further dosage of itolizumab to the subject before or if the cell surface CD6 levels in (c) return to about or more than about 25, 30, 35, 40, 45, 50, 55, 60, 65, 70, or 75 percent of the baseline from (a), or rise to about or above the target level from (a).

In some embodiments, the dosing regimen maintains cell surface CD6 levels in T-lymphocytes (e.g., CD4 and/or CD8 cells) from the subject at about or lower than about 40, 45, 50, 55, 60, 65, 70, or 75 percent of the baseline from (a), or within about 5, 10, 15, or 20 percent of the optional target level from (a), and certain embodiments include defining the target level of cell surface CD6 in the subject based on clinical parameters or symptoms of the disease.

Also included are dosing regimens for treatment of an autoimmune, immuno-inflammatory, or inflammatory disease, graft versus host disease (GVHD), or organ transplant rejection in a human subject in need thereof, comprising:
(a) determining a baseline level of CD6^{high} T-lymphocytes in a blood sample from the subject, and optionally defining a target level of CD6^{high} T-lymphocytes;
(b) administering to the subject a dosage of itolizumab, which reduces the level of CD6^{high} T-lymphocytes in the subject to about or less than about 5, 10, 15, 20, 25, 30, or 40 percent of the baseline from (a);
(c) monitoring levels of CD6^{high} T-lymphocytes in a blood sample from the subject; and
(d) administering a further dosage of itolizumab to the subject before the levels of CD6^{high} T-lymphocytes from (c) return to about or more than about 25, 30, 35, 40, 45, 50, 55, 60, 65, 70, or 75 percent of the baseline level from (a), or rise to about or above the target level from (a), and in some instances, defining the target level of CD6^{high} T-lymphocytes in the subject based on clinical parameters or symptoms of the disease.

In some embodiments, the dosing regimen maintains levels of CD6^{high} T-lymphocytes in the subject at about or less than about 45, 50, 55, 60, 65, 70, or 75 percent of the baseline level from (a), including wherein the T-lymphocytes are CD4 cells and/or CD8 cells. In some embodiments, the dosing regimen decreases the ratio of CD6^{high}:CD6^{low} T-lymphocytes in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally wherein the T-lymphocytes are CD4 cells and/or CD8 cells.

In some embodiments, the dosing regimen decreases the ratio of T_{eff}:T_{reg} cells in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally CD4 cells and/or CD8 cells.

Also included are cell-based therapeutic methods of preventing or ameliorating graft versus host disease (GVHD) in a human transplant patient comprising:
(a) incubating a transplant tissue with an anti-CD6 antibody, optionally itolizumab, for a time sufficient to reduce cell surface levels of CD6 on CD6^{high} T-lymphocytes in the transplant tissue; and
(b) transplanting the transplant tissue into the patient.

Certain methods include determining cell surface levels of CD6 on T-lymphocytes in the transplant tissue before and after step (a), and performing step (b) if cell surface levels of CD6 after step (a) are reduced by about or at least about 10, 20, 30, 40, 50, 60, 70, 80, 90, 100, 200, 300, 400, 500 percent or more relative to before step (a). In some embodiments, the transplant tissue comprises umbilical cord blood cells, bone marrow cells, peripheral blood cells, mobilized peripheral blood cells, mesenchymal stem cells, hematopoietic stem cells, cells differentiated from stem cells/progenitor cells, engineered cells (for example, chimeric antigen receptor (CAR) cells such as CAR T cells), or any combinations of said cells. In some embodiments, the transplant tissue is autologous to the human transplant patient, that is, wherein the transplant tissue is removed from the patient, treated as described herein, and later administered to that same patient. In some embodiments, the transplant tissue is an allotransplant tissue, or a tissue that is allogeneic to the human transplant patient, for instance, wherein the transplant tissue is obtained from a genetically non-identical human donor, treated as described herein, and then administered to the human patient.

Also included are cell-based therapeutic methods (for example, T_{reg} therapies) for treatment or amelioration of an autoimmune, immuno-inflammatory, or inflammatory disease in a human patient in need thereof, comprising:
(a) incubating a transplant tissue with an anti-CD6 antibody, optionally itolizumab, for a time sufficient to reduce cell surface levels of CD6 on CD6^{high} T-lymphocytes in the transplant tissue, thereby generating transplant tissue enriched for CD6^{low} (naïve) T-lymphocytes;
(b) treating the transplant tissue from (a) for a time sufficient to generate T_{reg} lymphocytes from the CD6^{low} (naïve) T-lymphocytes; and
(c) transplanting the transplant tissue from (c) into the patient.

Similar to above, certain embodiments comprise the step(s) of determining cell surface levels of CD6 on T-lymphocytes in the transplant tissue before and after step (a), and performing step (b) if cell surface levels of CD6 after step (a) are reduced by about or at least about 10, 20, 30, 40, 50, 60, 70, 80, 90, 100, 200, 300, 400, 500 percent or more relative to before step (a).

In some embodiments, step (b) above comprises incubating the transplant tissue enriched for CD6^{low} (naïve) T-lymphocytes with a combination of T cell activators/stimulatory signals, cytokines, growth factors, transcription factors, and/or stabilization agents (for example, CD3/CD28 activators, T_{reg} differentiation cocktails such as IMMUNOCULT™ Human T_{reg} Differentiation Supplement by STEMCELL TECHNOLOGIES™) for a time sufficient to generate or enrich for T_{reg} lymphocytes. Examples of such treatments or protocols for generating (e.g., differentiating, expanding) T_{regs} are known in the art (see, for example, Golovina et al., PLoS ONE (2011) 6:e15868. doi: 10.1371/journal.pone.0015868; Scotty et al., Haematologica 98:1291-9, 2013; Dons et al., Hum Immunol. 73:328-34, 2012; Fraser et al., Mol Ther Methods Clin Dev. 8:198-209, 2018; Romano et al., Front. Immunol. 31 Jan. 2019, doi.org/10.3389/fimmu.2019.00043; Haddadi et al., Clinical and Experimental Immunol. 201(2):205-221, 2020), including a protocol that combines RORγ-specific inverse agonist (SR) and a TGF-β signaling promoter [N-acetyl puromycin (N-Ac)]).

Additional protocols for generating T_{reg} lymphocytes are described, for example, in Chen et al. (The Journal of Experimental Med. 198(12):1875-1886, 2003), which describes costimulation of naïve T cells with TCRs and TGF-β; Zheng et al. (J. Immunol. 178:2018-2027, 2007), which describes stimulation with IL-2 and TGF-β; Schiavon et al. (PNAS. 116:6298-6307, 2019), which describes stimulation with IL-2, TGF-β, and PGE₂; and Ferreira et al. (Nat Rev Drug Discov. 18(10): 749-769, 2019), which describes a variety of techniques and clinical trials that illustrate the ex vivo generation of T_{reg} lymphocytes. Zagury et al. (WO 2018/024896) describe exemplary protocols including culturing T cells in the presence of a γδ T cell activator and the following agents: i) an cAMP (Cyclic adenosine monophosphate) activator, ii) a TGFP (Transforming growth factor beta) pathway activator, iii) a mTOR inhibitor, optionally iv) at least one cytokine selected in the group of IL-2, IL-7, IL-15 and TSLP, and optionally v) at least one TET enzymes activator and/or at least one DNMT inhibitor. Alvarez-Salazer et al. (Front. Immunol. 11:375. doi: 10.3389/fimmu.2020.00375) describe the large-scale generation of human T_{reg} lymphocytes with functional stability for use in immunotherapy in transplantation, for example, by co-culturing naïve T cells with dendritic cells in the presence of TGF-β1, IL-2, and retinoic acid to generate T_{regs}, and expanding the T_{regs} in the presence of TGF-β1, IL-2, and rapamycin. The methods described herein (e.g., step (b) above) can employ any one or more the foregoing methods, or others in the art, to generate T_{regs} from an itolizumab-enriched population of CD6^{low} (naïve) T-lymphocytes.

In some embodiments, the transplant tissue comprises umbilical cord blood cells, bone marrow cells, peripheral blood cells, mobilized peripheral blood cells, mesenchymal stem cells, hematopoietic stem cells, cells differentiated from stem cells/progenitor cells, engineered cells (for example, chimeric antigen receptor (CAR) cells such as CAR T cells), or any combinations of said cells. In some embodiments, the transplant tissue is autologous to the human patient, for example, an autologous T_{reg} therapy. In certain embodiments, transplant tissue is an allotransplant tissue, or a tissue that is allogeneic to the human patient.

Certain embodiments include methods for treatment of an autoimmune, immuno-inflammatory, or inflammatory disease, graft versus host disease (GVHD), or organ transplant rejection in a human subject in need thereof, comprising:
(a) administering itolizumab to the subject; and
(b) determining levels of cell surface CD6 on T-lymphocytes in a tissue sample from the subject, determining levels of CD6^{high} and optionally CD6^{low} T-lymphocytes in a tissue sample from the subject, and/or determining levels/ratios of T_{eff} and T_{reg} cells in the subject, wherein the tissue sample comprises T-lymphocytes;
wherein the administration of itolizumab reduces any one or more of (i) levels of cell surface CD6 on T-lymphocytes, optionally CD4 and/or CD8 cells; (ii) levels of CD6^{high} T-lymphocytes in the subject; (iii) the ratio of CD6^{high}:CD6^{low} T-lymphocytes in the subject; and/or (iv) the ratio of T_{eff}:T_{reg} cells in the subject, and thereby reduces a pathogenic immune response in the subject. Methods for determining any one or more of (i)-(iv) above are described herein (see the Examples) and known in the art.

In some instances, the administration of itolizumab decreases cell surface CD6 levels on T-lymphocytes in the subject by about or at least about 10, 20, 30, 40, 50, 60, 70, 80, 90, 100, 200, 300, 400, 500 percent or more relative to a control or standard or baseline, optionally wherein the T-lymphocytes are CD4 cells and/or CD8 cells; decreases levels of CD6^{high} T-lymphocytes in the subject by about or at least about 10, 20, 30, 40, 50, 60, 70, 80, 90, 100, 200, 300, 400, 500 percent or more relative to a control or standard or baseline, optionally wherein the T-lymphocytes are CD4 cells and/or CD8 cells; decreases the ratio of CD6^{high}:CD6^{low} T-lymphocytes in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally wherein the T-lymphocytes are CD4 cells and/or CD8 cells; and/or decreases the ratio of T_{eff}:T_{reg} cells in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline.

In some embodiments, the autoimmune, immuno-inflammatory, or inflammatory disease in the patient or subject is characterized by an increased ratio of T_{eff}:T_{reg} cells relative to a standard or healthy subject. In specific embodiments, the ratio of T_{eff}:T_{reg} cells in the subject or patient is increased by about or at least about 1.5, 2, 3, 4, 5, 6, 7, 8, 9, or 10-fold or more relative to the standard or healthy subject. In specific embodiments, the autoimmune, immuno-inflammatory, or inflammatory disease is inflammatory bowel disease (IBD), optionally Crohn's disease or ulcerative colitis, systemic lupus erythematosus (SLE), optionally SLE with lupus nephritis, rheumatoid arthritis (RA), multiple sclerosis (MS), psoriasis, psoriatic arthritis, ankyolosing spondylitis, or asthma.

Certain embodiments include in vitro cell-based (that is, cell culture-based) methods for analyzing a test lot of itolizumab, comprising
(a) incubating the test lot of itolizumab with (cultured) cells that express CD6 on the cell surface;
(b) measuring cell surface CD6 expression on the cells; and
(c) formulating the test lot of itolizumab as a pharmaceutical composition if the test lot decreases cell surface CD6 expression (for example, on T-lymphocytes) relative to a control or standard, and rejecting the test lot of itolizumab if the test lot does not decrease cell surface CD6 expression relative to the control or standard.

In some embodiments, step (b) comprises directly measuring cell surface CD6 expression on the cells, for example, T-lymphocytes. Cell surface CD6 levels can be measured according to a variety of techniques in the art, for example, by flow cytometry, cytometry by time-of-flight (CyToF), cellular ELISA, or immunofluorescent microscopy. In certain embodiments, step (b) comprises indirectly measuring/determining cell surface CD6 expression by measuring soluble CD6 in the cell supernatant, which serves as in indicator or proxy for cell surface CD6 expression. In these and realted embodiments, an increase in soluble CD6 in the supernatant indicates a decrease in cell surface CD6 expression. Exemplary methods for measuring soluble CD6 in the cell supernatant include enzyme-linked immunosorbent assay (ELISA), chemiluminescence assay, electrochemiluminescence assay, high performance liquid chromatography, western blot, and immunoprecipitation followed by western blot, among others known in the art.

In some embodiments, the cells comprise peripheral blood mononuclear cell (PBMCs), which typically comprise progenitor populations such as CD14+ monocytes and lymphocytes such CD19+B cells, CD4+ helper T cells, CD8+ cytotoxic T cells, and CD56+ natural killer (NK) cells. In particular embodiments, the cells comprise a human T cell line or a cell line engineered to express CD6, for example, human CD6. Examples of cell lines include MOLT-4, MOLT-3, MOLT-16, HuT 78, HuT 102, Jurkat, Jurkat NFAT, CCRF-CEM, 12.1, MJ (G11), LOUCY, SUP-T1, HEL.92.1.7, EFO-21, RPMI-8226, HPB-ALL, HH, KE37, P12ICHIKAWA, PEER, ALLSIL, RPMI8402, CMLT1, PF382, EHEB, and DU4475 cells.

In some embodiments, the cells comprise monocytes, for example, human monocytes, such as a monocyte cell line. In some embodiments, the monocyte cell line is selected from U937, THP1, MC-1010, TUR, AML-193, and MV-4-11 cells. In some embodiments, the cells comprise a mixture of (a) human T cells (for example, a human T cell line) or other human cell line engineered to express CD6, and (b) human monocytes (for example, a human monocyte cell line). In some embodiments, the mixture of (a):(b) is at a ratio of about, at least about, or no more than about 30:1, 25:1, 20:1, 15:1, 10:1, 5:1, 4:1, 3:1, 2:1, 1:1, 1:2, 1:3, 1:4, 1:5, or 1:10.

Certain embodiments include the step of formulating the test lot of itolizumab as a pharmaceutical composition if it decreases cell surface CD6 expression by about or at least about 10, 20, 30, 40, 50, 60, 70, 80, 90, 100, 200, 300, 400, 500 percent or more relative to the control or standard and/or if it increases soluble CD6 in the cell supernatant by about or at least about 10, 20, 30, 40, 50, 60, 70, 80, 90, 100, 200, 300, 400, 500 percent or more relative to the control or standard.

More generally, certain embodiments include methods of screening an anti-CD6 antibody, or antigen binding fragment thereof, for use as a biological therapeutic comprising:
(a) incubating the candidate anti-CD6 antibody, or antigen binding fragment thereof, with (cultured) cells that express CD6 on the cell surface;
(b) measuring cell surface CD6 expression on the cells; and
(c) formulating the candidate anti-CD6 antibody, or antigen binding fragment thereof, as a pharmaceutical composition if it decreases cell surface CD6 expression relative to a control or standard. Such methods can be used to identify candidate anti-CD6 antibodies that have biological properties of clinical relevance, as described herein for itolizumab.

As above, cell surface CD6 expression levels can be measured directly or indirectly according to a variety of techniques in the art. For example, cell surface CD6 expression levels can be measured directly by flow cytometry, cytometry by time-of-flight (CyToF), cellular ELISA, or immunofluorescent microscopy. Also, cell surface CD6 expression levels can be measured or determined indirectly by measuring soluble CD6 in the cell supernatant, for example, by ELISA, chemiluminescence assay, electrochemiluminescence assay, high performance liquid chromatography, western blot, and immunoprecipitation followed by western blot, among other techniques in the art.

In some embodiments, the cells comprise PBMCs. In particular embodiments, the cells comprise a human T cell line or a cell line engineered to express CD6, for example, human CD6. Examples of cell lines include MOLT-4, MOLT-3, MOLT-16, HuT 78, HuT 102, Jurkat, Jurkat NFAT, CCRF-CEM, 12.1, MJ (G11), LOUCY, SUP-T1, HEL.92.1.7, EFO-21, RPMI-8226, HPB-ALL, HH, KE37, P12ICHIKAWA, PEER, ALLSIL, RPMI8402, CMLT1, PF382, EHEB, and DU4475 cells.

In some embodiments, the cells comprise monocytes, for example, human monocytes, such as a monocyte cell line. In some embodiments, the monocyte cell line is selected from U937, THP1, MC-1010, TUR, AML-193, and MV-4-11 cells. In some embodiments, the cells comprise a mixture of (a) human T cells (for example, a human T cell line) or other human cell line engineered to express CD6, and (b) human monocytes (for example, a human monocyte cell line). In some embodiments, the mixture of (a):(b) is at a ratio of about, at least about, or no more than about 30:1, 25:1, 20:1, 15:1, 10:1, 5:1, 4:1, 3:1, 2:1, 1:1, 1:2, 1:3, 1:4, 1:5, or 1:10, including all ranges of ratios in between (e.g., 9:1).

Particular embodiments include formulating the candidate anti-CD6 antibody, or antigen binding fragment thereof, as a pharmaceutical composition if it decreases cell surface CD6 expression by about or at least about 10, 20, 30, 40, 50, 60, 70, 80, 90, 100, 200, 300, 400, 500 percent or more relative to a control or standard and/or if it increases soluble CD6 in the cell supernatant by about or at least about 10, 20, 30, 40, 50, 60, 70, 80, 90, 100, 200, 300, 400, 500 percent or more relative to the control or standard.

In certain embodiments the present disclosure provides methods of reducing the levels of CD6 on T cells in patients, comprising administering a therapeutic dose of itolizumab either through subcutaneous or intravenous administration.

In some embodiments, therapeutically effective amounts of an anti-CD6 monoclonal antibody are administered every two weeks until a patient is determined to be recovering or discharged from the hospital. In some embodiments, the doses are 3.2 mg/kg or less. In some embodiments, patients are dosed with between 0.1 to 3.2 mg/kg. In some embodiments, a single dose is administered. In some embodiments, two doses are administered. In some embodiments, between 1-12 doses are administered. In some embodiments, two doses are administered. In some embodiments, three doses are administered. In some embodiments, four doses are administered. In some embodiments, dosing is long term. In some embodiments, the dose is 100 mg. In some embodiments, the dose is 200 mg. In some embodiments, the doses are administered biweekly. In some embodiments, the doses are administered monthly. In some embodiments, the doses are administered every other month. In some embodiments, the doses are administered every six weeks. In some embodiments, the doses are administered every eight weeks. In some embodiments, the doses are administered every ten weeks. In some embodiments, the doses are administered every twelve weeks. In some embodiments, the doses are administered every three months. In some embodiments, the doses are administered every four months. In some embodiments, the doses are administered every five months. In some embodiments, the doses are administered every six months. In some embodiments, the doses are administered more than six months apart.

An exemplary, non-limiting range for a therapeutically effective amount of the anti-CD6 monoclonal antibody used in the present disclosure is about 0.01-100 mg/kg per subject body weight, such as about 0.01-50 mg/kg, for example about 0.01-25 mg/kg. In some embodiments, the ideal weight for patient's height is used to determine dose. In some embodiments, more than one dose is given to a subject. In some embodiments, a larger initial dose is given to the patient. In some embodiments, a second dose is administered after one week. In some embodiments, a second dose is administered after two weeks. In some embodiments, the second dose is the same strength as the first dose. In some embodiments, the second dose is three-fourths or less of the initial dose. In some embodiments, the second dose is half of the initial dose. In some embodiments, a third treatment is administered. In some embodiments, therapeutically effective amounts of an anti-CD6 monoclonal antibody are administered every two weeks until a patient is determined to be recovering or discharged from the hospital. In some embodiments, the doses are either 0.8 mg/kg or 1.6 mg/kg. In some embodiments, the doses administered are 1.6 mg/kg and 0.8 mg/kg. Exemplary, non-limiting doses for a therapeutically effective amount of the anti-CD6 monoclonal antibody are about or between about 0.8 mg/kg and 1.6 mg/kg. A medical professional having ordinary skill in the art may readily determine and prescribe the effective amount of the pharmaceutical composition required. For example, a physician could start doses of the anti-CD6 monoclonal antibody at levels lower than that required in order to achieve the desired therapeutic effect and gradually increase the dosage until the desired effect is achieved.

In some embodiments, the doses administered are 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, or 4.0 mg/kg. In some embodiments, the doses administered are 0.2 mg/kg, 0.4 mg/kg, 0.8 mg/kg, 1.2 mg/kg, 1.6 mg/kg, 2.0 mg/kg, or 2.2 mg/g. In some embodiment, the itolizumab dosages are 0.4 mg/kg, 0.8 mg/kg, 1.6 mg/kg, or 3.2 mg/kg. In some embodiments, the dose administered is about 25 mg, 50 mg, 75 mg, 100 mg, 125 mg, 150 mg, 175 mg, 200 mg, 225 mg, 250 mg, 275 mg, 300 mg, 325, 350, 375, 400, 425, 450, 475, or 500 mg. Included in this application are exemplary, non-limiting doses for a therapeutically effective amount of the anti-CD6 monoclonal antibody used in the present disclosure. A medical professional having ordinary skill in the art may readily determine and prescribe the effective amount of the pharmaceutical composition required. For example, a physician could start doses of the anti-CD6 monoclonal antibody at levels lower than that required in order to achieve the desired therapeutic effect and gradually increase the dosage until the desired effect is achieved. In some embodiments, the methods include analysis of the patient's blood to determine frequency of dosing. In some embodiments, patient samples are analyzed for cell surface CD6 expression. In some embodiments, patient serum is analyzed for soluble CD6.

In some embodiments, the anti-CD6 monoclonal antibody is administered by infusion in a weekly dosage of from 0.1 to 50 mg/kg per subject body weight, such as, from 0.5 to 3 mg/kg. Such administration may be repeated, e.g., 1 to 8 times, such as 2 to 4 times, or 3 to 5 times. In some instances, the administration may be performed by continuous infusion over a period of from 2 to 24 hours, such as, from 2 to 12 hours.

In some embodiments, the anti-CD6 monoclonal antibody is administered in a weekly dosage. In some embodiments, the anti-CD6 monoclonal antibody is administered in a biweekly dosage. In some embodiments, the anti-CD6 monoclonal antibody is administered in an intermittent weekly dosage. In certain embodiments, the anti-CD6 monoclonal antibody is administered in an intermittent biweekly dosage. In some of these and related embodiments, the dosage of from about 50 mg to about 350 mg of itolizumab is administered up to 7 times, such as from 2 to 4 times. In some embodiments, the anti-CD6 antibody is administered biweekly. In some embodiments, the anti-CD6 antibody is administered intermittent biweekly. In some embodiments, the intermittent biweekly dosing is a first dose followed by biweekly assessments to determine whether subsequent doses are necessary. The administration may be performed by continuous infusion over a period of about 1 hour, or over a period of about 1 to 24 hours, about 1 to 12 hours, about 1 to 6 hours, about 1 to 2 hours, or about 1, 2, 3, 4, 5, 6, 7, 8, 9, 10, 11, 12, 13, 14, 15, 16, 17, 18, 19, 20, 21, 22, 23, or 24 hours. Such regimen may be repeated one or more times as necessary, for example, after one week or after two weeks.

TABLE S1
Itolizumab Sequences

| SEQ | | |
|---|---|---|
| ID | | |
| NO: | Description | Sequence |
| 1 | Light chain | Asp Ile Gln Met Thr Gln Ser Pro Ser Ser Leu Ser |
| | variable | Ala Ser Val Gly Asp Arg Val Thr Ile Thr Cys Lys |
| | region | Ala Ser Arg Asp Ile Arg Ser Tyr Leu Thr Trp Tyr |
| | | Gln Gln Lys Pro Gly Lys Ala Pro Lys Thr Leu Ile |
| | | Tyr Tyr Ala Thr Ser Leu Ala Asp Gly Val Pro Ser |
| | | Arg Phe Ser Gly Ser Gly Ser Gly Gln Asp Tyr Ser |
| | | Leu Thr Ile Ser Ser Leu Glu Ser Asp Asp Thr Ala |
| | | Thr Tyr Tyr Cys Leu Gln His Gly Glu Ser Pro Phe |
| | | Thr Leu Gly Ser Gly Thr Lys Leu Glu Ile Lys |
| 2 | Heavy chain | Glu Val Gln Leu Val Glu Ser Gly Gly Gly Leu Val |
| | variable | Lys Pro Gly Gly Ser Leu Lys Leu Ser Cys Ala Ala |
| | region | Ser Gly Phe Lys Phe Ser Arg Tyr Ala Met Ser Trp |
| | | Val Arg Gln Ala Pro Gly Lys Arg Leu Glu Trp Val |
| | | Ala Thr Ile Ser Ser Gly Gly Ser Tyr Ile Tyr Tyr |
| | | Pro Asp Ser Val Lys Gly Arg Phe Thr Ile Ser Arg |
| | | Asp Asn Val Lys Asn Thr Leu Tyr Leu Gln Met Ser |
| | | Ser Leu Arg Ser Glu Asp Thr Ala Met Tyr Tyr Cys |
| | | Ala Arg Arg Asp Tyr Asp Leu Asp Tyr Phe Asp Ser |
| | | Trp Gly Gln Gly Thr Leu Val Thr Val Ser Ser |
| 3 | Heavy chain | gaagtgcagc tggtggagtc tgggggaggc ttagtgaagc |
| | variable | ctggagggtc cctgaaactc tcctgtgcag cctctggatt |
| | nucleotide | caagtttagt agatatgcca tgtcttgggt tcgccaggct |
| | sequence | ccggggaaga ggctggagtg ggtcgcaacc attagtagtg |
| | | gtggtagtta catctactat ccagacagtg tgaagggtcg |
| | | attcaccatc tccagagaca atgtcaagaa caccctgtat |
| | | ctgcaaatga gcagtctgag gtctgaggac acggccatgt |
| | | attactgtgc aagacgagat tacgacctgg actactttga |
| | | ctcctggggc caaggcaccc ttgtcaccgt ctcctca |
| 4 | Light chain | gacatccaga tgacccagtc tccatcctcc ctgtctgcat |
| | variable | cggtgggaga cagagtcact atcacttgca aggcgagtcg |
| | nucleotide | ggacattaga agctatttaa cctggtacca gcagaaacca |
| | sequence | gggaaagctc ctaagaccct gatctattat gcaacaagct |
| | | tggcagatgg ggtcccgtcg agattcagtg gcagtggatc |
| | | tgggcaagat tattctctca ccatcagcag cctggagtct |
| | | gacgatacag caacttacta ctgtctacaa catggtgaga |
| | | gtccattcac gctcggctcg gggaccaagc tggaaatcaa a |
| 5 | Heavy chain | Glu Val Gln Leu Val Glu Ser Gly Gly Gly Leu Val |
| | full amino | Lys Pro Gly Gly Ser Leu Lys Leu Ser Cys Ala Ala |
| | acid | Ser Gly Phe Lys Phe Ser Arg Tyr Ala Met Ser Trp |
| | sequence | Val Arg Gln Ala Pro Gly Lys Arg Leu Glu Trp Val |
| | | Ala Thr Ile Ser Ser Gly Gly Ser Tyr Ile Tyr Tyr |
| | | Pro Asp Ser Val Lys Gly Arg Phe Thr Ile Ser Arg |
| | | Asp Asn Val Lys Asn Thr Leu Tyr Leu Gln Met Ser |
| | | Ser Leu Arg Ser Glu Asp Thr Ala Met Tyr Tyr Cys |
| | | Ala Arg Arg Asp Tyr Asp Leu Asp Tyr Phe Asp Ser |
| | | Trp Gly Gln Gly Thr Leu Val Thr Val Ser Ser Ala |
| | | Ser Thr Lys Gly Pro Ser Val Phe Pro Leu Ala Pro |
| | | Ser Ser Lys Ser Thr Ser Gly Gly Thr Ala Ala Leu |
| | | Gly Cys Leu Val Lys Asp Tyr Phe Pro Glu Pro Val |
| | | Thr Val Ser Trp Asn Ser Gly Ala Leu Thr Ser Gly |
| | | Val His Thr Phe Pro Ala Val Leu Gln Ser Ser Gly |
| | | Leu Tyr Ser Leu Ser Ser Val Val Thr Val Pro Ser |
| | | Ser Ser Leu Gly Thr Gln Thr Tyr Ile Cys Asn Val |
| | | Asn His Lys Pro Ser Asn Thr Lys Val Asp Lys Lys |
| | | Val Glu Pro Lys Ser Cys Asp Lys Thr His Thr Cys |
| | | Pro Pro Cys Pro Ala Pro Glu Leu Leu Gly Gly Pro |
| | | Ser Val Phe Leu Phe Pro Pro Lys Pro Lys Asp Thr |
| | | Leu Met Ile Ser Arg Thr Pro Glu Val Thr Cys Val |
| | | Val Val Asp Val Ser His Glu Asp Pro Glu Val Lys |
| | | Phe Asn Trp Tyr Val Asp Gly Val Glu Val His Asn |
| | | Ala Lys Thr Lys Pro Arg Glu Glu Gln Tyr Asn Ser |
| | | Thr Tyr Arg Val Val Ser Val Leu Thr Val Leu His |
| | | Gln Asp Trp Leu Asn Gly Lys Glu Tyr Lys Cys Lys |
| | | Val Ser Asn Lys Ala Leu Pro Ala Pro Ile Glu Lys |
| | | Thr Ile Ser Lys Ala Lys Gly Gln Pro Arg Glu Pro |
| | | Gln Val Tyr Thr Leu Pro Pro Ser Arg Asp Glu Leu |
| | | Thr Lys Asn Gln Val Ser Leu Thr Cys Leu Val Lys |
| | | Gly Phe Tyr Pro Ser Asp Ile Ala Val Glu Trp Glu |
| | | Ser Asn Gly Gln Pro Glu Asn Asn Tyr Lys Thr Thr |
| | | Pro Pro Val Leu Asp Ser Asp Gly Ser Phe Phe Leu |
| | | Tyr Ser Lys Leu Thr Val Asp Lys Ser Arg Trp Gln |
| | | Gln Gly Asn Val Phe Ser Cys Ser Val Met His Glu |
| | | Ala Leu His Asn His Tyr Thr Gln Lys Ser Leu Ser |
| | | Leu Ser Pro Gly Lys |
| 6 | Light chain | Asp Ile Gln Met Thr Gln Ser Pro Ser Ser Leu Ser |
| | full amino | Ala Ser Val Gly Asp Arg Val Thr Ile Thr Cys Lys |
| | acid | Ala Ser Arg Asp Ile Arg Ser Tyr Leu Thr Trp Tyr |
| | sequence | Gln Gln Lys Pro Gly Lys Ala Pro Lys Thr Leu Ile |
| | | Tyr Tyr Ala Thr Ser Leu Ala Asp Gly Val Pro Ser |
| | | Arg Phe Ser Gly Ser Gly Ser Gly Gln Asp Tyr Ser |
| | | Leu Thr Ile Ser Ser Leu Glu Ser Asp Asp Thr Ala |
| | | Thr Tyr Tyr Cys Leu Gln His Gly Glu Ser Pro Phe |
| | | Thr Leu Gly Ser Gly Thr Lys Leu Glu Ile Lys Arg |
| | | Thr Val Ala Ala Pro Ser Val Phe Ile Phe Pro Pro |
| | | Ser Asp Glu Gln Leu Lys Ser GlyThr Ala Ser Val Val |
| | | Cys Leu Leu Asn Asn Phe Tyr Pro Arg Glu Ala Lys |
| | | Val Gln Trp Lys Val Asp Asn Ala Leu Gln Ser Gly |
| | | Asn Ser Gln Glu Ser Val Thr Glu Gln Asp Ser Lys |
| | | Asp Ser Thr Tyr Ser Leu Ser Ser Thr Leu Thr Leu |
| | | Ser Lys Ala Asp Tyr Glu Lys His Lys Val Tyr Ala |
| | | Cys Glu Val Thr His Gln Gly Leu Ser Ser Pro Val |
| | | Thr Lys Ser |
| | | Phe Asn Arg Gly Glu Cys |
| 7 | VHCDR1 | GFKFSRYAMS |
| 8 | VHCDR2 | TISSGGSYIYYPDSVKG |
| 9 | VHCDR3 | RDYDLDYFDS |
| 10 | VLCDR1 | KASRDIRSYLT |
| 11 | VLCDR2 | YATSLAD |
| 12 | VLCDR3 | LQHGESP |

The section headings used herein are for organizational purposes only and are not to be construed as limiting the subject matter described.

The Examples which follow are set forth to aid in understanding the disclosure but are not intended to and should not be construed to limit its scope in any way. The Examples do not include detailed descriptions for conventional methods employed in the assay procedures. Such methods are well known to those of ordinary skill in the art and are described in numerous publications including by way of examples.

### EXAMPLES

These examples are provided for illustrative purposes only and not to limit the scope of the claims provided herein.

### Example 1

### In Vitro Loss of Surface Levels of CD6 was Analyzed Using Flow Cytometry on Cryopreserved PBMCs

Cryopreserved peripheral blood mononuclear cells (PBMCs) were thawed and resuspended in complete media (RPMI+10% FBS+1× penicillin/streptomycin). PBMCs were counted and the cell concentration was adjusted to 4 million cells per mL in complete media. Itolizumab and isotype treatment was prepared in complete media at 2× the final treatment concentration. PBMCs were treated with either itolizumab or isotype in a 24W TC-treated plate by combining 250 uL of PBMCs and 250 uL of 2× treatment into a single well for a final volume of 500 uL. Treated PBMCs were incubated at 37° C. for specific timepoints (10, 30, 60, 120, 180, 1440 minutes). At each timepoint, all cells were harvested from designated wells and transferred to FACs wash buffer at 4° C. (FWB, 1×PBS+2% FBS+0.5 g NaN₃). Cells were pelleted at 1500 rpm for 5 minutes and supernatant was removed. Viability staining and Fc blocking occurred at room temperature. Surface levels of CD6 was detected using a proprietary PE-conjugated anti-CD6 antibody clone that does not compete with itolizumab binding. The results are shown in FIG. 1. Experiments were also performed on a cell line (see FIG. 14).

### Example 2

### Surface Levels of CD6 from Patients Following Treatment with Itolizumab was Analyzed Using Flow Cytometry on Fresh Whole Blood

Whole blood (WB) was collected from patients at specific timepoints per the clinical protocol and shipped overnight for analysis the next day. Red blood cells (RBCs) were lysed at a 9:1 ratio (1× lyse buffer to WB). Following RBC lysis, cells were resuspended in staining buffer and transferred to staining plate for detection of surface levels of CD6. Cells were stained with a proprietary non-competing anti-CD6 antibody clone and fluorescently labeled with PE to measure the total amount of CD6 receptors on the cell surface.

### Example 3

### PBMCs from Normal Healthy Volunteers were Immunophenotyped to Identify Changes in Immune Cell Populations Following Treatment with Itolizumab

Whole blood was collected from normal healthy volunteers following treatment with itolizumab and PBMCs were isolated from WB by density gradient centrifugation, and cryopreserved in a standard manner. For analysis, cryopreserved PBMCs were thawed, washed and fluorescently labeled with antibodies targeting CD3, CD4, CD6, CD8, CD25, CCR6, HELIOS, and FOXP3 to identify changes in immune cell subsets such as naïve T cells, T_{eff} and T_{reg} cells by flow cytometry.

### Example 4

### Ratio of T_{reg} Cells to CD4+ Cells was Determined by Measuring Cell Type-Specific Epigenetic Markers in DNA Using Epiontis ID

Epiontis ID enables accurate counting of cell types by measuring cell type-specific epigenetic markers using qPCR. Cryopreserved PBMCs from patients treated with itolizumab, which contain a mixture of target and nontarget cells were put through a bisulfite sequence conversion of specific demethylated DNA sequences which are only demethylated in target cells. DNA was purified, specially designed PCR primers which only amplify bisulfite-converted targets were added and qPCR was performed. The following epigenetic qPCR assays were used to analyze patient samples (assay/cell type): FOXP3 (T_{reg} cells), CD4 (CD4 T cells), CD8B (CD8 T cells), PD1 positive cells, LRP5 (B cells), LCN2 (Neutrophils), MVD (NK cells), S1PR1 positive cells, PRG2 (Eosinophils), CBX6 (Memory B cells), CCR7 positive cells, S1PR5 positive cells, IL17A (Th17 cells).

### Example 5

### Binding Characteristics of Itolizumab to Low, Medium, and High Densities of CD6 was Analyzed Using Surface Plasmon Resonance (SPR)

Analysis was conducted at 25° C. in an HBS-P buffer system (10 mM HEPES pH 7.4, 150 mM NaCl, and 0.05% Surfactant P20) using a Biacore 3000 optical biosensor equipped with a streptavidin (SA) sensor chip. Biotinylated recombinant human CD6 was captured to Fc2, Fc3, and Fc4 of the sensor chip to a low (7 RU), medium (55 RU), and high (255 RU) density, respectively. These densities are similar to low, medium, and high levels of CD6 observed on T cells.

| Chip Surface | Rhu CD6 (RU) | Estimated CD6 binding sites/μm² |
|---|---|---|
| Low | 7 | 120 |
| Medium | 55 | 783 |
| High | 255 | 3493 |

Analyte concentration series ranging from 900 nM or 0.137 nM were prepared using 3-fold dilutions in running buffer. The association phases for all analyte concentration were monitored for 240 s, at a flow rate of 50 uL/min while the dissociation phases were collected for 1800 s, at a flow rate of 50 uL/min. The surface was regenerated with 1 M MgCl2 for 15 s, at a flow rate of 50 uL/min. The results are shown in FIGS. 12A-12F and FIGS. 13A-13F.

### Example 6

### Itolizumab Induces Cleavage of Cell Surface CD6

PBMCs were treated with isotype or itolizumab for 72 hours at 37 C. After 72h, cells and supernatant were collected.

Cell associated CD6. CD6 level on the cell surface was analyzed by flow cytometry. The cell pellet was used to isolate total proteins using RIPA buffer with 0.1% triton. Soluble CD6 was Immunoprecipitated from the supernatant using a specific anti-CD6 antibody that recognize the extracellular portion of CD6. Soluble CD6 levels in the supernatant were also analyzed by MSD.

CD6 in cell supernatant. CD6 levels were further analyzed in the Immunoprecipitated supernatant and in the cell lysate by western blot. Proteins were loaded on a 4-12% Nu-Page gel and transferred on a PVDF membrane. The membrane was stained with the Ponceau and then incubated overnight at 4 C with a primary Antibody anti-CD6 or anti-gapdh. The day after, the membrane was washed with TBS+tween and incubated 1h at RT with an HRP-conjugated secondary antibody, washed again with TBS+tween and the signal was acquired, after adding the HRP substrate, using a chemiluminescence imager. The results are shown in FIGS. 18A-18B, and FIG. 19.

### Example 7

### The Role of Monocytes in Itolizumab-Induced Cleavage of CD6

To assess the contribution of other cell types to itolizumab-induced cleavage of CD6, T cells, monocytes, NK cells, and B cells were isolated from PBMCs by negative magnetic separation. Isolated T cells were then treated with isotype or itolizumab in the presence of monocytes, NK cells, or B cells. Isolated T cells were also treated with isotype or itolizumab in the presence of increasing ratios of T cells to monocytes.

Following treatment, surface levels of CD6 was detected on the surface of T cells by flow cytometry using an anti-CD6 detection antibody that does not interfere with itolizumab binding. The results are shown in FIGS. 20A-20B. FIG. 20A shows that loss of CD6 on T cells is only observed when monocytes, and to a lesser extent, NK cells, are present during treatment. FIG. 20B shows that an increased loss of CD6 is observed with increasing numbers of monocytes.

### Example 8

### The Effects of Itolizumab on T Cell Characteristics and Function

Ninety-six well flat bottom plates were coated with CD3+ALCAM (in PBS) for 2 hrs at 37° C. After the 2 hr incubation, plates were washed once with 1×PBS followed by a blocking step with 5% BSA for 30 mins at 37° C. After blocking, plates were washed with 1×PBS twice.

Frozen PBMCs were thawed gently in complete RPMI. Cells were adjusted at 2×10^{\hat{ }6} cells/ml. Cells were then seeded at 200 k cells/well in 100 uls. Itolizumab was prepared at a 3-fold dilution curve in complete media at a 3×. Final assay volume is 200 uls in a 96 flat bottom plate.

PBMCs were incubated for a total of 24 hrs at 37 C. Plates were spun down in which the supernatant was collected to detect cytokines. Cells were then collected to detect loss of CD6 on CD4 and CD8 T cells and a decrease in activation markers. A non-competing CD6 antibody was used in order to assess the loss of CD6 by flow cytometry. CD6 was reported as geometric mean fluorescence (gMFI). The results are shown in FIGS. 21A-21E, which evidence that decreased cell surface levels of CD6 correlate with decreased T cell activation markers CD71, PD-1, CD25, IL-2, and TNFa.

PBMCs were treated with isotype control or itolizumab for 24 hours to generate CD6^{high} and CD6^{low} cells, respectively. Following the 24-hour treatment, cells were collected and washed repeatedly with media to remove residual antibody treatment. Washed cells were counted and the concentration was adjusted to 1×10^{\hat{ }6} cells/mL.

96 well flat-bottom plates were coated with CD3+ALCAM in PBS for 2 hours at 37° C. After the 2-hour incubation, plates were washed twice with PBS. Washed CD6 high and CD6^{low} cells were plated at 200 k cells/well and incubated for 24, 48, 72, and 96 hours. At each designated time point, T cell activation was assessed by surface and intracellular staining for transcription factors using flow cytometry. The results are shown in FIG. 22, which evidences that CD6^{low} cells generated by initial incubation with itolizumab not only remained CD6^{low} but were also less T_{eff}-like (e.g., less Tₕ1 and Tₕ17-like) following subsequent stimulation with CD3+ALCAM. In contrast, CD6^{high} cells generated by incubation with isotype control were more T_{eff}-like in their characteristics following subsequent stimulation.

### Example 9

### Itolizumab Reduces Alloreactivity of T Lymphocytes

A one-way mixed lymphocyte reaction was used to assess the utility of itolizumab in reducing alloreactivity of T cells. PBMC responder and stimulator pairings were identified, and responder counts at time of takedown were used as a readout. Responder PBMCs were thawed, labeled with cell trace violet, and pretreated with isotype or itolizumab for 2 hours. Stimulator PBMCs were thawed and treated with Mitomycin C for 20 minutes to inhibit proliferation. Following Mitomycin C treatment, stimulator PBMCs were repeatedly washed with media. Responder and stimulator PBMCs were counted and mixed at a 1:1 ratio in a 96 well U-bottom with itolizumab or isotype treatment.

Responder cells were stimulated in the presence of stimulator cells after approximately 96-168 hours. At each time point, cells were collected, and proliferation and activation of responder cells was assessed by flow cytometry. The results are shown in FIGS. 23A-23D. Here, responder cells treated with itolizumab expressed lower levels of CD6 (23A), were less proliferative as shown by lower CD4+ counts (23B) and levels of CD71 after 168 hours (23D), and were less activated as shown by lower levels of CD25 after 168 hours (23C).

### Example 10

### Itolizumab is Useful in Enriching for T_{reg} Lymphocytes

The ability of itolizumab to enrich for CD6^{low} T cells was used as a basis for testing the utility of generating T_{reg} lymphocytes from itolizumab-enriched CD6^{low} T cells, relative to generating the T_{reg} lymphocytes from CD6^{high} T cells.

Generation of CD6^{low} and CD6^{high} T cells. Frozen PBMCs were thawed gently in complete RPMI. Total PBMCs were treated with isotype control or itolizumab for 24 hrs at 37 C. After 24 hrs cells were collected for confirmation of loss of CD6 on CD4 T cells and to isolate naïve T cells.

Differentiation of T_{regs} from naïve T cells. Those PBMCs that were treated as mentioned above were enriched for naïve T cells with a STEMCELL kit. To differentiate T_{regs} from the CD6^{low} and CD6^{high}, CD3/CD28 activator was added in combination with a T_{reg} differentiation cocktail from STEMCELL for a total of 7 days.

Confirmation of T_{reg} phenotype. After 7 days under T_{reg} differentiation conditions, CD6^{low} and CD6^{high} were collected for intracellular staining and acquired by a flow cytometer. Cells were confirmed by Foxp3 and Helios co-expression. The results in FIGS. 24A-24B evidence that pre-treatment of PBMCs with itolizumab enriches for CD6^{low} naïve T cells, which can be used to more efficiently generate higher numbers of T_{regs} relative to pre-treatment with isotype control.

T_{reg} Suppression Assay. At day 7 of differentiation, CD6^{low} and CD6^{high} Tregs were collected. Tregs were counted and seeded at 100 k, 50 k, and 25 k to generate three ratios 1:1, 1:2, and 1:4.

Generation of T responder cells. Frozen PBMCs from the same donor were gently thawed. PBMCs were enriched for CD4 T cells that were CD25−, a total of two kits were combined to generate these cells. Both kits were from STEMCELL. Once these cells were isolated, cells were labeled with Cell Trace Violet.

Stimulation of T responders and co-culture with T_{regs}. T responders were added to the wells with Tregs. Conditions tested were unstimulated and varying levels of stimuli. Suppression assay was cultured for a total of 4 days at 37° C.

Calculating suppression of T_{responders}. Tresponders that were cultured alone with varying levels of stimuli were measured for their proliferation of cell trace violet. Conventional gating method calculated suppression of the Tresponders proliferation. The results are shown in FIG. 25. Here, T_{regs} generated from CD6^{low} naïve T cells have increased suppressive activity relative to CD6^{high} T_{regs} at all T_{reg} to responder T cell ratios; the greatest suppression is observed at a 1:1, T_{reg}:T_{responder} ratio.

These observations evidence that loss of surface CD6 (as induced by itolizumab) skews the differentiation of naïve T cells into Tregs that have a greater capacity to suppress overactive & pathogenic immune cell function which contributes to restoring balance to the immune system.

To summarize, pre-treatment of naïve T cells with itolizumab leads to a T_{reg} phenotype that has increased Foxp3 and HELIOS co-expression, and this increase in frequency and MFI has been shown to correlate with greater suppression activity.

### Example 11

### Prevention of GVHD in a Patient at Risk Using Selective Enrichment of CD6′/Depletion of CD6^{high} Cells From the Tissue to be Engrafted

It will be apparent to one skilled in the art how to use known methods in combination with the methods of the present disclosure to decrease the risk of GVHD in a patient by using an optimized anti-CD6 antibody to strip CD6 from CD6^{high} cells in a population of umbilical cord blood cells or bone marrow cells or HLA-matching sibling cells prior to transplantation. Cells may be expanded ex vivo before or after treatment with an optimized anti-CD6 antibody to decrease cell surface expression of CD6 to prevent GVHD in the transplant recipient.

For instance, itolizumab can be used to treat human cord blood samples obtained from normal full-term deliveries. The cord blood units may be red cell depleted and may undergo clinical grade selection of CD34+ cells prior to treatment with the anti-CD6 antibody, antigen-binding fragment, or fusion anti-CD6 antibody.

The CD6 stripped samples can be administered to a patient in need of stem cell therapy and at risk for GVHD. One or more biomarkers will be measured and the patient will be closely monitored for positive and negative clinical response.

Progenitor cells pretreated with anti-CD6 antibody, antigen-binding fragment thereof, or fusion antibody can be used to prevent GVHD in a patient in need of stem cell transplantation. Prior to transplantation the umbilical cord blood transplant or bone marrow transplant or HLA-matching transplant is treated with itolizumab in an amount sufficient to reduce the quantity of CD6 transplanted by 10%, 20%, 30%, 40%, 50%, 60%, 70%, 80%, or more than 80%. CD6 levels can be monitored using conventional techniques known in the art, such as by FACS analysis of cells or serum analysis from a blood sample withdrawn from the patient. For instance, a physician of skill in the art can withdraw a blood sample from the patient at various time points and determine the extent of cell surface CD6 by conducting a FACS analysis. A physician of skill in the art can evaluate the clinical manifestations of GVHD after administering to the human patient an antibody, antigen-binding fragment thereof, ADC, or soluble ligand capable of binding CD6, such as an anti-CD6 antibody described herein.

### Example 12

### Itolizumab Potency Assay

A potency assay to assess the functionality of itolizumab by loss of CD6 has been developed. PBMCs were seeded at 200 k cells/well in 100 uls. Itolizumab was prepared at a 3-fold dilution curve in complete media at a 3×. Final assay volume is 200 uls in a 96 flat bottom plate.

PBMCs were incubated for a total of 24 hrs at 37 C. Plates were spun down in which the supernatant was collected to detect soluble CD6. Cells were then collected to detect loss of CD6 on CD4 and CD8 T cells. A non-competing CD6 antibody was used in order to assess the loss of CD6 by flow cytometry. CD6 was reported as geometric mean fluorescence (gMFI). The results are shown in FIGS. 26A-26B. The results here evidence that the assay is robust and reproducible, shows excellent sensitivity to changes in protein concentration (see 26A), and is sensitive enough to detect changes within the Fc region of the antibody (see 26B). Changes in the mAb that affect binding to CD6 or the Fc-receptor would lead to reduced loss of CD6, and thus serve as a good measure for the potency of itolizumab batches.

In summary, Itolizumab induces cleavage of CD6 in a dose-dependent and time-dependent manner, and the degree of CD6 loss correlates with reductions in T cell activation and cytokine expression. Loss of CD6 is observed in patients dosed with itolizumab, and itolizumab induces cleavage in a robust manner across multiple donors. As shown herein, cleavage of CD6 is thus a surrogate for the effect of the drug, and in vitro assays to measure cleavage of CD6 provide a robust and reproducible approach that is relevant to the clinic.

### Example 13

### Lupus (SLE) Patients Treated with Itolizumab have Decreased CD6 Expression on CD4 and CD8 T Cells

Surface levels of CD6 on CD4 and CD8 T cells from SLE subjects (from the EQUALISE trial) following itolizumab treatment were analyzed and as expressed by average fluorescence of CD6. While baseline (pre-drug) levels of CD6 is variable across subjects, loss of surface CD6 was observed across all doses (see FIGS. 27A-27E). Surface levels of CD6 throughout the course of the study is more variable on subjects treated with a lower dose of itolizumab (0.4 mg/kg). Subjects treated with a higher dose of itolizumab (0.8-3.2 mg/kg) maintain low levels of CD6 weeks after the last dose. Arrows indicate when subjects received itolizumab dose. Dashed line indicates no surface CD6 as determined by the fluorescence minus one (FMO) control. Statistics shown for CD6 on CD4 T cells compared to baseline. Total N=26 subjects included in analysis across the five dose cohorts. FIGS. 28A-28B show that cell surface CD6 on CD4 (28A) and CD8 (28B) T cells decreases following the first dose of itolizumab, and that a greater loss of surface CD6 is observed with higher doses of itolizumab. Data is expressed as % of baseline (pre-drug) and shown for Day 15 (14 days post the first dose, pre the second). **p<0.01, *p<0.05.

### Example 14

### Itolizumab Modulates the Tₕ17:T_{reg} Ratio in Patients

PBMCs sampled from subjects in the EQUIP study (4 subjects dosed at 0.8 mg/kg and 1 placebo subject) were immunophenotyped using epiontisID. As shown in FIGS. 29A-29B, treatment with itolizumab at 0.8 mg/kg caused a 3-fold reduction in Tₕ17 cells and a 2.5-fold increase in T_{reg} cells as compared to placebo.

While certain embodiments of the present disclosure have been shown and described herein, it will be apparent to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

1. A method for determining an optimal dosage of itolizumab in a human subject having an autoimmune, immuno-inflammatory, or inflammatory disease, graft versus host disease (GVHD), or organ transplant rejection, comprising:
(a) determining a baseline of cell surface CD6 levels in a tissue sample from the subject, wherein the tissue sample comprises T-lymphocytes, and optionally defining a target level of cell surface CD6 in the subject;
(b) administering to the subject a series of two or three or more dosages of itolizumab, optionally increasing dosages;
(c) monitoring cell surface CD6 levels in a tissue sample from the subject between the series of dosages, wherein the tissue sample comprises T-lymphocytes;
(d) identifying the lowest dosage from the series of dosages as being the optimal dosage if cell surface CD6 levels in step (c) are about or less than about 5, 10, 15, 20, 25, 30, 40, 45, or 50 percent of the baseline from (a), or are within about or less than about 5, 10, 15, or 20 percent of the optional target level from (a), and if no further reductions in cell surface CD6 levels are observed between the series of dosages.

2. The method of claim 1, comprising determining cell surface CD6 levels on CD4 cells and/or CD8 cells in the tissue sample from the subject.

3. The method of claim 1 or 2, wherein the tissue sample is a blood sample.

4. The method of any one of claims 1-3, comprising defining the target level of cell surface CD6 in the subject based on clinical parameters or symptoms of the disease.

5. A dosing regimen for treatment of an autoimmune, immuno-inflammatory, or inflammatory disease, graft versus host disease (GVHD), or organ transplant rejection in a human subject in need thereof, comprising:
(a) determining a baseline of cell surface CD6 levels in a tissue sample from the subject, wherein the tissue sample comprises T-lymphocytes, and optionally defining a target level of cell surface CD6 in the subject;
(b) administering to the subject a dosage of itolizumab, which reduces cell surface CD6 levels in T-lymphocytes in the subject to about or less than about 5, 10, 15, 20, 25 percent of the baseline from (a);
(c) monitoring cell surface CD6 levels in a tissue sample from the subject, wherein the tissue sample comprises T-lymphocytes; and
(d) administering a further dosage of itolizumab to the subject before or if the cell surface CD6 levels in (c) return to about or more than about 25, 30, 35, 40, 45, 50, 55, 60, 65, 70, or 75 percent of the baseline from (a), or rise to about or above the target level from (a).

6. The dosing regimen of claim 5, which maintains cell surface CD6 levels in T-lymphocytes (optionally CD4 and/or CD8 cells) from the subject at about or lower than about 40, 45, 50, 55, 60, 65, 70, or 75 percent of the baseline from (a), or within about 5, 10, 15, or 20 percent of the optional target level from (a), optionally defining the target level of cell surface CD6 in the subject based on clinical parameters or symptoms of the disease.

7. A dosing regimen for treatment of an autoimmune, immuno-inflammatory, or inflammatory disease, or graft versus host disease (GVHD) or organ transplant rejection in a human subject in need thereof, comprising:
(a) determining a baseline level of CD6^{high} T-lymphocytes in a blood sample from the subject, and optionally defining a target level of CD6^{high} T-lymphocytes;
(b) administering to the subject a dosage of itolizumab, which reduces the level of CD6^{high} T-lymphocytes in the subject to about or less than about 5, 10, 15, 20, 25, 30, or 40 percent of the baseline from (a);
(c) monitoring levels of CD6^{high} T-lymphocytes in a blood sample from the subject; and
(d) administering a further dosage of itolizumab to the subject before the levels of CD6^{high} T-lymphocytes from (c) return to about or more than about 25, 30, 35, 40, 45, 50, 55, 60, 65, 70, or 75 percent of the baseline level from (a), or rise to about or above the target level from (a), optionally defining the target level of CD6^{high} T-lymphocytes in the subject based on clinical parameters or symptoms of the disease.

8. The dosing regimen of claim 5, which maintains levels of CD6^{high} T-lymphocytes in the subject at about or less than about 45, 50, 55, 60, 65, 70, or 75 percent of the baseline level from (a), optionally wherein the T-lymphocytes are CD4 cells and/or CD8 cells.

9. The dosing regimen of any one of claims 5-8, which decreases the ratio of CD6^{high}:CD6^{low} T-lymphocytes in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally wherein the T-lymphocytes are CD4 cells and/or CD8 cells.

10. The dosing regimen of any one of claims 5-8, which decreases the ratio of T_{eff}:T_{reg} cells in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally CD4 cells and/or CD8 cells, optionally wherein the T_{eff} cells are Th17 cells.

11. A method of preventing or ameliorating graft versus host disease (GVHD) in a human transplant patient comprising:
(a) incubating a transplant tissue with an anti-CD6 antibody, optionally itolizumab, for a time sufficient to reduce cell surface levels of CD6 on CD6^{high} T-lymphocytes in the transplant tissue; and
(b) transplanting the transplant tissue into the patient.

12. The method of claim 11, comprising determining cell surface levels of CD6 on T-lymphocytes in the transplant tissue before and after step (a), and performing step (b) if cell surface levels of CD6 after step (a) are reduced by about or at least about 10, 20, 30, 40, 50, 60, 70, 80, 90, 100, 200, 300, 400, 500 percent or more relative to before step (a).

13. The method of claim 11 or 12, wherein the transplant tissue comprises umbilical cord blood cells, bone marrow cells, peripheral blood cells, mobilized peripheral blood cells, mesenchymal stem cells, hematopoietic stem cells, cells differentiated from stem cells/progenitor cells, engineered cells (optionally chimeric antigen receptor (CAR) cells), or any combinations of said cells.

14. The method of any one of claims 11-13, wherein the transplant tissue is autologous to the human transplant patient.

15. The method of any one of claims 11-13, wherein the transplant tissue is allogeneic to the human transplant patient.

16. A method for treatment or amelioration of an autoimmune, immuno-inflammatory, or inflammatory disease in a human patient in need thereof, comprising:
(a) incubating a transplant tissue with an anti-CD6 antibody, optionally itolizumab, for a time sufficient to reduce cell surface levels of CD6 on CD6^{high} T-lymphocytes in the transplant tissue, thereby generating transplant tissue enriched for CD6^{low} T-lymphocytes;
(b) treating the transplant tissue from (a) for a time sufficient to generate T_{reg} lymphocytes from the CD6^{low} T-lymphocytes; and
(c) transplanting the transplant tissue from (c) into the patient.

17. The method of claim 16, comprising determining cell surface levels of CD6 on T-lymphocytes in the transplant tissue before and after step (a), and performing step (b) if cell surface levels of CD6 after step (a) are reduced by about or at least about 10, 20, 30, 40, 50, 60, 70, 80, 90, 100, 200, 300, 400, 500 percent or more relative to before step (a).

18. The method of claim 16 or 17, wherein (b) comprises incubating the transplant tissue enriched for CD6^{low} T-lymphocytes with a combination of cytokines, growth factors, and transcription factors for a time sufficient to generate T_{reg} lymphocytes.

19. The method of any one of claims 16-18, wherein the transplant tissue comprises umbilical cord blood cells, bone marrow cells, peripheral blood cells, mobilized peripheral blood cells, mesenchymal stem cells, hematopoietic stem cells, cells differentiated from stem cells/progenitor cells, engineered cells (optionally chimeric antigen receptor (CAR) cells), or any combinations of said cells

20. The method of any one of claims 16-19, wherein the transplant tissue is autologous to the human patient.

21. The method of any one of claims 16-19, wherein the transplant tissue is allogeneic to the human patient.

22. A method for treatment of an autoimmune, immuno-inflammatory, or inflammatory disease, graft versus host disease (GVHD), or organ transplant rejection in a human subject in need thereof, comprising:
(a) administering itolizumab to the subject; and
(b) determining levels of cell surface CD6 on T-lymphocytes in a tissue sample from the subject, determining levels of CD6^{high} and optionally CD6^{low} T-lymphocytes in a tissue sample from the subject, and/or determining levels of T_{eff} and T_{reg} cells in the subject, wherein the tissue sample comprises T-lymphocytes;
wherein the administration of itolizumab reduces any one or more of (i) levels of cell surface CD6 on T-lymphocytes, optionally CD4 and/or CD8 cells; (ii) levels of CD6^{high} T-lymphocytes in the subject; (iii) the ratio of CD6^{high}:CD6^{low} T-lymphocytes in the subject; and/or (iv) the ratio of T_{eff}:T_{reg} cells in the subject, and thereby reduces a pathogenic immune response in the subject.

23. The method of claim 22, wherein the administration of itolizumab:
decreases cell surface CD6 levels on T-lymphocytes in the subject by about or at least about 10, 20, 30, 40, 50, 60, 70, 80, 90, 100, 200, 300, 400, 500 percent or more relative to a control or standard or baseline, optionally wherein the T-lymphocytes are CD4 cells and/or CD8 cells;
decreases levels of CD6^{high} T-lymphocytes in the subject by about or at least about 10, 20, 30, 40, 50, 60, 70, 80, 90, 100, 200, 300, 400, 500 percent or more relative to a control or standard or baseline, optionally wherein the T-lymphocytes are CD4 cells and/or CD8 cells;
decreases the ratio of CD6^{high}:CD6^{low} T-lymphocytes in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally wherein the T-lymphocytes are CD4 cells and/or CD8 cells; and/or
decreases the ratio of T_{eff}:T_{reg} cells in the subject by about or at least about 10, 20, 30, 40, 50, 60, 70, 80, 90, 100, 200, 300, 400, 500 percent or more or by about or at least about 1.5, 2, 3, 4, 5, 6, 7, 8, 9, 10-fold or more relative to a control or standard or baseline, optionally wherein the T_{eff} cells are Th17 cells.

24. An in vitro cell-based method for analyzing a test lot of itolizumab, comprising
(a) incubating the test lot of itolizumab with cells that express CD6 on the cell surface;
(b) measuring cell surface CD6 expression on the cells; and
(c) formulating the test lot of itolizumab as a pharmaceutical composition if the test lot decreases cell surface CD6 expression relative to a control or standard, and rejecting the test lot of itolizumab if the test lot does not decrease cell surface CD6 expression relative to the control or standard.

25. The method of claim 24, wherein (b) comprises directly measuring cell surface CD6 expression by flow cytometry, cytometry by time-of-flight (CyToF), cellular ELISA, or immunofluorescent microscopy, or wherein (b) comprises measuring soluble CD6 in supernatant as an indicator of cell surface CD6 expression, optionally by enzyme-linked immunosorbent assay (ELISA), chemiluminescence assay, electrochemiluminescence assay, high performance liquid chromatography, western blot, or immunoprecipitation followed by western blot.

26. The method of claim 24 or 25, wherein the cells comprise peripheral blood mononuclear cell (PBMCs).

27. The method of any one of claims 24-26, wherein the cells comprise a human T cell line or a cell line engineered to express CD6, optionally human CD6.

28. The method of claim 27, wherein the cell line is selected from MOLT-4, MOLT-3, MOLT-16, HuT 78, HuT 102, Jurkat, Jurkat NFAT, CCRF-CEM, 12.1, MJ (G11), LOUCY, SUP-T1, HEL.92.1.7, EFO-21, RPMI-8226, HPB-ALL, HH, KE37, P12ICHIKAWA, PEER, ALLSIL, RPMI8402, CMLT1, PF382, EHEB, and DU4475 cells.

29. The method of any one of claims 24-28, wherein the cells comprise monocytes, optionally a monocyte cell line.

30. The method of claim 29, wherein the monocyte cell line is selected from U937, THP1, MC-1010, TUR, AML-193, and MV-4-11.

31. The method of any one of claims 27-30, wherein (a) the human T cell line or cell line engineered to express CD6 and (b) the monocytes, optionally monocyte cell line, are present at a ratio of about 30:1, 25:1, 20:1, 15:1, 10:1, 5:1, 4:1, 3:1, 2:1, 1:1, 1:2, 1:3, 1:4, 1:5, or 1:10.

32. The method of any one of claims 24-31, comprising formulating the test lot of itolizumab as a pharmaceutical composition if it decreases cell surface CD6 expression by about or at least about 10, 20, 30, 40, 50, 60, 70, 80, 90, 100, 200, 300, 400, 500 percent or more relative to the control or standard and/or if it increases soluble CD6 in supernatant by about or at least about 10, 20, 30, 40, 50, 60, 70, 80, 90, 100, 200, 300, 400, 500 percent or more relative to the control or standard.

33. A method of screening an anti-CD6 antibody, or antigen binding fragment thereof, for use as a biological therapeutic comprising:
(a) incubating the candidate anti-CD6 antibody, or antigen binding fragment thereof, with cells that express CD6 on the cell surface;
(b) measuring cell surface CD6 expression on the cells; and
(c) formulating the candidate anti-CD6 antibody, or antigen binding fragment thereof, as a pharmaceutical composition if it decreases cell surface CD6 expression relative to a control or standard.

34. The method of claim 21, wherein (b) comprises directly measuring cell surface CD6 expression by flow cytometry, cytometry by time-of-flight (CyToF), cellular ELISA, or immunofluorescent microscopy, or wherein (b) comprises measuring soluble CD6 in supernatant as an indicator of cell surface CD6 expression, optionally by enzyme-linked immunosorbent assay (ELISA), chemiluminescence assay, electrochemiluminescence assay, high performance liquid chromatography, western blot, and immunoprecipitation followed by western blot.

35. The method of claim 33 or 34, wherein the cells comprise peripheral blood mononuclear cell (PBMCs).

36. The method of any one of claims 33-35, wherein the cells comprise a human T cell line or a cell line engineered to express CD6, optionally human CD6.

37. The method of claim 36, wherein the cell line is selected from MOLT-4, MOLT-3, MOLT-16, HuT 78, HuT 102, Jurkat, Jurkat NFAT, CCRF-CEM, 12.1, MJ (G11), LOUCY, SUP-T1, HEL.92.1.7, EFO-21, RPMI-8226, HPB-ALL, HH, KE37, P12ICHIKAWA, PEER, ALLSIL, RPMI8402, CMLT1, PF382, EHEB, and DU4475 cells.

38. The method of any one of claims 33-37, wherein the cells comprise monocytes, optionally a monocyte cell line.

39. The method of claim 38, wherein the monocyte cell line is selected from U937, THP1, MC-1010, TUR, AML-193, and MV-4-11.

40. The method of any one of claims 33-39, wherein (a) the human T cell line or cell line engineered to express CD6 and (b) the monocytes, optionally monocyte cell line, are present at a ratio of about 30:1, 25:1, 20:1, 15:1, 10:1, 5:1, 4:1, 3:1, 2:1, 1:1, 1:2, 1:3, 1:4, 1:5, or 1:10.

41. The method of any one of claims 33-40, comprising formulating the candidate anti-CD6 antibody, or antigen binding fragment thereof, as a pharmaceutical composition if it decreases cell surface CD6 expression by about or at least about 10, 20, 30, 40, 50, 60, 70, 80, 90, 100, 200, 300, 400, 500 percent or more relative to a control or standard and/or if it increases soluble CD6 in supernatant by about or at least about 10, 20, 30, 40, 50, 60, 70, 80, 90, 100, 200, 300, 400, 500 percent or more relative to the control or standard.

42. The method or dosing regimen of any one of claims 1-23, wherein the subject or patient with the autoimmune, immuno-inflammatory, or inflammatory disease has an increased ratio of T_{eff}:T_{reg} cells relative to a standard or healthy subject, optionally wherein the T_{eff} cells are Th17 cells.

43. The method or dosing regimen of claim 42, wherein the ratio of T_{eff}:T_{reg} cells is increased by about or at least about 1.5, 2, 3, 4, 5, 6, 7, 8, 9, or 10-fold or more relative to the standard or healthy subject.

44. The method or dosing regimen of any one of claim 1-23 or 42-43, wherein the autoimmune, immuno-inflammatory, or inflammatory disease is inflammatory bowel disease (IBD), optionally Crohn's disease or ulcerative colitis, systemic lupus erythematosus (SLE), optionally SLE with lupus nephritis, rheumatoid arthritis (RA), multiple sclerosis (MS), psoriasis, psoriatic arthritis, ankyolosing spondylitis, or asthma.
